(19) United States
(12) Patent Application Publication
(10) Pub. No.: US 2025/0235113 A1
(43) Pub. Date: Jul. 24, 2025

(54) DEVICE AND METHOD FOR MEDICAL DIAGNOSTICS

(71) Applicant: Poltorak Technologies, LLC, Pomona, NY (US)

(72) Inventor: Alexander Poltorak, Monsey, NY (US)

(21) Appl. No.: 19/031,942

(22) Filed: Jan. 18, 2025

Related U.S. Application Data
(63) Continuation of application No. 16/995,749, filed on Aug. 17, 2020, now Pat. No. 12,251,201.
(60) Provisional application No. 62/888,213, filed on Aug. 16, 2019.

(51) Int. Cl. A61B 5/0205; A61B 5/00; A61B 5/021; A61B 5/024; A61B 5/029; A61B 5/0533; A61B 5/08; A61B 5/091; A61B 5/369; A61B 5/389; G16H 50/30; G16H 50/50; G16H 50/70

(52) U.S. Cl. CPC A61B 5/02055; A61B 5/0022; A61B 5/029; A61B 5/0533; A61B 5/369; A61B 5/389; A61B 5/6802; A61B 5/6847; A61B 5/7264; A61B 5/7275; A61B 5/746; G16H 50/30; A61B 5/021; A61B 5/024; A61B 5/02416; A61B 5/0816; A61B 5/091; A61B 5/7239; A61B 2503/40; A61B 2560/0257; A61B 2562/0247; A61B 2562/0257; A61B 2562/0271; G16H 50/50; G16H 50/70

(57) ABSTRACT

A method and device for analysis of sampled physiological parameters according to at least a second-order process over time, e.g., second-order differential equation or model, for use in therapeutic, diagnostic, or predictive health applications. The system may generate an alert regarding a present or predicted health abnormality. The biometric device may be implantable, wearable, contact or non-contact, and may communicate through a network, to send an alert. The system may further comprise an actuator or therapeutic device to perform an action based on the at least second-order process.

### CROSS REFERENCE TO RELATED APPLICATIONS

The present application is a Continuation of U.S. patent application Ser. No. 16/995,749, filed Aug. 17, 2020, now pending, which is a Non-provisional of, and claims benefit of priority under 35 U.S.C. § 119(e), U.S. Provisional Patent Application No. 62/888,213, filed Aug. 16, 2019, the entirety of which are expressly incorporated herein by reference.

### FIELD OF THE INVENTION

The present invention pertains to medical diagnostic devices, medical implants, and wearable devices and, more particularly, to a device and method for measurement of physiological parameters to assess changes in health or medical conditions.

### BACKGROUND OF THE INVENTION

All patents, references, and other information cited herein are expressly incorporated herein by reference in their entirety.

Biological organisms are homeostatically controlled, which means that feedback or other control mechanisms are present which, tend to maintain constant (quasistatic) internal conditions over a range of external conditions. These biological systems may be complex and interrelated, but often, at their basic level, there is a response to the perturbation that returns to nominal. Physical disease or pathology may relate to a change in the nominal conditions, or a defect in the mechanisms that return the system to normal. These features imply that a second or higher-order differential equation or equivalent construct in various representational domains can model the biological system, and that a large set of such equations or constructs can model an organism. Modern medicine evolved from a time prior to automation, and mainstream practitioners and their regulatory agencies mainly rely on values and thresholds (zeroth order) for determining health. Rarely are rates (first-order) employed, except in some specialties, such as nephrology and pharmacology, when considering metabolic and excretion rates.

Thus, in modern medicine, a patient's physiological parameters may represent actionable signals. For example, blood oxygen saturation below 90% may indicate a need for immediate attention, such as oxygen supplementation. Similarly, trends may be actionable, such as increasing prostate-specific antigen (PSA). However, more complex temporal analysis is typically not performed due to lack of normalization, data sampling irregularities, etc. Perhaps more importantly, there is typically no accepted underlying theory for action based on complex temporal analysis, and rather typical actions are predicated on a level or rate only, and not higher-order considerations.

Biometrics is the technical term for body measurements and calculations. It refers to metrics related to physiological characteristics. The biometric system can be used for various purposes ranging from measurement of a physiological parameter to the assessment of any medical condition.

The proliferation of medical implants and, particularly, wearable devices, present a rich source of biometric data available for collection, analysis, and action dependent thereon.

Generally, most data come as a mixture of patterns and random noise. It is usually the objective of data analysis to separate patterns from noise. However, the data discarded as “noise” is not necessarily meaningless, and its omission from the analysis is often more as a matter of convenience than correctness. The classification of data as “noise” often depends on the choice of the filter or the knowledge of the underlying dynamics (the model). In the absence of the model, often quite meaningful data is discarded labeled as “noise.”

In the general case of analog processes described by smooth differentiable functions, the pattern may be modeled as a differential equation, whereas the noise is modeled by the constants arising out of the integration of the differential equation required for this solution. In other cases, noise may be modeled as a Gaussian or other statistical process.

Let us consider, for example, the law of motion, described by the second-order differential equation, also known as the Second Law of Newton: m×d²x/dt²=F, where m is the mass of an object, F is the force acting on the object, and d²x/dt² is the second derivative of the coordinate x over time t. Alternatively, this equation can be expressed as m\ddot{x}=F, where two dots over x signify the second derivative over time: \ddot{x}=d²x/dt², or as ma=F, where a is the acceleration, a=d²x/dt². To solve this differential equation of the second-order, we need to integrate it twice. As it is well known, integration, which is the reverse of differentiation, involves supplying constants (because a derivative of a constant is always zero). Integrating the equation twice, therefore, produces two constants not defined by the differential equation itself, that need to be constrained by choosing the initial conditions for both. Where the system being modeled is known, these initial conditions may be real and ascertainable values.

Integrating the equation of motion the first time, gives the velocity (the first derivative of the position over time) plus or minus an arbitrary constant, which is constrained by choosing the initial velocity. Integrating the equation of motion the second time, gives us the position x plus or minus an arbitrary constant, which is constrained by choosing the initial position. During differentiation, these constants disappear, revealing the pattern: ma=F.

In Analytical Mechanics (see en.wikipedia.org/wiki/Analytical_mechanics), the equation of motion emerges from Euler-Lagrange or Hamiltonian formalism, which ultimately links the law of motion with energy conservation. Lagrangian mechanics (see en.wikipedia.org/wiki/Lagrangian_mechanics), Hamiltonian mechanics (see en.wikipedia.org/wiki/Hamiltonian_mechanics), Principle of Least Action (see en.wikipedia.org/wiki/Principle_of_least_action) are all different ways of describing evolution in time of a dynamical system in a deterministic way.

Hooke's law is a law of physics that states that the force (F) needed to extend or compress a spring by some distance x scales linearly with respect to that distance: Fₛ=kx, where k is a constant characterizing the spring's stiffness, and x is the displacement, which is small compared to the total possible deformation of the spring.

Similarly, in biology, all live organisms, cells, closed systems, and subsystems strive to maintain homeostasis. In this regard, they act as “springs” in a sense that, when displaced from the equilibrium under stress, they strive to return to equilibrium. The “elasticity” or “stiffness” of the biological system is accomplished by self-correcting mechanisms that engage in restoring the homeostasis under stress. The loss of the ability to effectively maintain homeostasis and return to equilibrium state under stress may be a sign of a disease or a result of aging. The evolution in time of physiological parameters could be modeled similarly to mechanical systems using tools of analytical mechanics. The disease process may be reflected in shortening or lengthening of the timescale, roughly corresponding to a natural frequency, and energy efficiency and overshoot, roughly corresponding to damping.

When measuring blood chemistry, an analyte sensor may be employed. The analyte sensor may be an implantable glucose sensor, such as described with reference to U.S. Pat. No. 6,001,067 and U.S. Patent Publication No. 2005/0027463; a transcutaneous glucose sensor, such as described with reference to U.S. Patent Publication No. 2006/0020187; the sensor may be configured to be implanted in a host vessel or extracorporeally, such as is described in U.S. Patent Publication No. 2007/0027385, U.S. Patent Publication No. 2008/0119703, U.S. Patent Publication No. 2008/0108942, and U.S. Patent Publication No. 2007/0197890; the continuous glucose sensor comprises a transcutaneous sensor such as described in U.S. Pat. No. 6,565,509, for example; a subcutaneous sensor such as described with reference to U.S. Pat. No. 6,579,690 or U.S. Pat. No. 6,484,046; a refillable subcutaneous sensor such as described with reference to U.S. Pat. No. 6,512,939; an intravascular sensor such as described with reference to U.S. Pat. No. 6,477,395; or an intravascular sensor such as described with reference to U.S. Pat. No. 6,424,847.

Examples of systems and methods for processing sensor analyte data are described in more detail herein and in U.S. Pat. Nos. 7,310,544 and 6,931,327 and U.S. Patent Publication Nos. 2005/0043598, 2007/0032706, 2007/0016381, 2008/0033254, 2005/0203360, 2005/0154271, 2005/0192557, 2006/0222566, 2007/0203966 and 2007/0208245, all of which are incorporated herein by reference in their entirety for all purposes.

Biocompatible implants are well-known. See, U.S. Pat. Nos. 5,764,518; 6,379,669; 6,567,259; 7,097,662; 7,333,013; 7,371,825; 7,414,534; 7,713,923; 7,727,143; 7,765,005; 7,780,590; 7,787,958; 7,813,778; 7,842,092; 7,876,228; 7,916,013; 7,932,825; 7,956,162; 7,966,075; 7,981,025; 7,983,435; 7,983,763; 8,000,801; 8,036,736; 8,078,282; 8,079,518; 8,081,925; 8,093,991; 8,114,345; 8,114,964; 8,192,406; 8,197,454; 8,200,342; 8,202,260; 8,207,316; 8,246,533; 8,251,946; 8,257,729; 8,269,635; 8,269,636; 8,301,243; 8,321,032; 8,323,232; 8,326,435; 8,348,882; 8,374,697; 8,389,286; 8,444,653; 8,454,552; 8,457,757; 8,457,760; 8,478,378; 8,483,840; 8,486,070; 8,496,657; 8,509,913; 8,543,199; 8,557,772; 8,574,146; 8,577,453; 8,577,465; 8,577,468; 8,577,478; 8,623,023; 8,639,524; 8,644,957; 8,666,471; 8,673,194; 8,707,040; 8,715,159; 8,718,776; 8,721,520; 8,721,643; 8,751,013; 8,784,425; 8,788,057; 8,790,400; 8,795,260; 8,795,359; 8,798,773; 8,805,478; 8,805,530; 8,808,163; 8,808,373; 8,814,868; 8,838,249; 8,849,368; 8,855,785; 8,875,714; 8,901,084; 8,911,486; 8,926,573; 8,929,999; 8,946,390; 8,975,372; 8,989,867; 8,989,870; 9,002,471; 9,011,361; 9,017,380; 9,026,792; 9,031,637; 9,044,209; 9,044,612; 9,055,791; 9,061,139; 9,061,151; 9,067,073; 9,072,560; 9,113,844; 9,125,981; 9,126,825; 9,144,488; 9,144,489; 9,149,189; 9,159,223; 9,161,693; 9,187,539; 9,198,911; 9,204,798; 9,211,185; 9,237,012; 9,248,291; 9,248,302; 9,251,960; 9,271,857; 9,288,614; 9,308,381; 9,317,920; 9,326,720; 9,326,730; 9,327,061; 9,333,071; 9,339,188; 9,339,372; 9,345,404; 9,356,473; 9,357,922; 9,358,378; 9,358,392; 9,361,572; 9,367,793; 9,370,618; 9,370,619; 9,386,360; 9,392,939; 9,398,854; 9,403,009; 9,403,021; 9,409,018; 9,414,651; 9,414,775; 9,415,163; 9,420,856; 9,420,857; 9,421,388; 9,424,508; 9,427,053; 9,427,160; 9,427,189; 9,427,190; 9,436,903; 9,445,651; 9,445,730; 9,462,856; 9,462,962; 9,463,012; 9,474,461; 9,474,888; 9,486,168; 9,492,656; 9,492,678; 9,498,195; 9,501,735; 9,514,338; 9,517,023; 9,522,282; 9,526,422; 9,526,650; 9,530,089; 9,532,716; 9,532,738; 9,539,037; 9,542,685; 9,545,506; 9,553,486; 9,564,777; 9,569,719; 9,569,720; 9,576,236; 9,579,422; 9,579,510; 9,582,748; 9,582,749; 9,585,722; 9,603,997; 9,610,391; 9,634,921; 9,636,509; 9,655,558; 9,662,015; 9,672,393; 9,675,273; 9,675,809; 9,693,777; 9,700,234; 9,704,209; 9,723,898; 9,724,098; 9,724,183; 9,731,104; 9,732,322; 9,757,124; 9,804,672; 9,826,963; 9,833,353; 9,839,422; 9,839,423; 9,854,370; 9,874,923; 9,876,537; 9,878,159; 9,884,150; 9,884,456; 9,895,301; 9,901,276; 9,918,716; 9,919,099; 9,936,890; 9,943,697; 9,950,166; 9,974,705; 9,986,924; 10,028,659; 10,028,743; 10,034,743; 10,039,661; 10,045,764; 10,045,798; 10,064,624; 10,066,203; 10,070,992; 10,105,081; 10,117,621; 10,124,182; 10,130,476; 10,164,685; 10,176,412; 10,179,065; 10,182,819; 10,186,760; 10,188,394; 10,194,802; 10,196,596; 10,220,217; 20020001588; 20030053284; 20050181973; 20050197677; 20050247319; 20060047283; 20060212096; 20070027371; 20070120683; 20070154030; 20070179562; 20070219639; 20070265704; 20070282196; 20080004642; 20080020012; 20080048855; 20080049376; 20080065181; 20080097496; 20080102096; 20080106419; 20080180242; 20080207983; 20080208010; 20080234598; 20080288027; 20080303728; 20090012372; 20090024161; 20090028957; 20090062825; 20090069869; 20090099626; 20090118683; 20090148496; 20090155900; 20090157147; 20090157151; 20090163980; 20090163981; 20090198293; 20090202387; 20090206087; 20090227862; 20090254179; 20090274737; 20090281597; 20090305972; 20100015201; 20100063347; 20100082102; 20100094654; 20100139672; 20100143871; 20100144641; 20100145337; 20100152573; 20100160997; 20100161004; 20100168821; 20100174240; 20100174349; 20100191236; 20100191306; 20100217239; 20100217240; 20100217241; 20100217242; 20100217243; 20100217244; 20100222686; 20100222802; 20100261526; 20100274121; 20100311640; 20100312081; 20100317955; 20100318160; 20100321163; 20100324578; 20100324579; 20100324639; 20100331868; 20100331874; 20100331932; 20110022140; 20110023343; 20110029043; 20110040343; 20110043297; 20110057037; 20110063088; 20110066079; 20110074349; 20110098576; 20110124983; 20110130636; 20110152756; 20110237861; 20110249381; 20110251516; 20110264058; 20110275930; 20110288600; 20110305672; 20110319785; 20120008714; 20120053585; 20120058106; 20120059434; 20120123221; 20120161901; 20120190386; 20120203079; 20120223705; 20120226118; 20120232012; 20120234433; 20120277859; 20120296399; 20120302874; 20130023954; 20130030255; 20130035544; 20130053711; 20130070387; 20130078244; 20130085408; 20130092564; 20130116664; 20130116665; 20130116666; 20130116667; 20130131679; 20130195806; 20130198463; 20130233324; 20130238056; 20130243799; 20130253297; 20130253660; 20130268029; 20130289529; 20130317584; 20130338494; 20130338768; 20130338769; 20130338770; 20130338771; 20130338772; 20130338773; 20130345561; 20140012111; 20140018644; 20140045757; 20140062717; 20140065153; 20140073704; 20140073839; 20140081076; 20140163644; 20140221732; 20140236105; 20140239528; 20140245783; 20140245784; 20140245785; 20140245786; 20140245787; 20140245788; 20140245789; 20140245790; 20140245791; 20140246497; 20140246498; 20140246499; 20140246500; 20140246501; 20140246502; 20140246917; 20140247136; 20140247137; 20140247142; 20140247143; 20140247144; 20140247146; 20140247147; 20140247149; 20140247150; 20140247151; 20140247154; 20140247155; 20140247156; 20140249379; 20140249760; 20140249853; 20140273824; 20140277277; 20140285396; 20140288619; 20140288647; 20140296663; 20140296978; 20140328517; 20140330244; 20140330256; 20140330257; 20140330347; 20140330357; 20140343691; 20140358196; 20140358197; 20140371821; 20140371824; 20140376336; 20140379090; 20150011860; 20150025478; 20150057595; 20150066124; 20150071934; 20150073498; 20150073499; 20150073500; 20150077050; 20150080982; 20150080992; 20150088226; 20150094547; 20150099959; 20150100108; 20150100109; 20150116053; 20150129664; 20150174296; 20150179038; 20150183828; 20150194052; 20150196378; 20150196409; 20150221208; 20150231402; 20150238277; 20150289911; 20150327989; 20150360038; 20150365738; 20150366915; 20150367144; 20150374541; 20160023007; 20160030650; 20160030756; 20160038324; 20160051825; 20160058324; 20160186140; 20160191120; 20160220198; 20160228034; 20160228052; 20160235317; 20160235318; 20160274752; 20160278638; 20160287380; 20160303313; 20160310048; 20160310737; 20160317095; 20160317797; 20160325083; 20160325084; 20160331518; 20160335632; 20160342882; 20160358063; 20160358155; 20160358156; 20160359222; 20160361009; 20160374556; 20170007420; 20170020241; 20170020402; 20170028185; 20170056677; 20170071510; 20170072121; 20170100214; 20170106196; 20170117739; 20170127929; 20170152486; 20170173216; 20170197072; 20170209666; 20170209705; 20170216610; 20170216611; 20170228627; 20170230084; 20170231738; 20170232256; 20170258585; 20170259072; 20170270721; 20170272316; 20170281927; 20170281928; 20170281957; 20170296834; 20170304635; 20170312530; 20170316487; 20170333080; 20170340872; 20170348146; 20170368158; 20180001018; 20180008185; 20180021235; 20180021498; 20180021510; 20180028275; 20180028827; 20180036053; 20180036115; 20180050189; 20180050214; 20180055500; 20180059126; 20180060520; 20180085038; 20180085592; 20180103879; 20180126053; 20180126133; 20180138022; 20180147413; 20180154075; 20180168811; 20180184944; 20180188704; 20180188714; 20180188715; 20180192952; 20180200003; 20180200185; 20180210425; 20180210426; 20180210427; 20180214690; 20180214694; 20180221663; 20180233016; 20180243567; 20180243573; 20180243577; 20180253073; 20180253074; 20180253075; 20180255374; 20180255375; 20180255376; 20180255377; 20180255378; 20180255379; 20180255380; 20180255381; 20180255382; 20180255383; 20180280694; 20180284735; 20180284736; 20180284737; 20180284741; 20180284742; 20180284743; 20180284744; 20180284745; 20180284746; 20180284747; 20180284749; 20180284752; 20180284753; 20180284754; 20180284755; 20180284756; 20180284757; 20180284758; 20180299878; 20180310824; 20180310964; 20180321666; 20180321667; 20180321672; 20180322445; 20180353219; 20180360355; 20180372720; 20190000656; 20190025805; 20190025806; 20190025812; 20190025813; 20190033845; 20190033846; 20190033847; 20190033848; 20190033849; 20190038214; 20190038496; 20190041835; 20190041836; 20190041840; 20190041841; 20190041842; 20190041843; 20190041844; 20190041845; 20190041846; 20190053712; 20190053915; 20190054284; 20190064791; 20190064792; 20190070350; 20190072922; 20190072923; 20190072924; 20190072925; 20190072926; 20190072928;

Medical devices may communicate directly with a server or caregiver device, or indirectly through a relay or mesh network. Swarm methods may be used for the relay of messages, selection of supernodes for forwarding of messages in an ad hoc network, distributed processing of tasks, etc. Security and privacy are preferably maintained through cryptographic communications and hardware security.

The implant may have functions of other types of RF-ID tags, such as access, control, feeding and consumption management, etc. For example, a pet door, feeder, etc., may receive identification signals from the implant, and selectively authorize or react to the identification.

Various distributed ledger and blockchain technologies may be employed for data permanence, transparency, authentication, and in some cases, transfer of economic value or authority.

See, U.S. Pat. Nos. 9,014,661; 9,351,124; 9,436,923; 9,569,771; 9,641,342; 9,818,092; 9,820,120; 9,849,364; 9,853,819; 9,855,785; 9,862,222; 9,922,380; 9,922,381; 9,942,304; 10,005,564; 10,022,613; 10,022,614; 10,026,118; 10,039,113; 10,046,228; 10,055,715; 10,078,839; 10,080,498; 10,103,936; 10,108,938; 10,115,068; 10,120,888; 10,121,186; 10,127,247; 10,129,032; 10,135,835; 10,142,312; 10,147,076; 10,152,756; 10,160,251; 10,163,079; 10,163,080; 10,164,952; 10,168,693; 10,172,409; 10,176,418; 10,176,481; 10,178,105; 10,178,890; 10,192,198; 10,193,695; 10,195,513; 10,200,199; 10,200,834; 10,204,160; 10,225,085; 20140368601; 20150269624; 20150356524; 20150356555; 20160012465; 20160098723; 20160098730; 20160170996; 20160192166; 20160203522; 20160203572; 20160224803; 20160300252; 20160321654; 20160379312; 20170017936; 20170017954; 20170017955; 20170028622; 20170031874; 20170033932; 20170046652; 20170046689; 20170046694; 20170046799; 20170046806; 20170048209; 20170048234; 20170048235; 20170083907; 20170085545; 20170085555; 20170091756; 20170109735; 20170132615; 20170132630; 20170140408; 20170161517; 20170173262; 20170206532; 20170221032; 20170221052; 20170228706; 20170228731; 20170228734; 20170232300; 20170236177; 20170236196; 20170237569; 20170237570; 20170243177; 20170243208; 20170243209; 20170243212; 20170243213; 20170243214; 20170243217; 20170243222; 20170243286; 20170243287; 20170244707; 20170244721; 20170250796; 20170256000; 20170256001; 20170256003; 20170262862; 20170300905; 20170300910; 20170300946; 20170316487; 20170345105; 20170358041; 20170364860; 20170373849; 20180001184; 20180006990; 20180012311; 20180013815; 20180019984; 20180039512; 20180041345; 20180069899; 20180071789; 20180072415; 20180072416; 20180074481; 20180074488; 20180074521; 20180074522; 20180074523; 20180075386; 20180075406; 20180078843; 20180081787; 20180081955; 20180082043; 20180082295; 20180083786; 20180089627; 20180089641; 20180089669; 20180094953; 20180096121; 20180096175; 20180108024; 20180115600; 20180117446; 20180117447; 20180120225; 20180123804; 20180130034; 20180130050; 20180130158; 20180131765; 20180133583; 20180136633; 20180137461; 20180137506; 20180137512; 20180139057; 20180144298; 20180144342; 20180150816; 20180158036; 20180165738; 20180167394; 20180173906; 20180174097; 20180174188; 20180176017; 20180181806; 20180181909; 20180181964; 20180182140; 20180183796; 20180189528; 20180189854; 20180191503; 20180198617; 20180198876; 20180203755; 20180204034; 20180204111; 20180211213; 20180211718; 20180218003; 20180218354; 20180225649; 20180227354; 20180232693; 20180232730; 20180232817; 20180253430; 20180253805; 20180257306; 20180259976; 20180261307; 20180262493; 20180264347; 20180268360; 20180268418; 20180268479; 20180268483; 20180284093; 20180285709; 20180293577; 20180300772; 20180307854; 20180307859; 20180307959; 20180314868; 20180315141; 20180322164; 20180324407; 20180326291; 20180330369; 20180336515; 20180349893; 20180357603; 20180357725; 20180365633; 20180369437; 20180369438; 20180374037; 20180375750; 20180376336; 20190005507; 20190005566; 20190007381; 20190008117; 20190012637; 20190019144; 20190019171; 20190026690; 20190034536; 20190034605; 20190034808; 20190034888; 20190034889; 20190034923; 20190035499; 20190036887; 20190038791; 20190043008; 20190043010; 20190044736; 20190046863; 20190049931; 20190050888; 20190053470; 20190056726; 20190057454; 20190065733; 20190066063;

There are two typical ways to approach a systems biology issue. First involves generating a descriptive or physical model of the system, i.e., a model where the components and their parameters have physical correlates, and therefore assuming the complexity of the model is sufficient, the model may be populated with measured parameters, and insights from the model will reflect on the identifiable components of the system. A second approach employs a statistical optimization process to produce an output trained to correspond to a set of inputs and corresponding actions, e.g., an artificial neural network (ANN) or deep neural network (DNN). Neural network technologies rarely implement an optimization, which represents an architecture correlated to a physical system, unless the attributes of the physical system are design constraints, and so the internal (hidden) states of the network do not correspond to real parameters and are not indicative of actual states or events. Neural networks may be trained (i.e., statistically optimized) to respond to time-domain changes, and may directly estimate derivatives, e.g., second derivatives, from time-series data.
Bishop, Chris M. “Training with noise is equivalent to Tikhonov regularization.” Neural computation 7, no. 1 (1995): 108-116.
Bishop, Chris. “Exact calculation of the Hessian matrix for the multilayer perceptron.” (1992): 494-501.
Buntine, Wray L., and Andreas S. Weigend. “Computing second derivatives in feed-forward networks: A review.” IEEE transactions on Neural Networks 5, no. 3 (1994): 480-488.
Hirasawa, Kotaro, Masanao Ohbayashi, and Jun-ichi Murata. “Universal learning network and computation of its higher order derivatives.” In Proceedings of ICNN'95-International Conference on Neural Networks, vol. 3, pp. 1273-1277. IEEE, 1995.
Hirasawa, Kotaro, Masanao Ohbayashi, Masaru Koga, and Masaaki Harada. “Forward propagation universal learning network.” In Proceedings of International Conference on Neural Networks (ICNN'96), vol. 1, pp. 353-358. IEEE, 1996.
LeCun, Yann, Ido Kanter, and Sara A. Solla. “Second order properties of error surfaces: Learning time and generalization.” In Advances in neural information processing systems, pp. 918-924. 1991.
Opitz, David W. “Analyzing the structure of a neural network using principal component analysis.” In Proceedings of International Conference on Neural Networks (ICNN'97), vol. 1, pp. 254-259. IEEE, 1997.
Pearlmutter, Barak A. “Fast exact multiplication by the Hessian.” Neural computation 6, no. 1 (1994): 147-160.
Piché, Stephen W. “The second derivative of a recurrent network.” In Proceedings of 1994 IEEE International Conference on Neural Networks (ICNN'94), vol. 1, pp. 245-250. IEEE, 1994.
Popa, Calin-Adrian. “Exact Hessian Matrix Calculation for Complex-Valued Neural Networks.” In International Workshop Soft Computing Applications, pp. 439-455. Springer, Cham, 2014.
Williams, Peter M. “Bayesian regularization and pruning using a Laplace prior.” Neural computation 7, no. 1 (1995): 117-143.

Therefore, while internal states of a neural network implementation typically do not correspond to physical elements emulated by the neural network, the exposed inputs, and outputs of a neural network may be trained or constrained to correspond to physical correlates, including the dynamic coefficients of a higher order differential equation or response profile. Thus, instead of purely analytic methods for defining the higher-order (e.g., order >1) derivatives, a neural network or other statistical optimization technique may be employed to extract the parameters.

### Homeostasis

The characteristic of biological systems is that physiological parameters are subject to homeostasis, and thus, after a perturbation, tend to return to a nominal value. According to control theory, this return to nominal value after a perturbation requires a greater than first-degree response, i.e., a higher-order response. A first-order response can, in theory, only attenuate the perturbation, but not correct it. Thus, homeostatic mechanisms are typically higher order, or within the biological system act at a higher order.

In biology, homeostasis is the state of steady internal physical and chemical conditions maintained by living systems. This dynamic state of equilibrium is typically considered the condition of optimal functioning for the organism and includes many variables, such as body temperature and fluid balance, being kept within certain pre-set limits (homeostatic range). (Note that the basis for the optimization is complex, and therefore may not correspond to simple survival, sped, size, etc.). Other variables include the pH of extracellular fluid, the concentrations of sodium, potassium, and calcium ions, as well as that of the blood sugar level, and these need to be regulated despite changes in the environment, diet, or level of activity. Each of these variables is controlled by one or more regulators or homeostatic mechanisms, which together maintain life. See, en.wikipedia.org/wiki/Homeostasis.

Homeostasis is brought about by a natural resistance or response to change in the conditions from an “optimal” state, and equilibrium is typically maintained by many regulatory mechanisms. Homeostatic control mechanisms tend to have, according to one theory, at least three interdependent components for the variable being regulated: a receptor, a control center, and an effector. The receptor is the sensing component that monitors and responds to changes in the environment, either external or internal. Receptors include thermoreceptors and mechanoreceptors. Control centers include, for example, the respiratory center, and the renin-angiotensin system. An effector is a target acted on, to bring about the change back to the normal state. At the cellular level, receptors include nuclear receptors that bring about changes in gene expression through up-regulation or down-regulation and act in negative feedback mechanisms. An example of this is in the control of bile acids in the liver.

Some centers, such as the renin-angiotensin system, control more than one variable. When the receptor senses a stimulus, it reacts by sending action potentials to a control center. The control center sets the maintenance range—the acceptable upper and lower limits—for the particular variable, such as temperature. The control center responds to the signal by determining an appropriate response and sending signals to an effector, which can be one or more muscles, an organ, or a gland. When the signal is received and acted on, negative feedback is provided to the receptor that stops the need for further signaling.

The metabolic processes of all organisms can only take place in very specific physical and chemical environments. The conditions vary with each organism, and with whether the chemical processes take place inside the cell or in the interstitial fluid bathing the cells. The best-known homeostatic mechanisms in humans and other mammals are regulators that keep the composition of the extracellular fluid (or the “internal environment”) constant, especially with regard to the temperature, pH, osmolality, and the concentrations of sodium, potassium, glucose, carbon dioxide, and oxygen. However, a great many other homeostatic mechanisms, encompassing many aspects of human physiology, control other entities in the body. Where the levels of variables are higher or lower than those needed, they are often prefixed with hyper- and hypo-, respectively, such as hyperthermia and hypothermia and hypertension and hypotension.

If a system is homeostatically controlled, it does not imply that its value is necessarily absolutely steady, i.e., that it maintains a constant value or steady-state in health. Core body temperature is, for instance, regulated by a homeostatic mechanism with temperature receptors in, amongst others, the hypothalamus of the brain. However, the set point of the regulatory pathway is regularly reset. For instance, core body temperature in humans varies during the course of the day (i.e., has a circadian rhythm), with the lowest temperatures occurring at night, and the highest in the afternoons. Other normal temperature variations include those related to the menstrual cycle. The temperature regulator's set point is reset during infections to produce a fever. Organisms are capable of adjusting somewhat to varied conditions such as temperature changes or oxygen levels at altitude, by process of acclimatization.

Homeostasis does not govern every activity in the body. For instance, the signal (be it via neurons or hormones) from the receptor or sensor to the control center is, of necessity, highly variable in order to convey information about the direction and magnitude of the detected error. Similarly, the effector's response needs to be highly adjustable to reverse the error; in fact, it should be very nearly in proportion (but in the opposite direction) to the error that is threatening the internal environment. For instance, the arterial blood pressure in mammals is homeostatically controlled and measured by stretch receptors in the walls of the aortic arch and carotid sinuses at the beginnings of the internal carotid arteries. The sensors send messages via sensory nerves to the medulla oblongata of the brain, indicating whether the blood pressure has fallen or risen, and by how much. The medulla oblongata then distributes messages via the motor or efferent nerves belonging to the autonomic nervous system to a wide variety of effector organs, whose activity is consequently changed to reverse the error in the blood pressure. Thus, where the physiological measurement is of a parameter that itself comprises a feedback signal to another core body system, the parameter may itself not be homeostatically controlled, and would not be expected to achieve a consistent value over time.

One of the effector organs for homeostasis is the heart, whose rate is stimulated to rise (tachycardia) when the arterial blood pressure falls, or to slow down (bradycardia) when the pressure rises above nominal value. Thus, the heart rate (for which there is no sensor in the body) is not homeostatically controlled but is one of the effector responses to errors in the arterial blood pressure and other parameters. Another example is the rate of sweating. This is one of the effectors in the homeostatic control of body temperature, and therefore highly variable in rough proportion to the heat load that threatens to destabilize the body's core temperature, for which there is a sensor in the hypothalamus of the brain.

Mammals regulate their core temperature using input from thermoreceptors in the hypothalamus, brain, spinal cord, internal organs, and great veins. Apart from the internal regulation of temperature, a process called allostasis can come into play that adjusts behavior to adapt to the challenge of very hot or cold extremes (and to other challenges). These adjustments may include seeking shade and reducing activity, or seeking warmer conditions and increasing activity, or huddling. When core temperature falls, the blood supply to the skin is reduced by intense vasoconstriction. The blood flow to the limbs (which have a large surface area) is similarly reduced and returned to the trunk via the deep veins that lie alongside the arteries (forming venae comitantes). This acts as a counter-current exchange system that short-circuits the warmth from the arterial blood directly into the venous blood returning into the trunk, causing minimal heat loss from the extremities in cold weather. The subcutaneous limb veins are tightly constricted, not only reducing heat loss from this source but also forcing the venous blood into the counter-current system in the depths of the limbs. The metabolic rate is increased, initially by non-shivering thermogenesis, followed by shivering thermogenesis if the earlier reactions are insufficient to correct the hypothermia.

When core temperature rises are detected by thermoreceptors, the sweat glands in the skin are stimulated via cholinergic sympathetic nerves to secrete sweat onto the skin, which, when it evaporates, cools the skin and the blood flowing through it. Panting is an alternative effector in many vertebrates, which cools the body also by the evaporation of water, but this time from the mucous membranes of the throat and mouth.

Blood sugar levels are regulated within fairly narrow limits. In mammals, the primary sensors for this are the beta cells of the pancreatic islets. The beta cells respond to a rise in the blood sugar level by secreting insulin into the blood and simultaneously inhibiting their neighboring alpha cells from secreting glucagon into the blood. This combination (high blood insulin levels, and low glucagon levels) act on effector tissues, chief of which are the liver, fat cells, and muscle cells. The liver is inhibited from producing glucose, taking it up instead, and converting it to glycogen and triglycerides. The glycogen is stored in the liver, but the triglycerides are secreted into the blood as very-low-density lipoprotein (VLDL) particles that are taken up by adipose tissue, there to be stored as fats. The fat cells take up glucose through special glucose transporters (GLUT4), whose numbers in the cell wall are increased as a direct effect of insulin acting on these cells. The glucose that enters the fat cells in this manner is converted into triglycerides (via the same metabolic pathways as are used by the liver) and then stored in those fat cells together with the VLDL-derived triglycerides that were made in the liver. Muscle cells also take glucose up through insulin-sensitive GLUT4 glucose channels and convert it into muscle glycogen.

A fall in blood glucose causes insulin secretion from the beta cells to be stopped, and glucagon to be secreted from the alpha cells into the blood. This inhibits the uptake of glucose from the blood by the liver, fats cells, and muscle. Instead, the liver is strongly stimulated to hydrolyze glycogen to release glucose (through glycogenolysis) and non-carbohydrate sources (such as lactate and de-aminated amino acids) using gluconeogenesis. The glucose thus produced is discharged into the blood, correcting the detected error (hypoglycemia). The glycogen stored in muscles remains in the muscles and is only broken down during exercise, to glucose-6-phosphate, and thence to pyruvate to be fed into the citric acid cycle or turned into lactate. It is only the lactate and the waste products of the citric acid cycle that are returned to the blood. The liver can take up only the lactate, and by the process of energy-consuming gluconeogenesis, convert it back to glucose.

Changes in the levels of oxygen, carbon dioxide, and plasma pH are sent to the respiratory center in the brainstem where they are regulated. The partial pressure of oxygen and carbon dioxide in the arterial blood is monitored by the peripheral chemoreceptors (PNS) in the carotid artery and aortic arch. A change in the partial pressure of carbon dioxide is detected as altered pH in the cerebrospinal fluid by central chemoreceptors (CNS) in the medulla oblongata of the brainstem. Information from these sets of sensors is sent to the respiratory center, which activates the effector organs—the diaphragm and other muscles of respiration. An increased level of carbon dioxide in the blood, or a decreased level of oxygen, will result in a deeper breathing pattern and increased respiratory rate to bring the blood gases back to equilibrium. Too little carbon dioxide, and, to a lesser extent, too much oxygen in the blood can temporarily halt breathing, a condition known as apnea, which freedivers use to prolong the time they can stay underwater.

The partial pressure of carbon dioxide is more of a deciding factor in the monitoring of pH. However, at high altitude (above 2500 m), the monitoring of the partial pressure of oxygen takes priority, and hyperventilation keeps the oxygen level constant. With the lower level of carbon dioxide, to keep the pH at 7.4, the kidneys secrete hydrogen ions into the blood and excrete bicarbonate into the urine. This is important in the acclimatization to high altitude.

The kidneys measure the oxygen content rather than the partial pressure of oxygen in the arterial blood. When the oxygen content of the blood is chronically low, oxygen-sensitive cells secrete erythropoietin (EPO) into the blood. The effector tissue is the red bone marrow, which produces red blood cells (RBCs, erythrocytes). The increase in RBCs leads to an increased hematocrit in the blood and the subsequent increase in hemoglobin that increases the oxygen-carrying capacity. This is the mechanism whereby high-altitude dwellers have higher hematocrits than sea-level residents, and also why persons with pulmonary insufficiency or right-to-left shunts in the heart (through which venous blood by-passes the lungs and goes directly into the systemic circulation) have similarly high hematocrits.

Regardless of the partial pressure of oxygen in the blood, the amount of oxygen that can be carried depends on the hemoglobin content. The partial pressure of oxygen may be sufficient, for example, in anemia, but the hemoglobin content will be insufficient and, subsequently, as will be the oxygen content. Given enough supply of iron, vitamin B₁₂, and folic acid, EPO can stimulate RBC production, and hemoglobin and oxygen content restored to normal.

The brain can regulate blood flow over a range of blood pressure values by vasoconstriction and vasodilation of the arteries. High-pressure receptors called baroreceptors in the walls of the aortic arch and carotid sinus (at the beginning of the internal carotid artery) monitor the arterial blood pressure. Rising pressure is detected when the walls of the arteries stretch due to an increase in blood volume. This causes heart muscle cells to secrete the hormone atrial natriuretic peptide (ANP) into the blood. This acts on the kidneys to inhibit the secretion of renin and aldosterone, causing the release of sodium and water into the urine, thereby reducing the blood volume. This information is then conveyed, via afferent nerve fibers, to the solitary nucleus in the medulla oblongata. From here, motor nerves belonging to the autonomic nervous system are stimulated to influence the activity of chiefly the heart and the smallest diameter arteries, called arterioles. The arterioles are the main resistance vessels in the arterial tree, and small changes in diameter cause large changes in the resistance to flow through them. When the arterial blood pressure rises, the arterioles are stimulated to dilate, making it easier for blood to leave the arteries, thus deflating them, and bringing the blood pressure down, back to normal. At the same time, the heart is stimulated via cholinergic parasympathetic nerves to beat more slowly (called bradycardia), ensuring that the inflow of blood into the arteries is reduced, thus adding to the reduction in pressure, and correction of the original error.

Low pressure in the arteries, causes the opposite reflex of constriction of the arterioles and a speeding up of the heart rate (tachycardia). If the drop in blood pressure is very rapid or excessive, the medulla oblongata stimulates the adrenal medulla, via “preganglionic” sympathetic nerves, to secrete epinephrine (adrenaline) into the blood. This hormone enhances the tachycardia and causes severe vasoconstriction of the arterioles to all but the essential organs in the body (especially the heart, lungs, and brain). These reactions usually correct the low arterial blood pressure (hypotension) very effectively.

The plasma ionized calcium (Ca²⁺) concentration is very tightly controlled by a pair of homeostatic mechanisms. The sensor for the first one is situated in the parathyroid glands, where the chief cells sense the Ca²⁺ level by means of specialized calcium receptors in their membranes. The sensors for the second are the parafollicular cells in the thyroid gland. The parathyroid chief cells secrete parathyroid hormone (PTH) in response to a fall in the plasma ionized calcium level; the parafollicular cells of the thyroid gland secrete calcitonin in response to a rise in the plasma ionized calcium level.

The effector organs of the first homeostatic mechanism are the bones, the kidney, and, via a hormone released into the blood by the kidney in response to high PTH levels in the blood, the duodenum, and jejunum. Parathyroid hormone (in high concentrations in the blood) causes bone resorption, releasing calcium into the plasma. This is a very rapid action that can correct threatening hypocalcemia within minutes. High PTH concentrations cause the excretion of phosphate ions via the urine. Since phosphates combine with calcium ions to form insoluble salts, a decrease in the level of phosphates in the blood releases free calcium ions into the plasma ionized calcium pool. PTH has a second action on the kidneys. It stimulates the manufacture and release, by the kidneys, of calcitriol into the blood. This steroid hormone acts on the epithelial cells of the upper small intestine, increasing their capacity to absorb calcium from the gut contents into the blood.

The second homeostatic mechanism, with its sensors in the thyroid gland, releases calcitonin into the blood when the blood ionized calcium rises. This hormone acts primarily on bone, causing the rapid removal of calcium from the blood and depositing it, in an insoluble form, in the bones.

The two homeostatic mechanisms working through PTH on the one hand, and calcitonin on the other can very rapidly correct any impending error in the plasma ionized calcium level by either removing calcium from the blood and depositing it in the skeleton, or by removing calcium from it. The skeleton acts as an extremely large calcium store (about 1 kg) compared with the plasma calcium store (about 180 mg). Longer-term regulation occurs through calcium absorption or loss from the gut.

The homeostatic mechanism which controls the plasma sodium concentration is rather complex. The sensor is situated in the juxtaglomerular apparatus of kidneys, which indirectly senses the plasma sodium concentration. Instead of measuring it directly in the blood flowing past the juxtaglomerular cells, these cells respond to the sodium concentration in the renal tubular fluid after it has already undergone a certain amount of modification in the proximal convoluted tubule and loop of Henle. These cells also respond to the rate of blood flow through the juxtaglomerular apparatus, which, under normal circumstances, is directly proportional to the arterial blood pressure, making this tissue an ancillary arterial blood pressure sensor.

In response to a lowering of the plasma sodium concentration, or to a fall in the arterial blood pressure, the juxtaglomerular cells release renin into the blood. Renin is an enzyme that cleaves a decapeptide from a plasma α-2-globulin called angiotensinogen. This decapeptide, with no known biological activity, is known as angiotensin I. However, when the blood circulates through the lungs, a pulmonary capillary endothelial enzyme called angiotensin-converting enzyme (ACE) cleaves further two amino acids from angiotensin I to form an octapeptide known as angiotensin II, which acts on the adrenal cortex, causing the release into the blood of the steroid hormone, aldosterone. Angiotensin II also acts on the smooth muscle in the walls of the arterioles causing these small diameter vessels to constrict, thereby restricting the outflow of blood from the arterial tree, causing the arterial blood pressure to rise. This, therefore, reinforces the measures described above, which defend the arterial blood pressure against changes, especially hypotension.

The angiotensin II-stimulated aldosterone released from the zona glomerulosa of the adrenal glands has an effect on particularly the epithelial cells of the distal convoluted tubules and collecting ducts of the kidneys. Here it causes the reabsorption of sodium ions from the renal tubular fluid in exchange for potassium ions that are secreted from the blood plasma into the tubular fluid to exit the body via the urine. The reabsorption of sodium ions from the renal tubular fluid halts further sodium ion losses from the body and, therefore, preventing the worsening of hyponatremia. The hyponatremia can only be corrected by the consumption of salt in the diet.

When the plasma sodium ion concentration is higher than normal (hypernatremia), the release of renin from the juxtaglomerular apparatus is halted, ceasing the production of angiotensin II, and its consequent aldosterone-release into the blood. The kidneys respond by excreting sodium ions into the urine, thereby normalizing the plasma sodium ion concentration. The low angiotensin II levels in the blood lower the arterial blood pressure as an inevitable concomitant response.

The reabsorption of sodium ions from the tubular fluid as a result of high aldosterone levels in the blood does not, of itself, cause renal tubular water to be returned to the blood from the distal convoluted tubules or collecting ducts. This is because sodium is reabsorbed in exchange for potassium and, therefore, causes only a modest change in the osmotic gradient between the blood and the tubular fluid. Furthermore, the epithelium of the distal convoluted tubules and collecting ducts is impermeable to water in the absence of antidiuretic hormone (ADH) in the blood. ADH is part of the control of fluid balance. Its levels in the blood vary with the osmolality of the plasma, which is measured in the hypothalamus of the brain. Aldosterone's action on the kidney tubules prevents sodium loss to the extracellular fluid (ECF). So, there is no change in the osmolality of the ECF and, therefore, no change in the ADH concentration of the plasma. However, low aldosterone levels cause a loss of sodium ions from the ECF, which could potentially cause a change in extracellular osmolality and therefore of ADH levels in the blood.

High potassium concentrations in the plasma cause depolarization of the zona glomerulosa cells' membranes in the outer layer of the adrenal cortex. This causes the release of aldosterone into the blood. Aldosterone acts primarily on the distal convoluted tubules and collecting ducts of the kidneys, stimulating the excretion of potassium ions into the urine. It does so, however, by activating the basolateral Na⁺/K⁺ pumps of the tubular epithelial cells. These sodium/potassium exchangers pump three sodium ions out of the cell, into the interstitial fluid and two potassium ions into the cell from the interstitial fluid. This creates an ionic concentration gradient which results in the reabsorption of sodium (Na⁺) ions from the tubular fluid into the blood, and secreting potassium (K⁺) ions from the blood into the urine (lumen of collecting duct).

The total amount of water in the body needs to be kept in balance. Fluid balance involves keeping the fluid volume stabilized, and also keeping the levels of electrolytes in the extracellular fluid stable. Fluid balance is maintained by the process of osmoregulation and by behavior. Osmotic pressure is detected by osmoreceptors in the median preoptic nucleus in the hypothalamus. Measurement of the plasma osmolality to give an indication of the water content of the body, relies on the fact that water losses from the body, (through unavoidable water loss through the skin which is not entirely waterproof and therefore always slightly moist, water vapor in the exhaled air, sweating, vomiting, normal feces and especially diarrhea) are all hypotonic, meaning that they are less salty than the body fluids (compare, for instance, the taste of saliva with that of tears. The latter has almost the same salt content as the extracellular fluid, whereas the former is hypotonic with respect to the plasma. Saliva does not taste salty, whereas tears are decidedly salty). Nearly all normal and abnormal losses of body water, therefore, cause the extracellular fluid to become hypertonic. Conversely, excessive fluid intake dilutes the extracellular fluid causing the hypothalamus to register hypotonic hyponatremia conditions.

When the hypothalamus detects a hypertonic extracellular environment, it causes the secretion of an antidiuretic hormone (ADH) called vasopressin, which acts on the effector organ, which in this case is the kidney. The effect of vasopressin on the kidney tubules is to reabsorb water from the distal convoluted tubules and collecting ducts, thus preventing aggravation of the water loss via the urine. The hypothalamus simultaneously stimulates the nearby thirst center causing an almost irresistible (if the hypertonicity is severe enough) urge to drink water. The cessation of urine flow prevents the hypovolemia and hypertonicity from getting worse; the drinking of water corrects the defect.

Hypo-osmolality results in very low plasma ADH levels. This results in the inhibition of water reabsorption from the kidney tubules, causing high volumes of very dilute urine to be excreted, thus getting rid of the excess water in the body.

Urinary water loss, when the body water homeostat is intact, is a compensatory water loss, correcting any water excess in the body. However, since the kidneys cannot generate water, the thirst reflex is the all-important second effector mechanism of the body water homeostat, correcting any water deficit in the body.

The plasma pH can be altered by respiratory changes in the partial pressure of carbon dioxide; or altered by metabolic changes in the carbonic acid to bicarbonate ion ratio. The bicarbonate buffer system regulates the ratio of carbonic acid to bicarbonate to be equal to 1:20, at which ratio the blood pH is 7.4 (as explained in the Henderson-Hasselbalch equation). A change in the plasma pH gives an acid-base imbalance. In acid-base homeostasis, there are two mechanisms that can help regulate the pH. Respiratory compensation a mechanism of the respiratory center, adjusts the partial pressure of carbon dioxide by changing the rate and depth of breathing, to bring the pH back to normal. The partial pressure of carbon dioxide also determines the concentration of carbonic acid and the bicarbonate buffer system can also come into play. Renal compensation can help the bicarbonate buffer system. The sensor for the plasma bicarbonate concentration is not known for certain. It is very probable that the renal tubular cells of the distal convoluted tubules are themselves sensitive to the pH of the plasma. The metabolism of these cells produces carbon dioxide, which is rapidly converted to hydrogen and bicarbonate through the action of carbonic anhydrase. When the ECF pH falls (becoming more acidic) the renal tubular cells excrete hydrogen ions into the tubular fluid to leave the body via urine. Bicarbonate ions are simultaneously secreted into the blood that decreases the carbonic acid and, consequently, raises the plasma pH. The converse happens when the plasma pH rises above normal: bicarbonate ions are excreted into the urine, and hydrogen ions released into the plasma.

When hydrogen ions are excreted into the urine, and bicarbonate into the blood, the latter combines with the excess hydrogen ions in the plasma that stimulated the kidneys to perform this operation. The resulting reaction in the plasma is the formation of carbonic acid which is in equilibrium with the plasma partial pressure of carbon dioxide. This is tightly regulated to ensure that there is no excessive build-up of carbonic acid or bicarbonate. The overall effect is, therefore, that hydrogen ions are lost in the urine when the pH of the plasma falls. The concomitant rise in the plasma bicarbonate mops up the increased hydrogen ions (caused by the fall in plasma pH) and the resulting excess carbonic acid is disposed of in the lungs as carbon dioxide. This restores the normal ratio between bicarbonate and the partial pressure of carbon dioxide and therefore the plasma pH. The converse happens when a high plasma pH stimulates the kidneys to secrete hydrogen ions into the blood and to excrete bicarbonate into the urine. The hydrogen ions combine with the excess bicarbonate ions in the plasma, once again forming an excess of carbonic acid which can be exhaled, as carbon dioxide, in the lungs, keeping the plasma bicarbonate ion concentration, the partial pressure of carbon dioxide and, therefore, the plasma pH, constant.

Cerebrospinal fluid (CSF) allows for regulation of the distribution of substances between cells of the brain and neuroendocrine factors, to which slight changes can cause problems or damage to the nervous system. For example, high glycine concentration disrupts temperature and blood pressure control, and high CSF pH causes dizziness and syncope.

Inhibitory neurons in the central nervous system play a homeostatic role in the balance of neuronal activity between excitation and inhibition. Inhibitory neurons using GABA, make compensating changes in the neuronal networks preventing runaway levels of excitation. An imbalance between excitation and inhibition is seen to be implicated in a number of neuropsychiatric disorders.

The neuroendocrine system is the mechanism by which the hypothalamus maintains homeostasis, regulating metabolism, reproduction, eating and drinking behavior, energy utilization, osmolarity, and blood pressure.

The regulation of metabolism is carried out by hypothalamic interconnections to other glands. [64] Three endocrine glands of the hypothalamic-pituitary-gonadal axis (HPG axis) often work together and have important regulatory functions. Two other regulatory endocrine axes are the hypothalamic-pituitary-adrenal axis (HPA axis) and the hypothalamic-pituitary-thyroid axis (HPT axis).

The liver also has many regulatory functions of the metabolism. An important function is the production and control of bile acids. Too much bile acid can be toxic to cells, and its synthesis can be inhibited by activation of FXR a nuclear receptor.

At the cellular level, homeostasis is carried out by several mechanisms, including transcriptional regulation that can alter the activity of genes in response to changes.

The amount of energy taken in through nutrition needs to match the amount of energy used. To achieve energy, homeostasis appetite is regulated by two hormones, ghrelin, and leptin. Ghrelin stimulates hunger, and the intake of food and leptin acts to signal satiety (fullness).

Many diseases are the result of a homeostatic failure. Almost any homeostatic component can malfunction either as a result of an inherited defect, an inborn error of metabolism, or an acquired disease. Some homeostatic mechanisms have inbuilt redundancies, which ensures that life is not immediately threatened if a component malfunctions; but sometimes a homeostatic malfunction can result in serious disease, which can be fatal if not treated.

A well-known example of a homeostatic failure is shown in type 1 diabetes mellitus. In this case, blood sugar regulation is unable to function because the beta cells of the pancreatic islets are destroyed and cannot produce the necessary insulin. The blood sugar rises in a condition known as hyperglycemia.

The plasma ionized calcium homeostasis can be disrupted by the constant, unchanging, over-production of parathyroid hormone by a parathyroid adenoma resulting in the typically features of hyperparathyroidism, namely high plasma ionized Ca², levels and the resorption of bone, which can lead to spontaneous fractures. The abnormally high plasma ionized calcium concentrations cause conformational changes in many cell-surface proteins (especially ion channels and hormone or neurotransmitter receptors) giving rise to lethargy, muscle weakness, anorexia, constipation, and labile emotions.

Roughly 60% of the mass of the body is water, and despite wide variation in the amount of water taken in each day, body water content remains incredibly stable. Such precise control of body water and solute concentrations is a function of several hormones acting on both the kidneys and vascular system, but the antidiuretic hormone ADH plays a key role in this process. The body water homeostasis can be compromised by the inability to secrete ADH in response to even the normal daily water losses via the exhaled air, the feces, and insensible sweating. On receiving a zero blood ADH signal, the kidneys produce huge unchanging volumes of very dilute urine, causing dehydration and death if not treated.

Caloric burn-rate is newly-discovered homeostasis kept nearly constant regardless of the physical activities of the person. Studies show that traditional hunter-gatherers, who lead physically hard lives, burn the same number of calories as the sedentary population in the U.S. with access to modern conveniences. Human energy expenditure is tightly constrained. See “The Exercise Paradox,” Scientific American 316, 2, 26-31 (February 2017) (doi:10.1038/scientificamerican0217-26). As organisms age, the efficiency of their control systems becomes reduced. The inefficiencies gradually result in an unstable internal environment that increases the risk of illness, and leads to the physical changes associated with aging. Various chronic diseases are kept under control by homeostatic compensation, which masks a problem by compensating for it in another way. However, the compensating mechanisms eventually wear out or are disrupted by a new complicating factor (such as the advent of a concurrent acute viral infection), which sends the body reeling through a new cascade of events. Such decompensation unmasks the underlying disease, worsening its symptoms. Common examples include decompensated heart failure, kidney failure, and liver failure.

Other biological systems have characteristics of interest that may be measured. For example, excitability of muscles, neurons, and other tissues is dependent on ion balance as well as membrane receptors. In some cases, symptoms may directly arise from a homeostatic effect, while in others, a homeostatically-controlled system may be assessed to provide information about the variables that control that system, and to infer corresponding effects on biological systems that are less readily interrogated. For example, an electrical stimulation of a particular voluntary muscle may provide direct information on excitability, that may be related to toxic effects, ion imbalances, exercise, and the like, for that muscle. However, the same causes of alteration in the excitability of a voluntary muscle may be global for the organism, and thus a muscle excitability test may provide information about the state of inaccessible muscles, as well as other tissues that have similar characteristics or are effected by the same perturbations. Likewise, peripheral edema may reflect various causes, such as ion imbalances, serum albumin, lymphatic blockage, etc.

Predictive homeostasis is an anticipatory response to an expected challenge in the future, such as the stimulation of insulin secretion by gut hormones which enter the blood in response to a meal. This insulin secretion occurs before the blood sugar level rises, lowering the blood sugar level in anticipation of a large influx into the blood of glucose resulting from the digestion of carbohydrates in the gut. Such anticipatory reactions are open-loop systems which are based, essentially, on “guesswork” and are not self-correcting. Anticipatory responses always require a closed-loop negative feedback system to correct the ‘over-shoots’ and ‘under-shoots,’ to which the anticipatory systems are prone.

### REFERENCES

Alberts, Bruce (2002). Molecular biology of the cell (4th ed.). New York [u.a.]: Garland. pp. 1292-1293. ISBN 978-0-8153-4072-0.
Alon, Uri. “An Introduction to Systems Biology: Design Principles of Biological Circuits.” Chapman and Hall/CRC (2006).
Andersen, Kim E., and Malene Hojbjerre. “A population-based Bayesian approach to the minimal model of glucose and insulin homeostasis.” Statistics in Medicine 24, no. 15 (2005): 2381-2400.
Andrews, Burton W., and Pablo A. Iglesias. “Control engineering and systems biology.” In Mathematical Methods for Robust and Nonlinear Control, pp. 267-288. Springer, London, 2007.
Antonelli, Peter L., and Vlastimil Krivan. “Fuzzy differential inclusions as substitutes for stochastic differential equations in population biology.” Open Systems & Information Dynamics 1, no. 2 (1992): 217-232.
Armstrong C M, Cota G (March 1999). “Calcium block of Na+ channels and its effect on closing rate”. Proceedings of the National Academy of Sciences of the United States of America. 96(7): 4154-7. Bibcode:1999PNAS . . . 96.4154A. doi:10.1073/pnas.96.7.4154. PMC 22436. PMID 10097179.
Aronoff, Stephen L.; Berkowitz, Kathy; Shreiner, Barb; Want, Laura (1 Jul. 2004). “Glucose Metabolism and Regulation: Beyond Insulin and Glucagon”. Diabetes Spectrum. 17 (3): 183-190. doi:10.2337/diaspect.17.3.183. ISSN 1040-9165.
Bauer J H, Gauntner W C (March 1979). “Effect of potassium chloride on plasma renin activity and plasma aldosterone during sodium restriction in normal man”. Kidney Int. 15 (3): 286-93. doi:10.1038/ki.1979.37. PMID 513492.
Bhagavan, N. V. (2002). Medical biochemistry (4th ed.). Academic Press. p. 499. ISBN 978-0-12-095440-7.
Boron W F, Boulpaep E L (2009). Medical physiology: a cellular and molecular approach (2nd Int. ed.). Philadelphia, PA: Saunders/Elsevier. ISBN 9781416031154.
Bose, Biplab. “Systems biology: A biologist's viewpoint.” Progress in biophysics and molecular biology 113, no. 3 (2013): 358-368.
Brini M, Ottolini D, Call T, Carafoli E (2013). “Chapter 4. Calcium in Health and Disease”. In Sigel A, Helmut R K (eds.). Interrelations between Essential Metal Ions and Human Diseases. Metal Ions in Life Sciences. 13. Springer. pp. 81-137. doi:10.1007/978-94-007-7500-8_4. ISBN 978-94-007-7499-5. PMID 24470090.
Campbell, Neil A. (1990). Biology (2nd ed.). Redwood City, California: The Benjamin/Cummings Publishing Company. pp. 897-898. ISBN 978-0-8053-1800-5.
Cannon, W. B. (1926). “Physiological regulation of normal states: some tentative postulates concerning biological homeostatics”. In A. Pettit (ed.). A Charles Riches amis, ses collègues, ses élèves (in French). Paris: Les Éditions Médicales. p. 91.
Cannon, W. B. (1932). The Wisdom of the Body. New York: W. W. Norton. pp. 177-201.
Chifman, J., A. Kniss, P. Neupane, I. Williams, B. Leung, Z. Deng, P. Mendes et al. “The core control system of intracellular iron homeostasis: a mathematical model.” Journal of theoretical biology 300 (2012): 91-99.
Conrad, Matthias, Christian Hubold, Bernd Fischer, and Achim Peters. “Modeling the hypothalamus-pituitary-adrenal system: homeostasis by interacting positive and negative feedback.” Journal of biological physics 35, no. 2 (2009): 149-162.
De Gaetano, Andrea, Domenico Di Martino, Alfredo Germani, and Costanzo Manes. “Mathematical models and state observation of the glucose-insulin homeostasis.” In IFIP Conference on System Modeling and Optimization, pp. 281-294. Springer, Boston, MA, 2003.
DiStefano III, Joseph. “Dynamic Systems Biology Modeling and Simulation.” Academic Press (2015).
El-Shal, Shendy M. “Digital modelling and robust control of the glucose homeostasis system.” International Journal of Systems Science 20, no. 4 (1989): 575-586.
El-Shal, Shendy M. “Microcomputer-based robust digital control for the glucoregulatory system.” International journal of systems science 22, no. 7 (1991): 1279-1293.
El-Shal, Shendy M., and Magdi S. Mahmoud. “Microcomputer-based robust control for slow time-delay processes: pole placement approach.” International Journal of Systems Science 20, no. 12 (1989): 2395-2401.
Ferrillo, Franco, Stefania Donadio, Fabrizio De Carli Phy, Sergio Garbarino, and Lino Nobili. “A model-based approach to homeostatic and ultradian aspects of nocturnal sleep structure in narcolepsy.” Sleep 30, no. 2 (2007): 157-165.
Fisher J W, Koury S, Ducey T, Mendel S (1996). “Erythropoietin production by interstitial cells of hypoxic monkey kidneys”. British Journal of Haematology. 95 (1): 27-32. doi:10.1046/j.1365-2141.1996.d01-1864.x. PMID 8857934.
Fitzsimmons, M. D., M. M. Roberts, T. G. Sherman, and A. G. Robinson. “Models of neurohypophyseal homeostasis.” American Journal of Physiology-Regulatory, Integrative and Comparative Physiology 262, no. 6 (1992): R1121-R1130.
Flores, CE; Méndez, P (2014). “Shaping inhibition: activity dependent structural plasticity of GABAergic synapses”. Frontiers in Cellular Neuroscience. 8: 327. doi:10.3389/fncel.2014.00327. PMC 4209871. PMID 25386117.
Flouris, A D (January 2011). “Functional architecture of behavioural thermoregulation”. European Journal of Applied Physiology. 111 (1): 1-8. doi:10.1007/s00421-010-1602-8. PMID 20711785.
Garmel, Gus M. (2012). “Fever in adults”. In Mahadevan, S. V.; Garmel, Gus M. (eds.). An introduction to clinical emergency medicine (2nd ed.). Cambridge: Cambridge University Press. p. 375. ISBN 978-0521747769.
Germain, Ronald N. “Maintaining system homeostasis: the third law of Newtonian immunology.” Nature immunology 13, no. 10 (2012): 902.
Gilroy, Anne M.; MacPherson, Brian R.; Ross, Lawrence M. (2008). Atlas of Anatomy. Stuttgart: Thieme Medical Publishers. pp. 318, 349. ISBN 978-1-60406-062-1.
Golubitsky, Martin, and Ian Stewart. “Homeostasis with multiple inputs.” SIAM Journal on Applied Dynamical Systems 17, no. 2 (2018): 1816-1832.
Gordon, Betts, J. Anatomy and physiology. DeSaix, Peter, Johnson, Eddie., Johnson, Jody E., Korol, Oksana., Kruse, Dean H., Poe, Brandon. Houston, Texas. p. 9. ISBN 9781947172043. OCLC 1001472383.
Hall, John (2011). Guyton and Hall textbook of medical physiology (12th ed.). Philadelphia, Pa.: Saunders/bicher. pp. 4-9. ISBN 9781416045748.
Harris, N Stuart; Nelson, Sara W (16 Apr. 2008). “Altitude Illness—Cerebral Syndromes”. EMedicine Specialties>Emergency Medicine>Environmental.
Harrison, T. R. Principles of Internal Medicine (3rd ed.). New York: McGraw-Hill Book Company. pp. 170, 571-579.
Hendrickx, Diana M., Huub C J Hoefsloot, Margriet M W B Hendriks, Daniël J. Vis, André B. Canelas, Bas Teusink, and Age K. Smilde. “Inferring differences in the distribution of reaction rates across conditions.” Molecular BioSystems 8, no. 9 (2012): 2415-2423.
Hodkinson, Duncan J., Owen O'daly, Patricia A. Zunszain, Carmine M. Pariante, Vitaly Lazurenko, Fernando O. Zelaya, Matthew A. Howard, and Steven CR Williams. “Circadian and homeostatic modulation of functional connectivity and regional cerebral blood flow in humans under normal entrained conditions.” Journal of Cerebral Blood Flow & Metabolism 34, no. 9 (2014): 1493-1499.
Hu C, Rusin C G, Tan Z, Guagliardo N A, Barrett P Q (June 2012). “Zona glomerulosa cells of the mouse adrenal cortex are intrinsic electrical oscillators”. J Clin Invest. 122 (6): 2046-2053. doi:10.1172/JC161996. PMC 3966877. PMID 22546854.
Ingalls, Brian P., Tau-Mu Yi, and Pablo A. Iglesias. “12 Using Control Theory to Study Biology.” System Modeling in Cellular Biology (2004): 243.
Ingalls Brian P. “Mathematical Modeling in Systems Biology: An Introduction.” The MIT Press (2013).
Jelkmann, W. (2007). “Erythropoietin after a century of research: younger than ever”. European Journal of Haematology. 78 (3): 183-205. doi:10.1111/j.1600-0609.2007.00818.x. PMID 17253966.
Kalaany, N Y; Mangelsdorf, D J (2006). “LXRS and FXR: the yin and yang of cholesterol and fat metabolism”. Annual Review of Physiology. 68: 159-91. doi:10.1146/annurev.physiol.68.033104.152158. PMID 16460270.
Khan Academy. “Homeostasis”. Khan Academy.
Kluge, Matthew J. (2015). Fever: Its Biology, Evolution, and Function. Princeton University Press. p. 57. ISBN 9781400869831.
Koeslag, J. H.; Saunders, P. T.; Wessels, J. A. (1997). “Glucose homeostasis with infinite gain: further lessons from the Daisyworld parable?”. Journal of Endocrinology. 134: 187-192.
Koeslag, Johan H.; Saunders, Peter T.; Terblanche, Elmarie (2003). “Topical Review: A reappraisal of the blood glucose homeostat which comprehensively explains the type 2 diabetes-syndrome X complex”. Journal of Physiology. 549 (Pt 2): 333-346. doi:10.1113/jphysiol.2002.037895. PMC 2342944. PMID 12717005.
Koeslag, Johan H.; Saunders, Peter T.; Wessels, Jabus A. (1999). “The chromogranins and counter-regulatory hormones: do they make homeostatic sense?”. Journal of Physiology. 517 (3): 643-649. doi:10.1111/j.1469-7793.1999.0643s.x. PMC 2269385. PMID 10358106.
LeDuc, Philip R., William C. Messner, and John P. Wikswo. “How do control-based approaches enter into biology?.” Annual Review of Biomedical Engineering 13 (2011): 369-396.
Linas S L, Peterson L N, Anderson R J, Aisenbrey G A, Simon F R, Berl T (June 1979). “Mechanism of renal potassium conservation in the rat”. Kidney International. 15 (6): 601-11. doi:10.1038/ki.1979.79. PMID 222934.
Longo, Giuseppe; Montévil, Maël (2014). Perspectives on Organisms. Lecture Notes in Morphogenesis. Springer. doi:10.1007/978-3-642-35938-5. ISBN 978-3-642-35937-8.
Lovelock, James (1991). Healing Gaia: Practical Medicine for the Planet. New York: Harmony Books. ISBN 978-0-517-57848-3.
Lutaif, N. A., R. Palazzo Jr, and J. A. R. Gontijo. “Early detection of metabolic and energy disorders by thermal time series stochastic complexity analysis.” Brazilian Journal of Medical and Biological Research 47, no. 1 (2014): 70-79.
Mamontov, Eugen. “Modelling homeorhesis by ordinary differential equations.” Mathematical and computer modelling 45, no. 5-6 (2007): 694-707.
Mansour, N. E., and D. A. Linkens. “Pole-assignment self-tuning control of blood pressure in postoperative patients: a simulation study.” In IEE Proceedings D (Control Theory and Applications), vol. 136, no. 1, pp. 1-11. IET Digital Library, 1989.
Marder, Eve, and Astrid A. Prinz. “Modeling stability in neuron and network function: the role of activity in homeostasis.” Bioessays 24, no. 12 (2002): 1145-1154.
Marieb E N, Hoehn K N (2009). Essentials of Human Anatomy & Physiology (9th ed.). San Francisco: Pearson/Benjamin Cummings. ISBN 978-0321513427.
Martin, Elizabeth (2008). A dictionary of biology (6th ed.). Oxford: Oxford University Press. pp. 315-316. ISBN 978-0-19-920462-5.
Mavelli, Fabio, and Pasquale Stano. “Kinetic models for autopoietic chemical systems: the role of fluctuations in a homeostatic regime.” Physical Biology 7, no. 1 (2010): 016010.
Milsum, J. H. (1966). Biological control systems analysis. New York: McGraw-Hill.
Mohler, R. “Biological modeling with variable compartmental structure.” IEEE Transactions on Automatic Control 19, no. 6 (1974): 922-926.
Ni, Xiao Yu, Tonnod Drengstig, and Peter Ruoff. “The control of the controller: molecular mechanisms for robust perfect adaptation and temperature compensation.” Biophysical journal 97, no. 5 (2009): 1244-1253.
Norwich, Kenneth H., and Raymond Reiter. “Homeostatic control of thyroxin concentration expressed by a set of linear differential equations.” The Bulletin of mathematical biophysics 27, no. 2 (1965): 133.
Palmer, L G; Frindt, G (2000). “Aldosterone and potassium secretion by the cortical collecting duct”. Kidney International. 57 (4): 1324-8. doi:10.1046/j.1523-1755.2000.00970.x. PMID 10760062.
Pattaranit, Ratchada, and Hugo Antonius Van Den Berg. “Mathematical models of energy homeostasis.” Journal of The Royal Society Interface 5, no. 27 (2008): 1119-1135.
Peacock, Andrew J (17 Oct. 1998). “Oxygen at high altitude”. British Medical Journal. 317 (7165): 1063-1066. doi:10.1136/bmj.317.7165.1063. PMC 1114067. PMID 9774298.
Peters, Achim, Matthias Conrad, Christian Hubold, Ulrich Schweiger, Bernd Fischer, and Horst Lorenz Fehm. “The principle of homeostasis in the hypothalamus-pituitary-adrenal system: new insight from positive feedback.” American Journal of Physiology-Regulatory, Integrative and Comparative Physiology 293, no. 1 (2007): R83-R98.
Peterson, Mark C., and Matthew M. Riggs. “A physiologically based mathematical model of integrated calcium homeostasis and bone remodeling.” Bone 46, no. 1 (2010): 49-63.
Pocock, Gillian; Richards, Christopher D. (2006). Human physiology: the basis of medicine (3rd ed.). Oxford: Oxford University Press. ISBN 978-0-19-856878-0.
Poh, C. L., L. C. Cui, and R. I. Kitney. “Modeling biological systems in Laplace Domain for Synthetic Biology Design.” In World Congress on Medical Physics and Biomedical Engineering, Sep. 7-12, 2009, Munich, Germany, pp. 1377-1380. Springer, Berlin, Heidelberg, 2009.
Preston, Richard A.; Materson, B. J.; Reda, D. J.; Williams, D. W.; Hamburger, R. J.; Cushman, W. C.; Anderson, R. J. (1998). “JAMA Article Jan 2012”. JAMA. 280 (13): 1168-72. doi:10.1001/jama.280.13.1168. PMID 9777817.
Purves, Dale (2011). Neuroscience (5th ed.). Sunderland, Mass.: Sinauer. p. 458. ISBN 978-0-87893-695-3.
Rasmussen, Howard. “Cellular calcium homeostasis and the calcium messenger system.” In Seminars in liver disease, vol. 5, no. 02, pp. 110-121. Thieme Medical Publishers, Inc., 1985.
Riggs, D. S. (1970). Control theory and physiological feedback mechanisms. Baltimore: Williams & Wilkins.
Rucker, R. (1987). Mind tools: the mathematics of information. Harmondsworth: Penguin Books. pp. 25-30.
Ruoff, Peter. “General homeostasis in period- and temperature-compensated chemical clock mutants formed by random selection conditions.” Naturwissenschaften 81, no. 10 (1994): 456-459.
Sakka, L.; Coll, G.; Chazal, J. (December 2011). “Anatomy and physiology of cerebrospinal fluid”. European Annals of Otorhinolaryngology, Head and Neck Diseases. 128 (6): 309-316. doi:10.1016/j.anorl.2011.03.002. PMID 22100360.
Saladin, Kenneth (2012). Anatomy and Physiology (6th ed.). McGraw Hill. pp. 519-20.
Schmidt-Nielsen K (1981). “Countercurrent systems in animals”. Scientific American. 244 (5): 118-28. Bibcode:1981SciAm.244e.118S. doi:10.1038/scientificamerican0581-118. PMID 7233149.
Shannon, Claude E.; Weaver, Warren (1963). The mathematical theory of communication (4th print. ed.). Urbana: University of Illinois Press. ISBN 978-0252725487.
Sievanen, H. “Hormonal influences on the muscle-bone feedback system: a perspective.” Journal of Musculoskeletal and Neuronal Interactions 5, no. 3 (2005): 255.
Singh, Prabhleen, and Scott C. Thomson. “Renal homeostasis and tubuloglomerular feedback.” Current opinion in nephrology and hypertension 19, no. 1 (2010): 59-64.
Smith, Gerard P. (2008). “Unacknowledged contributions of Pavlov and Barcroft to Cannon's theory of homeostasis”. Appetite. 51 (3): 428-432. doi:10.1016/j.appet.2008.07.003.
Smith, James M D, James A. Maas, Philip C. Garnsworthy, Markus R. Owen, Stephen Coombes, Tahir S. Pillay, David A. Barrett, and Michael E. Symonds. “Mathematical modeling of glucose homeostasis and its relationship with energy balance and body fat.” Obesity 17, no. 4 (2009): 632.
Spencer, Laci (2015). Flotation: A Guide for Sensory Deprivation, Relaxation, & Isolation Tanks. Lulu.com. p. 29. ISBN 978-1329173750.
Spencer, Laci (29 May 2015). Flotation: A Guide for Sensory Deprivation, Relaxation, & Isolation Tanks. Lulu.com. ISBN 9781329173750.
Spyer, K M; Gourine, A V (12 Sep. 2009). “Chemosensory pathways in the brainstem controlling cardiorespiratory activity”. Philosophical Transactions of the Royal Society of London. Series B, Biological Sciences. 364 (1529): 2603-10. doi:10.1098/rstb.2009.0082. PMC 2865116. PMID 19651660.
Standring, Susan (7 Aug. 2015). Gray's anatomy: the anatomical basis of clinical practice. (41st ed.). pp. 141, 151-152. ISBN 9780702068515. OCLC 920806541.
Stryer, Lubert (1995). “Vitamin D is derived from cholesterol by the ring-splitting action of light.”. In: Biochemistry (4th ed.). New York: W.H. Freeman and Company. p. 707. ISBN 0 7167 2009 4.
Stryer, Lubert (1995). Biochemistry (4th ed.). New York: W.H. Freeman and Company. pp. 164, 773-774. ISBN 0 7167 2009 4.
Stuart, I. R. (2011). Human physiology (12th ed.). New York: McGraw-Hill. p. 667.
Sütterlin, Thomas, Simone Huber, Hartmut Dickhaus, and Niels Grabe. “Modeling multi-cellular behavior in epidermal tissue homeostasis via finite state machines in multi-agent systems.” Bioinformatics 25, no. 16 (2009): 2057-2063.
Swedan, Nadya Gabriele (2001). Women's Sports Medicine and Rehabilitation. Lippincott Williams & Wilkins. p. 149. ISBN 978-0-8342-1731-7.
Talmage, D. W., and R. V. Talmage. “Calcium homeostasis: how bone solubility relates to all aspects of bone physiology.” Journal of Musculoskeletal and Neuronal Interactions 7, no. 2 (2007): 108.
Tansey, Etain A.; Johnson, Christopher D (2015). “Recent advances in thermoregulation”. Advances in Physiology Education. 39 (3): 139-148. doi:10.1152/advan.00126.2014. ISSN 1043-4046. PMID 26330029.
Thomas, S. Randall, Pierre Baconnier, Julie Fontecave, Jean-Pierre Francoise, Frangois Guillaud, Patrick Hannaert, Alfredo Hernandez, et al. “SAPHIR: a physiome core model of body fluid homeostasis and blood pressure regulation.” Philosophical Transactions of the Royal Society A: Mathematical, Physical and Engineering Sciences 366, no. 1878 (2008): 3175-3197.
Thorsen, Kristian, Oleg Agafonov, Christina H. Selsto, Ingunn W. Jolma, Xiao Y. Ni, Tormod Drengstig, and Peter Ruoff. “Robust concentration and frequency control in oscillatory homeostats.” PloS one 9, no. 9 (2014): e107766.
Toni, R (2004). “The neuroendocrine system: organization and homeostatic role”. Journal of Endocrinological Investigation. 27 (6 Suppl): 35-47. PMID 15481802.
Tortora, Gerard J.; Anagnostakos, Nicholas P. (1987). Principles of Anatomy and Physiology (Fifth ed.). New York: Harper & Row, Publishers. ISBN 978-0-06-350729-6.
Um, Ji Won (13 Nov. 2017). “Roles of Glial Cells in Sculpting Inhibitory Synapses and Neural Circuits”. Frontiers in Molecular Neuroscience. 10: 381. doi:10.3389/fnmol.2017.00381. PMC 5694142. PMID 29180953.
Watson, Edmund M., Michael J. Chappell, F. Ducrozet, S. M. Poucher, and James W T Yates. “A new general glucose homeostatic model using a proportional-integral-derivative controller.” Computer methods and programs in biomedicine 102, no. 2 (2011): 119-129.
Weschler, Toni (2002). Taking Charge of Your Fertility. New York: HarperCollins. pp. 52, 316, 361-362. ISBN 978-0-06-093764-5.
West, Bruce J (2006). Where Medicine Went Wrong: Rediscovering the Path to Complexity. Studies of Nonlinear Phenomena in Life Science. 11. New Jersey: World Scientific. doi:10.1142/6175. ISBN 978-981-256-883-0.
Williams G H, Dluhy R G (2008). “Chapter 336: Disorders of the Adrenal Cortex”. In Loscalzo J, Fauci A S, Braunwald E, Kasper D L, Hauser S L, Longo D L (eds.). Harrison's principles of internal medicine. New York: McGraw-Hill Medical. ISBN 978-0-07-146633-2.
Williams, Peter L.; Warwick, Roger; Dyson, Mary; Bannister, Lawrence H. (1989). Gray's Anatomy (Thirty-seventh ed.). Edinburgh: Churchill Livingstone. pp. 691-692, 791, 10011-10012. ISBN 0443 041776.
Withers, P. C. “Comparative physiology.” Saunders College, Fort Worth (1992).
Young, Andrew J; Reeves, John T. (2002). “Human Adaptation to High Terrestrial Altitude” (PDF). Medical Aspects of Harsh Environments. 2. Borden Institute, Washington, DC. CiteSeerX 10.1.1.175.3270.
Zion, Michal, and Sara Klein. “Conceptual understanding of homeostasis.” International Journal of Biology Education 2 (2015): 1-27.
Zorea, Aharon (2014). Steroids (Health and Medical Issues Today). Westport, CT: Greenwood Press. p. 10. ISBN 978-1440802997.

It is typically assumed that the underlying function describing the physiological parameter of interest over time is, or can be described by, a smooth function. This assumption is well justified, except, perhaps, as applied to electrically excitable cells when sampled well below the Nyquist rate of their action potentials. Few things in biology are quantized, and most natural biological processes are continuous analog processes that are described by smooth differentiable functions.

For example, whether we are interested in the function describing change of the body temperature (T) over time (t), T(t), or the function describing changing the level of the glucose (G) in blood, G(t), all these functions are smooth and differentiable in a sense that there is at least a first ƒ′=dƒ/dt and second derivative ƒ″=d²ƒ/dt² of these functions over time. However, in most cases, it is not possible to monitor these parameters continuously, and sampling of the physiological data is discrete. Further, compression of the data will often discretize the information, even if it is initially in a continuous form. Thus, sampled physiological data subject to analysis typically is present as a time series.

While periodic sampling facilitates the analysis, this is not required in all cases. In periodically sampled data, a parameter of interest is sampled at fixed periods spaced by a predetermined time interval, Δtᵢ, at times t∈{0, Δt₁, Δt₂, . . . , Δtₙ}.

In some cases, sampling may not always occur at precise and predictable times, i.e., the sampling interval is irregular.

There are different methods of approximating the “acceleration,” i.e., the second-order rate of change in a parameter over time. For example, the first and the second derivatives of a continuous function, which has true first and second derivatives may be determined, ƒ′=dƒ/dt and ƒ″=d²ƒ/dt². It is not possible to directly differentiate discrete sampled values. One possible solution is to fit the discrete data into a continuous function that can be differentiated, which can be accomplished by interpolation, smoothing, or any other curve-fitting technique, such as, for example, spline interpolation, polynomial interpolation or polynomial regression; using trigonometric, Gaussian, Lorentzian, Voigt and related function; ordinary and total least squares, etc.

Interpolation is a technique which is used for constructing new data points within the range of discrete data points of the discrete data retrieved from the sampling of a smooth function, which makes it a simple function and allows for the regular differentiation by taking the first and the second derivative of the interpolated function. Interpolation requires an inherent presumption that the data is oversampled, i.e., significant changes do not occur between samples, and so all significant data is captured by the samples.

Of course, the process of starting from sampled data, and interpolating, only to extract values, may be inefficient. Rather, direct analysis of the sampled data is preferred and may be more accurate, since the smoothing functions may lose information and introduce a presumption about the state of the signal between samples that is unwarranted. In many cases, the sampled data is sufficiently oversampled with respect to the physiological process that a curve may be optimally fit to a segment of the line, and the shape of the optimally fit segment will define the first and second derivatives. In other cases, the data is undersampled, and a large amount of variance will be due to physiological changes between samples. In that case, a statistical analysis is more appropriate, and short-term dynamics may be difficult to analyze. Finally, sampling may occur at a rate near a natural cycle frequency or harmonic of the underlying physiological changes, unless the sampling is synchronized with the process, and that analysis recognizes that the values between sampled datapoints are oscillatory. In the more general case, it may also difficult to analyze the data subject to aliasing, though, over longer periods, data patterns may be extracted.

There are numerous techniques for interpolating data. Approximation and interpolation differ in the sense that in approximation, all the points of the basic figure need not be located on the created figure called an approximation curve segment, whereas all the points of the basic figure are located on the created figure called interpolation curve segment.

In discrete calculus (a.k.a. finite differences), the discrete derivative of a function ƒ(n), denoted Δₙƒ(n), is defined to be ƒ(n+1)−ƒ(n). In a finite difference approximation, the first backward derivative is defined as:

$${x}_{t}^{′} = \frac{{x}_{t} - {x}_{t - 1}}{Δ t}$$

The first forward derivative is defined as:

$${x}_{t}^{′} = \frac{{x}_{t + 1} - {x}_{t}}{Δ t}$$

The first centered derivative is defined as:

$${x}_{t}^{′} = \frac{{x}_{t + 1} - {x}_{t - 1}}{2 Δ t} .$$

The second derivative is defined as:

$${x}_{t}^{″} = \frac{{x}_{t + 1} - 2 {x}_{t} + {x}_{t - 1}}{{( Δ t )}^{2}} .$$

It is preferable to use centered finite differences, when possible because it is an approximation of order 2:

$$\vert {\dot{x}}_{t} - \frac{{x}_{t + 1} - {x}_{t - 1}}{2 Δ t} \vert = 0 {( Δ t )}^{2}$$

where \dot{x} denotes the actual derivative of x at t. We assume here that there is an underlying smooth function x(⋅) such that xₜ=x(t), i.e., the data is a result of sampling from a smooth process. These formulae are derived by computing a simple function that interpolates the data (for example, a polynomial or a spline), and then differentiating that function. Thus, this approach is equivalent to the interpolation (curve-fitting) and subsequent differentiation of the resulting function.

Other methods in numerical differentiation include the five-point stencil, which gives a better approximation for the second derivative of a function ƒ(x) in one dimension f″≈(−f(x+2h)+16f(x+h)−30f(x)+16f(x−h)−f(x−2h))/(12h²

statistical methods include noise reduction techniques. Various digital filters (e.g., such as Savitzky-Golay Filter) may be applied for smoothing the data.

An alternative to interpolation is to employ an alternate mathematical function, the z-transform. The z-transform converts a discrete-time signal, which is a sequence of real or complex numbers, into a complex frequency-domain representation. It can be considered as a discrete-time equivalent of the Laplace transform. See, en.wikipedia.org/wiki/Z-transform.

A linear system can be represented in the complex frequency domain (s-domain, where s=σ+jω) using the Laplace Transform X(s), where the direct transform is:

$$L \{ x ( t ) \} = X ( s ) = {\int }_{t = 0}^{∞} x ( t ) {ε}^{- st} dt$$

and x(t) is assumed zero for t≤0.

The Inversion integral is a contour integral in the complex plane:

$${L}^{- 1} \{ X ( s ) \} = x ( t ) = \frac{1}{2 π j} {\int }_{s = σ - j ∞}^{σ + j ∞} X ( s ) {ε}^{st} ds$$

where σ is chosen such that the contour integral converges.

If we assume that x(t) is ideally sampled, where: xₙ=x(n*Tₛ)=x(t)|_{t=n*T,}, and yₙ=y(n*Tₛ)=y(t)|_{t=n*T,}, this equivalent system may be analyzed using standard analog tools to establish the z-Transform. Substituting the Sampled version of x(t) into the definition of the Laplace Transform we get L{x(t,Tₛ)}=X_{T}(s)=∫ₜ₌₀^{∞}x(t,Tₛ)ε^{-st}dt

But,

$$x ( t , {T}_{s} ) = {\sum }_{n = 0}^{∞} x ( t ) * p ( t - n * {T}_{s} )$$

(For x(t)=0 when t<0). Therefore:

$${X}_{T} ( s ) = {\int }_{t = 0}^{∞} [ {\sum }_{n = 0}^{∞} x ( n * {T}_{s} ) * δ ( t - n * {T}_{s} ) ] {ε}^{- st} dt$$

Now interchanging the order of integration and summation and using the sifting property of δ-functions:

$${X}_{T} ( s ) = {\sum }_{n = 0}^{∞} x ( n * {T}_{s} ) {\int }_{t = 0}^{∞} * δ ( t - n * {T}_{s} ) ] {ε}^{- st} dt$$

$${X}_{T} ( s ) = {\sum }_{n = 0}^{∞} x ( n * {T}_{s} ) {ε}^{- {nT}_{s} s}$$

(We are assuming that the first sample occurs at t=0+)
if we now adjust our nomenclature by letting: z=ε^{sT}, x(n*Ts)=xₙ, and

$$X ( z ) = {X}_{T} ( s ) {\vert}_{z = {ε}^{sT}}$$

$$X ( z ) = {\sum }_{n = 0}^{∞} {x}_{n} {z}^{- n} ,$$

which is the direct z-transform (one-sided; it assumes xₙ=0 for n<0).

The inversion integral is:

$${x}_{n} = \frac{1}{2 π j} X ( z ) {z}^{n - 1} dz$$

(This is a contour integral in the complex z-plane; The use of this integral can be avoided as tables can be used to invert the transform.)

To prove that these form a transform pair, we can substitute one into the other.

$${x}_{k} = \frac{1}{2 π j} {∮}_{c} [ {\sum }_{n = 0}^{∞} {x}_{n} {z}^{- n} ] {z}^{k - 1} dz$$

Now interchanging the order of summation and integration (valid if the contour followed stays in the region of convergence):

$${x}_{k} = \frac{1}{2 π j} {\sum }_{n = 0}^{∞} {x}_{n} {∮}_{c} {z}^{k - n - 1} dz$$

If “C” encloses the origin (that's where the pole is), the Cauchy Integral theorem says:

$${∮}_{c} {z}^{k - n - 1} dz = \begin{matrix}o for n \neq k \\ 2 π j for n = k\end{matrix} ,$$

and we get xₖ=xₖ Q.E.D.

The Z transform provides a frequency domain version of a discrete-time signal. Discrete-time signals are sequences, and the Z transform is defined by

$$\{ {h}_{k} \} {⟷}^{Z transform} H ( z ) = {\sum }_{- ∞}^{∞} {h}_{k} {z}^{- k} . \qquad ( 1 )$$

Consider, for example, the elementary Z transform pair

$${h}_{k} = {a}^{k} {u}_{k} {⟷}^{Z} H ( z ) = \frac{1}{1 - {az}^{- 1}} , \qquad ( 2 )$$

where uₖ is the unit step-function. The time-domain sequence hₖ and the frequency function H(z) are alternate ways of describing the same signal. In the time domain, hₖ is exponential. In the frequency domain, H(z) is rational or, by definition, the ratio of two polynomials. For discrete-time applications, we will use the representation

$$H ( z ) = {z}^{υ} \frac{B ( z )}{A ( z )} , \qquad ( 3 )$$

where B(z)=b₀+b₁z⁻⁺ . . . +bₘz^{-m}, A(z)=1+a₁z⁻¹+a₂z⁻²+ . . . +aₙz⁻ⁿ, and v is an integer. The representation is unique if we demand that the end coefficients b₀, bₘ, aₙ and an are not zero.

The poles of H(z) are the roots of the denominator polynomial A(z). At a pole, H(z) becomes infinite. The zeros of H(z) are the roots of the numerator polynomial B(z). At a zero, H(z) is zero. A pole-zero plot of H(z) simply places the poles (using the symbol x) and the zeros (using the symbol o) on the complex plane. For stability, it is necessary that the poles of H(z) be inside the unit disk, or in other words, have an absolute value less than one. The complex frequency response is computed by evaluating H(z) on the unit circle z=e^{jθ}, 0≤θ2π.

The choice of the letter ‘h’ for the above signal is commonly used for filters. In the time domain, hₖ is the unit pulse response sequence of the filter. In the frequency domain, H(z) is the transfer function of the filter. If we set z=e^{jθ}, then we get the complex frequency response function H(e^{jθ}). In fact, with z=e^{jθ}, the Z transform becomes the DTFT, or Discrete Time Fourier Transform:

$$\{ {h}_{k} \} {⟷}^{DTFT} H ( {e}^{j θ} ) = {\sum }_{- ∞}^{∞} {h}_{k} {e}^{- jk θ} . \qquad ( 4 )$$

This transform has the inversion rule

$${h}_{k} = {\int }_{- π}^{π} H ( {e}^{j θ} ) {e}^{j k θ} \frac{d θ}{2 π} . \qquad ( 5 )$$

Laplace transforms use the s plane, and frequency response is computed on the imaginary axis s=jω. The Z transform uses the z plane, and the frequency response is computed on the unit circle z=e^{jθ}. The Laplace transform is appropriate for continuous-time systems, while the Z transform is appropriate for discrete-time systems.

Suppose that we start with continuous-time signals, and sample them to get discrete-time signals. Let the sampling frequency be ƒₛ, and the sampling period be tₛ=1/ƒₛ=2π/ωₛ. Consider a signal x(t) with Laplace transform

$$X ( s ) = {\int }_{- ∞}^{∞} x ( t ) {e}^{- st} dt , \qquad ( 6 )$$

and let y^{k}=x(ktₛ) be the discrete-time signal obtained by sampling x(t). The Z Transform of the sequence of samples is

$$y ( z ) = {\sum }_{k = - ∞}^{∞} {y}_{k} {z}^{- k} . \qquad ( 7 )$$

This sum approximates the integral in equation (6), if we set

$$z = {e}^{{st}_{s}} . \qquad ( 8 )$$

This relation maps the s plane into the z plane. Using equation (8) in equation (7), we get

$${t}_{s} Y ( {e}^{{st}_{s}} ) = {t}_{s} {\sum }_{k = - ∞}^{∞} {y}_{k} {z}^{- k} = {\sum }_{k = - ∞}^{∞} {x ( {kt}_{s} )}^{- {skt}_{s}} {t}_{s} \approx {\int }_{- ∞}^{∞} x ( t ) {e}^{- st} dt = X ( s ) . \qquad ( 9 )$$

Therefore, the Z transform of y can approximate the Laplace Transform of x, and the DTFT of y can approximate the Fourier Transform of x, as in the following table:

TABLE 1

| | | |
|---|---|---|
| Laplace Transform/ | X(s) ≈ tₛY(z) | provided z = e^{st}^{ₛ}, and |
| Z Transform | | \|imag(s)\| < ωₛ/2 |
| Fourier Transform/ | X (jω) ≈ tₛY(e^{jθ}) | provided θ = ωtₛ = 2πω/ωₛ and |
| DTFT | | \|ω\| < ωₛ/2, or \|θ\| < π |

The approximation has its limits because going around the unit circle is a periodic motion. The DTFT is Y(e^{jθ}) is 2π periodic in θ=ωtₛ=2πω/ωₛ and the approximation to X(jω) is therefore periodic with period ωₛ. Because of symmetry about zero, this means that the approximation is good only to the half sampling frequency ωₛ/2, i.e., the Nyquist rate. In digital signal processing, bandwidth is limited. For greater bandwidth, one must sample faster.

There is a precise formula which relates Y(e^{jθ}) and X(jω), when yₖ=x(ktₛ), as opposed to the approximation that we have already mentioned. Whenever one samples in the time domain, then there will be aliasing in the frequency domain. The formula is this:

$${y}_{k} = x ( {kt}_{s} ) {⟷}^{DTFT} Y ( {e}^{j ω {t}_{s}} ) = \frac{1}{{t}_{s}} {\sum }_{n = - ∞}^{∞} X ( j ω - jn {ω}_{s} ) . \qquad ( 10 )$$

$$( sampling in time ) ⟷ ( aliasing in frequency )$$

When X(jω) is bandlimited to |ω|<ω/2, the formula X(jω)=tₛ·Y(e^{jωt}^{ₛ}) will hold exactly for |ω|<ωₛ/2. (This is the sampling theorem.)

If B(z) has degree equal to that of A(z), and if A(z) does not have repeated roots, then the Z transform pair for H(z)=B(z)/A(z) is

$${h}_{k} = {b}_{0} {δ}_{k} + [ {\sum }_{m - 1}^{n} {γ}_{m} {α}_{m}^{k - 1} ] {u}_{k - 1} {⟷}^{Z} H ( z ) = {b}_{0} + {\sum }_{m - 1}^{n} \frac{{γ}_{m}}{z - {α}_{m}} = {b}_{0} + {z}^{1} {\sum }_{m - 1}^{n} \frac{{γ}_{m}}{1 - {α}_{m} {z}^{- 1}} . \qquad ( 11 )$$

The right-hand side of the above is a partial fraction expansion, of H(z). Under the conditions specified, the parameters b₀, γ₁, γ₂ . . . , γₘ, α₁, α₂, . . . , αₘ can be computed.

Therefore, it is apparent that typical z-transform calculations assume periodic sampling, and the generally published analyses do not address aperiodic sampling. Further, one cannot simply interpolate the sampled signal to reduce the sampling imprecision below an error threshold, since the frequency characteristics of the function will be altered by the synthetic sampling, depending on the interpolation method. If one presumes that the sampling occurs well above the Nyquist rate, then perhaps the sampling time jitter may be ignored with small loss of accuracy, but if the underlying signal is not fully bandlimited, a simple interpolation, smoothing, or the like, is both theoretically and practically improper.

For the discrete variable case, the z-Transform of x[n] is given by

$$X ( z ) = {\sum }_{n = - ω}^{ω} x [ n ] {z}^{- n}$$

On differentiating both sides with respect to z, and then multiplying both sides by −z, we get

$$- z \frac{d}{d z} X ( z ) = {\sum }_{n = - ω}^{ω} ( n x [ n ] ) {z}^{- n} ,$$

provided it exists. This can be thought of as taking the z-Transform of nx[n]. Hence, we observe that if

$$x [ n ] {\to }^{z} X ( z ) with ROC as ‘ R ’ , then$$

$$nx [ n ] {\to }^{z} - z \frac{d}{d z} X ( z ) with ROC as ‘ R ’ .$$

Similarly, we can proceed further by differentiating again and again.

If

$$x [ n ] {↔}^{z} X ( z )$$

with ROC=R, then, because of

$$x [ n - {n}_{0} ] {↔}^{z} {z}^{- {n}_{0}} X ( z )$$

with ROC=R except for the possible addition or deletion of the origin or infinity the multiplication by z⁻ⁿ^{₀} for nₒ>0 poles will be introduced at z=0, which may cancel corresponding zeroes of X(z) at z=0. In this case, the ROC for z⁻ⁿ^{₀}X(z) equals the ROC of X(z) but with the origin deleted. Similarly, if n₀<0, z=∞ may get deleted.

It is noted that the fact that the more rigorous z-transform method does not provide a simple basis for the analysis of an aperiodically sampled discrete-time sample, the simpler method(s) inherently reflect this defect.

### Differential Equations

In a differential equation, the “order” refers to the order of the highest derivative. A linear ordinary differential equation (ODE) can be put into the following form:

$${f}_{n} ( x ) \frac{{d}^{n} y}{d {x}^{n}} + {f}_{n - 1} ( x ) \frac{{d}^{n - 1} y}{d {x}^{n - 1}} + … + {f}_{1} ( x ) \frac{d y}{d x} + {f}_{0} ( x ) y = F ( x )$$

A characteristic of linear systems is superposition, and therefore functions of orthogonal variables may be separated, i.e., a solution of an equation of form ƒ(y)dy+g(x)dx=0 is of form

$$\int f ( y ) d y + \int g ( x ) dx = C ;$$

for example,

$$q ( {x}_{i} - x ) d t = Vdx ⇒ \int \frac{d x}{{x}_{i} - x} - \int \frac{q}{V} d t = C ⇒ ln ( x - {x}_{i} ) + \frac{q}{V} t = C$$

$$⇒ x - {x}_{i} = K {e}^{- \frac{q}{V} t} ⇒ if x ( 0 ) = {x}_{0} , K = {x}_{0} - {x}_{i} ⇒ x = {x}_{i} + ( {x}_{0} - {x}_{i} ) {e}^{- \frac{q}{V} t}$$

Homogeneous equations are of form:

$$\frac{dy}{dx} = f ( \frac{y}{x} ) ,$$

and may be solved as follows:

Let

$$u = \frac{y}{x} ⇒ \frac{dy}{dx} = u + x \frac{du}{dx}$$

Substitute and separate u and x and then integrate:

$$\int \frac{dx}{x} = \int \frac{du}{f ( u ) - u} + C ,$$

and substitute u=y/x to obtain the general solution.

While the present technology typically addresses second-order differential responses, it is not so limited. n^{th}-order linear homogeneous equations with constant coefficients have the form:

$${A}_{n} \frac{{d}^{n} y}{d {x}^{n}} + {A}_{n - 1} \frac{{d}^{n - 1} y}{d {x}^{n - 1}} + … + {A}_{1} \frac{d y}{d x} + {A}_{0} y = 0 {A}_{n} , {A}_{n - 1} , … , {A}_{1} , {A}_{0}$$

are constants. These may be solved by constructing an auxiliary equation: Aₙrⁿ+Aₙ₊₁rⁿ⁻¹+ . . . +A₁r+A₀=0, and the auxiliary equation solved, e.g., for each distinct real root (rₐ): yₐ=Cₐ exp(rₐx); for each set of repeat (m times) real roots (r_{b}):

$${y}_{b} = ( {C}_{b 1} + {C}_{b 2} x + … + {C}_{b m} {x}^{m - 1} ) exp ( {r}_{b} x ) = [ {\sum }_{i = 1}^{m - 1} {C}_{bi} {x}^{i - 1} ] exp ( {r}_{b} x ) ;$$

For each pair of Complex roots (a±jb): y_{c}=(C_{c} sin bx+C′_{c} cos bx)exp(ax), with general solution: y=Σyₐ+Σy_{b}+Σy_{c}.

An n^{th} order linear nonhomogeneous equation with constant coefficients has the form:

$${A}_{n} \frac{{d}^{n} y}{d {x}^{n}} + {A}_{n - 1} \frac{{d}^{n - 1} y}{d {x}^{n - 1}} + … + {A}_{1} \frac{d y}{d x} + {A}_{0} y = f ( x ) {A}_{n} , {A}_{n - 1} , … , {A}_{1} , {A}_{0}$$

are constants, and may be solved using known methods if the initial conditions are known.

The n^{th} order linear nonhomogeneous equation may be transformed into the Laplace domain:

$${A}_{n} [ {s}^{n} Y ( s ) - {\sum }_{k = 0}^{n - 1} {s}^{n - 1 - k} {Y}^{( k )} ( 0 ) ] + {A}_{n - 1} [ {s}^{n - 1} Y ( s ) - {\sum }_{k = 0}^{n - 2} {s}^{n - 2 - k} {Y}^{( k )} ( 0 ) ] + …$$

$${A}_{2} [ {s}^{2} Y ( s ) - sY ( 0 ) - {Y}^{′} ( 0 ) ] + {A}_{1} [ sY ( s ) - Y ( 0 ) ] + {A}_{0} Y ( s ) = F ( s ) ,$$

which may then be solved as an algebraic equation in the s-domain for Y(s), which may then be transformed back to the time domain to obtain the solution y(t).

2^{nd} order linear equations may be expressed in the form: y″+p(x)y′+q(x)y=0 or

$$\frac{{d}^{2} y}{{dx}^{2}} + p ( x ) \frac{dy}{dx} + q ( x ) y = 0 ,$$

with linearly independent solutions y₁=y₁(x) and y₂=y₂(x). Then, y(x)=c₁y₁(x)+c₂y₂(x) is the general solution in the sense that every solution can be obtained by choosing appropriate values for the arbitrary constants c₁ and c₂.

If the coefficients are constants, i.e.,

$$\frac{{d}^{2} y}{{dx}^{2}} + p \frac{dy}{dx} + qy = 0$$

where p and q are constants, or

$$a \frac{{d}^{2} y}{{dx}^{2}} + b \frac{dy}{dx} + cy = 0$$

where a, b, and c are constants; replacing d²y/dx² with m², dy/dx with m′, and y with m⁰ will result in am²+bm+c=0, which is called the “auxiliary quadratic equation”. The general solution of the 2^{nd}—order linear differential equation depends on the roots of the auxiliary quadratic equation am²+bm+c=0 such that if b²−4ac>0 (2 distinct real roots m₁ and m₂), then y=c₁e^{m}^{₁}^{x}+c₂e^{m}^{₂}^{x}. If b²−4ac=0 (1 real repeated root m₁=m₂(=m)), then y=c₁e^{mx}+c₂xe^{mx}. If b²−4ac<0 (2 complex roots m₁=α+βi and m₂=α−βi), then y=e^{ax}(c₁ cos βx+c₂ sin βx). Considering

$$\frac{{d}^{2} y}{{dx}^{2}} + p \frac{dy}{dx} + qy = r ( x ) ,$$

where p and q are constants and r(x) is a continuous function, the general solution of y″+py′+qy=r(x) is y(x)=c₁y₁(x)+c₂y₂(x)+yₚ(x), where c₁y₁(x)+c₂y₂(x) is the general solution of the homogeneous equation y″+py′+qy=0, and yₚ(x) is any solution of y″+py′+qy=r(x).

Thus, assuming that the homeostatic mechanisms have governing physics that correspond to differential equations, a rich body of analysis is available to determine the structure of the equation, and the parameters of the equations, such as by measurement of physical states. The techniques include continuous and discrete-time, analog and digital amplitude, and various transform domains of analysis.

### Determination of System Parameters

The step response can be used to measure several key parameters for a physical system. First, any pure delays in the system time shift the output relative to the input. For first-order systems, the time constant can be calculated. For second-order systems, we can measure the damped natural frequency od and damping ratio ζ as follows. First, measure the amplitude and time to peak of the first one or two consecutive cycles. The reduction from the initial cycle amplitude Xₒ to the last cycle amplitude Xₙ for the n cycles is measured.

The following relationship is associated with the logarithmic decrement for under-damped second-order systems:

$$\frac{ζ}{\sqrt{1 - {ζ}^{2}}} = \frac{1}{2 π n} ln ( \frac{{X}_{o}}{{X}_{n}} )$$

For small ζ, this becomes

$$ζ = \frac{1}{2 π n} ln ( \frac{{X}_{o}}{{X}_{n}} )$$

ζ may be solved by first estimating and/or trial and error. Divide the number of cycles, n, by the time taken to complete them (tₙ−tₒ). Convert the resulting frequency in Hz to radians/sec. This damped frequency, ω_{d}, is related to the natural frequency, ωₙ, according to:

$${ω}_{n} = \frac{{ω}_{d}}{\sqrt{1 - {ζ}^{2}}} ,$$

which becomes ωₙ=ω_{d} for lightly damped systems.

The frequency response of a linear system is the output response to sinusoidal inputs. The output response is at the same frequency as the input with differing magnitude and phase. The frequency response characteristics of linear systems may be understood using a Bode plot of magnitude (M_{dB}=20 log₁₀M) and phase (deg) versus log frequency. Typically, the gain is fairly constant up to the corner frequency ƒ_{c}. Above the corner frequency, the amplitude rolls off with a slope of −20 dB/decade. The resonant peak at ƒ=ƒₙ gets larger with smaller damping ratio. The gain is constant below the resonant frequency and rolls off with a slope of −40 dB/decade above the resonant frequency. The frequency response of a second-order system without stiffness and damping (pure inertia) has a gain, which rolls off with a constant −40 dB/dec, and the phase is constant at 180 deg. τ, damping ratios δ, and undamped natural frequencies ωₙ can be determined from experimental measurements.

### Statistical Models

As stated above, discrete physiological data obtained by sampling the physiological parameter represents a time series. Models for time series data can have many forms and represent different stochastic processes. Three broad classes of modelling are the autoregressive (AR) models, the integrated (I) models, and the moving average (MA) models. These three classes depend linearly on previous data points. Combinations of these approaches produce autoregressive moving average (ARMA) and autoregressive integrated moving average (ARIMA) models. The autoregressive fractionally integrated moving average (ARFIMA) model generalizes the three models: AR, L, and MA. Extensions of these classes to deal with vector-valued data are known as multivariate time-series models, and sometimes the preceding acronyms are extended by including an initial “V” for “vector,” as in VAR for vector autoregression. An additional set of extensions of these models is available for use where the observed time-series is driven by some “forcing” time-series (which may not have a causal effect on the observed series): the distinction from the multivariate case is that the forcing series may be deterministic or under the experimenter's control. For these models, the acronyms are extended with a final “X” for “exogenous.”

Non-linear dependence of the level of a series on previous data points is of interest, partly because of the possibility of producing a chaotic time series. However, more importantly. empirical investigations can indicate the advantage of using predictions derived from non-linear models, over those from linear models, as in nonlinear autoregressive exogenous models.

Among other types of non-linear time series models, there are models to represent the changes of variance over time (heteroskedasticity). These models represent autoregressive conditional heteroskedasticity (ARCH), and the collection comprises a wide variety of representation (GARCH, TARCH, EGARCH, FIGARCH, CGARCH, etc.). Here changes in variability are related to, or predicted by, recent past values of the observed series. This is in contrast to other possible representations of locally varying variability, where the variability might be modelled as being driven by a separate time-varying process, as in a doubly stochastic model.

In recent work on model-free analyses, wavelet-transform-based methods (for example, locally stationary wavelets and wavelet decomposed neural networks) have gained favor. Multiscale (often referred to as multiresolution) techniques decompose a given time series, attempting to illustrate time dependence at multiple scales, Markov switching multifractal (MSMF) techniques are used for modelling volatility evolution.

A Hidden Markov model (HMM) is a statistical Markov model in which the system being modelled is assumed to be a Markov process with unobserved (hidden) states. An HMM can be considered as the simplest dynamic Bayesian network.

Tools for investigating time-series data include: consideration of the autocorrelation function and the spectral density function (also cross-correlation functions and cross-spectral density functions); scaled cross- and auto-correlation functions to remove contributions of slow components; performing a Fourier transform to investigate the series in the frequency domain; use of a filter to remove unwanted noise; Principal Component Analysis (PCI), Independent Component Analysis (ICA), or empirical orthogonal function analysis, singular spectrum analysis; “structural” models: General State Space Models, Unobserved Components Models; Machine Learning: Artificial neural networks, Support vector machine, Fuzzy logic, Gaussian process, Hidden Markov model; Queueing theory analysis; Control chart, Shewhart individuals control chart, CUSUM chart, EWMA chart; Detrended fluctuation analysis; Dynamic time warping; Cross-correlation; Dynamic Bayesian network; Time-frequency analysis techniques: Fast Fourier transform, Continuous wavelet transform, Short-time Fourier transform, Chirplet transform, Fractional Fourier transform; Chaotic analysis: Correlation dimension, Recurrence plots, Recurrence quantification analysis, Lyapunov exponents, Entropy encoding. See en.wikipedia.org/wiki/Time_series #Models.

### Nyquist Stability Criterion

The Nyquist stability criterion is useful because it allows us to gain insight into the stability of a closed-loop plant based on properties of an open-loop plant. Consider a simple closed-loop feedback system with forward gain G(s) and feedback gain of 1, u is a reference command, y is the output. It may be readily shown that the closed-loop transfer function H(s) may be expressed

$$\frac{y}{u} = H ( s ) = \frac{G ( s )}{1 + G ( s )} .$$

It is convenient to break G(s) into a numerator and denominator

$$G ( s ) = \frac{n ( s )}{d ( s )}$$

whereupon H(s) may be expressed

$$H ( s ) = \frac{n ( s )}{n ( s ) + d ( s )} .$$

It is also readily shown that

$$1 + G ( s ) = \frac{d ( s ) + n ( s )}{d ( s )} .$$

Let us define N_{cwe} as the number of clockwise (CW) encirclements of the origin by the Nyquist contour of 1+G(s). N_{cwe}=N_{z}−Nₚ. Since we are dealing with 1+G(s), the number of zeros encircled will be the number of zeros of n(s)+d(s) in the right half-plane, or in other words, the number of zeros is the number of unstable closed-loop poles, which we will denote N_{uclp}. Thus N_{z}=N_{uclp}. The number of poles circled by the Nyquist contour is the number of zeros of d(s) enclosed by the Nyquist contour, but these are the unstable poles of the open-loop transfer function. Thus Nₚ=Nᵤₒₗₚ, where Nᵤₒₗₚ denotes the number of unstable open-loop poles. The number of unstable closed-loop poles may be expressed N_{uclp}=N_{cwe}+Nᵤₒₗₚ. Since N_{cwe} is the number of clockwise encirclements of the origin by the Nyquist evaluation of 1+G(s), and 1+G(s) is just G(s) shifted by 1, N_{cwe} is also equal to the number of clockwise encirclements of −1 by the Nyquist evaluation of G(s). This leads to the Nyquist Immittance Criterion: The number of unstable closed-loop poles is equal to the number of unstable open-loop poles plus the number of clockwise encirclements of −1 by the contour evaluation of G(s) over the Nyquist contour.

Thus, suppose we have knowledge of the open-loop transfer function G(s), and we know the number of unstable open-loop poles Nᵤₒₗₚ. Then, we can perform a Nyquist evaluation of G(s) and count the number of clockwise encirclements. Thus, we may use Nyquist theory to predict the stability of the closed-loop system based on properties of the open-loop plant G(s). The path for the Nyquist contour starts at s=−jωₘₐₓ, proceeds upward along the jωₘₐₓ axis through s=0 until s=jωₘₐₓ and then proceed in a circular arc of radius |s|=ωₘₐₓ through s=ωₘₐₓ, and then back to s=−jωₘₐₓ. This path is denoted P_{N}. By allowing ωₘₐₓ→∞, this path encompasses the entire right half plane. The Nyquist Immittance Criterion provides at a source-load system is stable provided that the Nyquist evaluation of ZₛYₗ does not encircle −1, where Zₛ is the source impedance, and Yₗ is the load admittance. As a test for stability, the Nyquist Immittance Criterion can be directly applied. Various stability criteria all have interpretations in the s-plane, and in each case, each Stability Criterion forms a boundary in the s-plane; if the Nyquist evaluation of ZₛYₗ does not cross this boundary, then −1 cannot be encircled and therefore the system must be stable. The Middlebrook Criterion consists of a circle of radius 1/GM in the s-plane where GM denotes the gain margin. If the Nyquist plot of ZₛYₗ is always within the circle, then encirclements of the −1 point cannot occur, provided the gain margin is greater than 1. For a given Zₛ, the range of allowable Yₗ is readily established; in particular

$$\vert {Y}_{l} \vert < \frac{1}{\vert {Z}_{z} \vert GM} .$$

For a frequency s=jω, it is convenient to denote the real and imaginary part of N(jω) as Nᵣ and Nᵢ, respectively, whereupon it can be shown that the upper half of the Nyquist contour may be expressed as

$$\vert {N}_{i} \vert = \vert rC - L / R \vert {( \frac{- {N}_{r} - r / R}{LC} )}^{1 / 2} .$$

It is also convenient to designate an arbitrary stability criterion by a parameterized curve in the s-plane given by Mᵣ(x)+jMᵢ(x) where x is an independent variable. It is apparent that the slope d|Nᵢ|/d(−Nᵣ)→0 as −Nᵣ→∞ for all values of parameter r, C, L, and R. As a result, the Nyquist contour will violate any stability criterion which has a non-zero slope d|Mᵢ|/d(−Mᵣ) as −Mᵣ→∞.

Various types of devices to measure physiological parameters are known, especially wearable and implantable devices. U.S. Pat. Nos. 6,453,195; 6,595,929; 6,675,049; 6,817,979; 7,020,508; 7,044,911; 7,108,659; 7,167,743; 7,261,690; 7,273,457; 7,285,090; 7,291,114; 7,313,440; 7,330,760; 7,366,572; 7,395,113; 7,407,484; 7,447,545; 7,463,142; 7,491,181; 7,542,803; 7,561,919; 7,577,475; 7,590,453; 7,590,455; 7,598,878; 7,610,094; 7,616,991; 7,623,919; 7,630,755; 7,641,619; 7,695,512; 7,697,994; 7,717,848; 7,761,261; 7,765,012; 7,775,993; 7,782,192; 7,787,946; 7,792,583; 7,801,591; 7,805,196; 7,819,909; 7,825,794; 7,848,819; 7,881,798; 7,885,712; 7,904,149; 7,908,000; 7,908,013; 7,953,488; 7,959,567; 7,970,470; 7,978,062; 8,005,539; 8,010,189; 8,021,299; 8,032,224; 8,033,999; 8,038,613; 8,055,330; 8,055,334; 8,055,348; 8,078,278; 8,086,250; 8,086,302; 8,114,021; 8,115,618; 8,121,691; 8,121,694; 8,135,473; 8,137,269; 8,140,161; 8,152,710; 8,157,731; 8,160,695; 8,160,702; 8,165,691; 8,165,893; 8,172,459; 8,174,378; 8,174,395; 8,180,446; 8,180,447; 8,185,181; 8,190,253; 8,204,590; 8,204,597; 8,214,045; 8,217,946; 8,233,976; 8,246,563; 8,249,708; 8,251,061; 8,251,903; 8,260,412; 8,260,422; 8,265,723; 8,265,771; 8,270,938; 8,271,072; 8,273,032; 8,287,520; 8,290,596; 8,295,933; 8,301,219; 8,306,610; 8,308,661; 8,310,336; 8,321,003; 8,328,420; 8,332,038; 8,335,568; 8,337,431; 8,355,783; 8,359,095; 8,373,556; 8,376,943; 8,395,498; 8,396,554; 8,398,546; 8,406,893; 8,410,940; 8,412,352; 8,417,311; 8,419,650; 8,428,744; 8,435,186; 8,435,738; 8,436,810; 8,438,038; 8,447,265; 8,447,401; 8,447,403; 8,447,404; 8,452,366; 8,452,394; 8,457,727; 8,460,189; 8,475,739; 8,485,979; 8,493,187; 8,509,893; 8,512,242; 8,515,547; 8,515,559; 8,521,292; 8,527,045; 8,527,064; 8,532,779; 8,540,632; 8,545,436; 8,547,248; 8,554,325; 8,554,331; 8,565,886; 8,571,881; 8,583,227; 8,587,427; 8,606,355; 8,611,996; 8,615,299; 8,615,377; 8,615,405; 8,620,591; 8,620,617; 8,620,679; 8,630,706; 8,632,463; 8,641,612; 8,655,441; 8,663,106; 8,666,488; 8,674,825; 8,676,170; 8,679,014; 8,688,210; 8,688,221; 8,694,282; 8,698,638; 8,706,232; 8,706,233; 8,706,237; 8,708,904; 8,712,529; 8,712,530; 8,715,269; 8,718,193; 8,725,244; 8,725,253; 8,730,031; 8,738,323; 8,742,623; 8,744,587; 8,744,803; 8,744,804; 8,751,194; 8,755,871; 8,758,242; 8,761,852; 8,762,101; 8,762,102; 8,766,789; 8,766,805; 8,768,648; 8,774,918; 8,775,120; 8,781,791; 8,788,028; 8,792,982; 8,792,991; 8,793,101; 8,805,508; 8,805,518; 8,805,646; 8,812,259; 8,812,260; 8,818,505; 8,818,522; 8,818,753; 8,821,350; 8,827,906; 8,834,020; 8,838,235; 8,840,838; 8,847,766; 8,849,610; 8,849,682; 8,852,095; 8,852,098; 8,858,432; 8,870,736; 8,870,742; 8,870,766; 8,879,983; 8,880,155; 8,882,666; 8,884,809; 8,892,401; 8,907,782; 8,920,332; 8,923,970; 8,923,976; 8,929,963; 8,932,221; 8,935,119; 8,935,123; 8,938,368; 8,942,953; 8,945,017; 8,945,328; 8,948,832; 8,948,839; 8,954,149; 8,954,290; 8,954,291; 8,956,287; 8,956,288; 8,956,303; 8,957,777; 8,958,870; 8,961,412; 8,961,414; 8,965,730; 8,965,824; 8,968,196; 8,970,392; 8,971,936; 8,979,757; 8,979,763; 8,989,858; 8,989,861; 8,990,924; 8,998,815; 9,000,973; 9,005,129; 9,014,790; 9,014,802; 9,019,106; 9,020,591; 9,020,592; 9,020,597; 9,026,190; 9,026,201; 9,026,206; 9,031,669; 9,031,812; 9,033,875; 9,033,876; 9,033,920; 9,039,614; 9,042,971; 9,044,149; 9,044,150; 9,044,171; 9,044,180; 9,049,998; 9,050,041; 9,060,746; 9,061,147; 9,064,342; 9,066,209; 9,072,447; 9,072,870; 9,077,030; 9,079,045; 9,079,060; 9,081,534; 9,083,589; 9,101,334; 9,103,899; 9,108,098; 9,113,794; 9,113,795; 9,119,554; 9,128,015; 9,138,181; 9,138,523; 9,138,537; 9,142,117; 9,148,483; 9,149,577; 9,151,834; 9,155,885; 9,164,167; 9,165,117; 9,168,001; 9,168,374; 9,168,419; 9,171,201; 9,174,058; 9,185,489; 9,186,509; 9,192,329; 9,198,604; 9,204,806; 9,205,264; 9,215,290; 9,218,454; 9,220,461; 9,226,663; 9,227,128; 9,233,245; 9,237,855; 9,237,858; 9,241,635; 9,247,911; 9,250,229; 9,254,092; 9,259,180; 9,265,871; 9,268,915; 9,269,251; 9,270,025; 9,277,534; 9,282,894; 9,282,902; 9,288,298; 9,288,614; 9,289,123; 9,289,607; 9,289,613; 9,295,403; 9,302,045; 9,307,917; 9,310,909; 9,317,729; 9,320,677; 9,320,842; 9,323,894; 9,326,708; 9,326,711; 9,332,919; 9,333,350; 9,339,201; 9,339,203; 9,344,546; 9,345,879; 9,345,892; 9,348,974; 9,352,156; 9,358,374; 9,369,365; 9,370,320; 9,370,326; 9,374,279; 9,375,145; 9,386,924; 9,390,427; 9,393,384; 9,398,856; 9,402,545; 9,402,552; 9,410,979; 9,415,125; 9,418,390; 9,420,083; 9,420,956; 9,427,165; 9,427,581; 9,431,694; 9,433,371; 9,433,783; 9,439,567; 9,439,599; 9,444,503; 9,445,767; 9,446,235; 9,454,644; 9,456,755; 9,456,787; 9,462,979; 9,463,325; 9,480,424; 9,486,128; 9,492,084; 9,492,657; 9,498,137; 9,498,624; 9,504,408; 9,504,425; 9,506,802; 9,510,788; 9,520,638; 9,521,868; 9,521,962; 9,522,317; 9,526,433; 9,526,859; 9,529,972; 9,532,722; 9,533,157; 9,538,921; 9,538,922; 9,543,636; 9,554,705; 9,554,719; 9,559,353; 9,561,367; 9,568,492; 9,572,499; 9,572,533; 9,572,992; 9,578,903; 9,579,020; 9,579,048; 9,579,060; 9,579,516; 9,583,256; 9,585,566; 9,585,606; 9,592,328; 9,592,379; 9,592,508; 9,595,996; 9,596,997; 9,597,014; 9,597,023; 9,597,505; 9,599,632; 9,600,676; 9,603,569; 9,613,184; 9,614,337; 9,615,215; 9,615,794; 9,616,225; 9,622,691; 9,623,238; 9,623,248; 9,632,102; 9,633,170; 9,639,170; 9,641,239; 9,641,469; 9,645,143; 9,646,481; 9,651,533; 9,655,519; 9,655,548; 9,658,066; 9,659,484; 9,662,053; 9,664,556; 9,664,702; 9,669,262; 9,672,393; 9,672,715; 9,672,754; 9,680,831; 9,681,814; 9,681,842; 9,685,802; 9,687,658; 9,692,230; 9,692,844; 9,693,696; 9,696,199; 9,700,222; 9,700,223; 9,706,957; 9,707,466; 9,712,629; 9,713,445; 9,713,701; 9,719,990; 9,723,986; 9,724,509; 9,724,510; 9,724,511; 9,724,517; 9,724,521; 9,724,562; 9,730,025; 9,730,601; 9,730,619; 9,730,625; 9,734,304; 9,736,603; 9,747,431; 9,749,232; 9,750,977; 9,752,925; 9,755,704; 9,756,169; 9,757,040; 9,763,616; 9,766,959; 9,769,564; 9,770,185; 9,775,987; 9,776,042; 9,778,280; 9,782,084; 9,782,132; 9,788,785; 9,789,252; 9,789,309; 9,789,315; 9,795,323; 9,795,782; 9,801,547; 9,808,198; 9,808,204; 9,810,704; 9,814,388; 9,814,400; 9,814,886; 9,817,440; 9,820,698; 9,826,903; 9,826,940; 9,830,781; 9,833,254; 9,847,012; 9,848,789; 9,854,986; 9,855,433; 9,861,286; 9,864,842; 9,868,332; 9,869,973; 9,872,968; 9,876,537; 9,877,650; 9,882,610; 9,883,800; 9,885,698; 9,888,848; 9,889,305; 9,894,691; 9,895,063; 9,900,287; 9,901,269; 9,901,305; 9,901,741; 9,905,105; 9,913,591; 9,913,617; 9,913,619; 9,921,726; 9,931,040; 9,936,916; 9,936,919; 9,943,269; 9,943,461; 9,943,719; 9,950,236; 9,952,095; 9,952,240; 9,953,041; 9,955,919; 9,956,393; 9,956,470; 9,965,059; 9,968,788; 9,974,484; 9,977,578; 9,985,825; 9,986,771; 9,987,489; 9,987,497; 9,991,920; 9,993,207; 9,998,804; 9,999,355; RE44408; RE45766; U.S. Pat. Nos. 10,003,862; 10,004,406; 10,004,949; 10,007,758; 10,008,090; 10,010,278; 10,010,753; 10,012,664; 10,016,613; 10,018,643; 10,021,733; 10,022,061; 10,022,062; 10,028,037; 10,028,659; 10,028,660; 10,029,172; 10,032,002; 10,034,625; 10,039,928; 10,043,354; 10,045,117; 10,045,439; 10,052,486; 10,054,909; 10,055,550; 10,058,274; 10,060,788; 10,064,562; 10,070,805; 10,071,285; 10,076,282; 10,076,462; 10,080,499; 10,080,527; 10,080,530; 10,085,643; 10,088,356; 10,092,193; 10,092,203; 10,092,355; 10,092,692; 10,095,649; 10,095,837; 10,098,546; 10,098,548; 10,098,558; 10,098,810; 10,099,053; 10,104,026; 10,105,080; 10,105,100; 10,105,487; 10,105,547; 10,108,785; 10,109,175; 10,117,600; 10,117,606; 10,118,035; 10,118,041; 10,119,715; 10,122,421; 10,124,179; 10,126,283; 10,126,998; 10,130,305; 10,130,550; 10,132,677; 10,135,076; 10,136,817; 10,136,819; 10,137,230; 10,140,842; 10,141,073; 10,143,395; 10,143,840; 10,143,847; 10,149,616; 10,152,957; 10,153,537; 10,154,129; 10,154,922; 10,155,110; 10,159,415; 10,163,314; 10,165,977; 10,166,164; 10,166,333; 10,172,523; 10,176,412; 10,178,973; 10,179,064; 10,179,246; 10,182,336; 10,182,729; 10,188,296; 10,188,299; 10,194,418; 10,194,702; 10,194,808; 10,194,809; 10,194,816; 10,194,836; 10,201,283; 10,201,295; 10,201,702; 10,207,041; 10,209,365; 10,210,741; 10,213,113; 10,213,150; 10,215,619; 10,218,433; 10,219,746; 10,219,754; 10,223,459; 10,226,187; 10,226,188; 10,226,217; 10,226,396; 10,227,063; 10,230,699; 10,231,666; 10,231,784; 10,234,330; 10,248,302; 10,251,571; 10,251,595; 10,252,140; 10,255,994; 10,258,092; 10,258,288; 10,258,828; 10,264,971; 10,269,452; 10,271,587; 10,271,792; 10,272,242; 10,278,217; 10,278,624; 10,279,152; 10,279,200; 10,279,201; 10,284,537; 10,289,806; 10,292,611; 10,292,631; 10,293,158; 10,293,208; 10,299,693; 10,299,725; 10,299,734; 10,299,736; 10,300,283; 10,302,469; 10,305,692; 10,307,101; 10,307,111; 10,311,696; 10,311,706; 10,314,488; 10,314,546; 10,314,547; 10,325,681; 10,327,674; 10,327,689; 10,327,984; 10,328,228; 10,328,266; 20020019586; 20020133196; 20020177782; 20020183646; 20030004403; 20030018369; 20030107487; 20030220579; 20040002634; 20040034289; 20040039254; 20040078067; 20040133081; 20040147818; 20040152956; 20040152957; 20040158194; 20040176822; 20040186390; 20050060030; 20050061323; 20050065572; 20050075213; 20050076909; 20050080322; 20050101845; 20050115561; 20050116820; 20050136385; 20050142070; 20050148828; 20050165456; 20050182389; 20050209511; 20050209512; 20050209513; 20050209643; 20050209644; 20050209645; 20050215847; 20050215947; 20050216064; 20050222522; 20050222643; 20050234514; 20050234518; 20050245988; 20060020297; 20060030890; 20060064030; 20060085040; 20060122474; 20060122864; 20060149324; 20060149330; 20060149331; 20060224051; 20060235472; 20060253005; 20060264730; 20060265024; 20060265025; 20060287677; 20060293571; 20070015976; 20070021979; 20070060800; 20070088521; 20070100666; 20070106138; 20070106172; 20070123758; 20070162090; 20070173705; 20070239054; 20070239230; 20070249968; 20070250121; 20070250134; 20070255118; 20070260491; 20070265508; 20070276439; 20070293737; 20080015421; 20080051667; 20080071150; 20080071324; 20080071326; 20080071327; 20080077440; 20080091092; 20080103534; 20080129486; 20080131362; 20080132981; 20080132982; 20080140161; 20080140162; 20080140163; 20080161654; 20080161655; 20080167535; 20080167536; 20080167537; 20080167538; 20080167539; 20080167700; 20080171919; 20080171920; 20080171921; 20080171922; 20080172102; 20080177355; 20080195249; 20080214903; 20080249806; 20080250340; 20080250341; 20080275309; 20080275349; 20080287751; 20080287817; 20080294020; 20080294024; 20080300449; 20080300470; 20080300649; 20080300650; 20080300651; 20080306357; 20080312511; 20090030263; 20090036951; 20090040041; 20090058635; 20090058636; 20090062887; 20090063193; 20090076343; 20090076346; 20090082640; 20090082641; 20090083070; 20090099627; 20090105605; 20090105785; 20090112071; 20090113295; 20090118599; 20090128487; 20090131739; 20090138207; 20090149797; 20090149798; 20090149799; 20090149895; 20090149896; 20090149897; 20090149914; 20090156309; 20090171163; 20090177068; 20090192556; 20090222065; 20090228078; 20090234916; 20090259216; 20090264789; 20090264955; 20090264956; 20090264957; 20090264967; 20090270942; 20090276002; 20090276004; 20090281594; 20090292180; 20090299428; 20090306740; 20090312622; 20090326346; 20090326350; 20090326356; 20100016918; 20100022856; 20100022861; 20100030043; 20100030088; 20100030090; 20100030286; 20100030289; 20100030293; 20100036211; 20100041975; 20100049010; 20100058462; 20100069841; 20100106212; 20100114195; 20100114196; 20100114197; 20100114198; 20100114199; 20100114200; 20100114201; 20100114202; 20100114203; 20100114204; 20100114208; 20100114209; 20100114216; 20100114217; 20100114221; 20100114224; 20100114237; 20100114241; 20100114244; 20100121170; 20100121215; 20100121413; 20100152815; 20100160014; 20100160800; 20100160804; 20100168607; 20100174155; 20100174180; 20100185064; 20100185225; 20100194631; 20100198034; 20100198279; 20100198280; 20100198284; 20100198291; 20100198308; 20100210924; 20100222845; 20100222846; 20100228314; 20100238019; 20100245077; 20100249540; 20100268040; 20100273738; 20100274100; 20100274106; 20100280330; 20100280334; 20100280335; 20100280336; 20100280574; 20100280579; 20100305665; 20100308974; 20100311388; 20110003664; 20110022123; 20110040197; 20110040546; 20110040547; 20110046697; 20110054270; 20110061647; 20110076984; 20110077706; 20110082377; 20110093040; 20110093046; 20110105860; 20110105873; 20110105921; 20110106200; 20110112442; 20110118805; 20110130092; 20110144463; 20110144967; 20110152632; 20110160623; 20110160796; 20110172504; 20110172545; 20110179637; 20110183305; 20110184482; 20110184483; 20110190570; 20110190580; 20110190581; 20110190595; 20110190654; 20110208012; 20110208015; 20110237916; 20110245633; 20110264034; 20110273287; 20110295335; 20110295336; 20110301441; 20110301662; 20110307027; 20110307028; 20120001751; 20120001920; 20120003933; 20120010543; 20120022340; 20120029586; 20120059389; 20120083650; 20120094649; 20120095306; 20120108998; 20120109237; 20120116475; 20120123232; 20120130286; 20120130444; 20120132211; 20120136261; 20120136413; 20120149996; 20120150258; 20120172652; 20120197336; 20120197337; 20120197338; 20120197349; 20120197350; 20120203118; 20120220986; 20120245439; 20120249324; 20120265026; 20120265031; 20120273354; 20120277546; 20120283577; 20120283578; 20120296184; 20120321759; 20120330112; 20130006076; 20130018239; 20130018668; 20130030259; 20130035575; 20130035740; 20130035865; 20130035871; 20130053657; 20130053913; 20130060098; 20130072998; 20130074614; 20130078149; 20130078624; 20130078625; 20130078733; 20130079236; 20130079599; 20130079646; 20130079840; 20130082837; 20130085401; 20130085679; 20130102859; 20130104288; 20130109997; 20130124039; 20130135108; 20130147622; 20130154838; 20130154851; 20130158372; 20130165819; 20130172759; 20130191513; 20130217979; 20130218070; 20130218232; 20130225968; 20130229287; 20130245480; 20130253334; 20130253351; 20130253380; 20130274584; 20130274705; 20130289446; 20130289659; 20130289664; 20130294969; 20130296669; 20130296670; 20130297330; 20130297344; 20130303843; 20130310706; 20130310896; 20130317753; 20130325394; 20130325396; 20130325404; 20130331660; 20130331919; 20130333054; 20130338448; 20140012105; 20140031787; 20140035761; 20140036643; 20140039804; 20140039839; 20140039840; 20140039841; 20140039842; 20140043149; 20140046391; 20140049377; 20140051962; 20140052790; 20140056757; 20140062718; 20140066844; 20140067423; 20140073043; 20140073969; 20140077956; 20140081100; 20140081665; 20140081667; 20140088664; 20140088922; 20140089399; 20140089514; 20140095420; 20140100432; 20140107433; 20140107509; 20140107511; 20140107513; 20140107567; 20140135594; 20140135631; 20140139405; 20140142403; 20140142549; 20140145915; 20140156043; 20140156228; 20140163432; 20140163483; 20140163647; 20140163927; 20140164320; 20140164611; 20140170735; 20140171749; 20140172310; 20140172362; 20140176475; 20140180020; 20140180021; 20140180022; 20140180023; 20140180137; 20140180358; 20140186238; 20140188516; 20140191866; 20140191867; 20140194701; 20140200421; 20140203797; 20140206955; 20140206959; 20140206976; 20140213855; 20140213856; 20140213857; 20140213926; 20140213941; 20140221769; 20140221770; 20140221774; 20140221784; 20140221785; 20140221787; 20140221788; 20140221789; 20140221790; 20140221791; 20140221855; 20140222101; 20140222106; 20140222174; 20140223406; 20140223407; 20140223421; 20140228649; 20140228911; 20140232516; 20140234949; 20140236493; 20140236538; 20140237028; 20140240122; 20140245161; 20140249594; 20140249600; 20140249605; 20140249774; 20140257055; 20140257058; 20140257540; 20140258220; 20140266939; 20140273858; 20140275812; 20140275813; 20140275850; 20140275852; 20140275854; 20140276119; 20140276130; 20140276192; 20140277250; 20140278220; 20140278229; 20140288390; 20140288391; 20140288392; 20140288435; 20140288436; 20140288438; 20140296089; 20140296658; 20140297217; 20140297218; 20140305204; 20140306807; 20140307878; 20140308661; 20140309505; 20140316191; 20140316192; 20140316305; 20140318699; 20140320284; 20140324118; 20140330094; 20140336980; 20140337450; 20140337451; 20140340221; 20140342328; 20140343370; 20140343892; 20140350636; 20140358012; 20140358024; 20140361147; 20140364705; 20140371556; 20140374276; 20140378787; 20150005650; 20150005652; 20150005911; 20150018660; 20150018702; 20150019135; 20150019257; 20150025393; 20150025394; 20150031964; 20150031970; 20150039040; 20150039053; 20150040282; 20150045634; 20150050888; 20150057718; 20150065786; 20150065826; 20150073723; 20150076909; 20150080746; 20150088457; 20150094552; 20150094914; 20150097701; 20150099943; 20150100270; 20150102923; 20150112151; 20150112170; 20150113417; 20150119728; 20150119951; 20150119952; 20150122018; 20150125945; 20150126822; 20150134107; 20150134345; 20150134346; 20150137997; 20150141769; 20150141873; 20150142074; 20150142082; 20150143601; 20150148637; 20150148697; 20150148868; 20150149096; 20150149217; 20150150453; 20150150505; 20150154364; 20150156749; 20150157256; 20150157269; 20150157512; 20150164321; 20150164322; 20150164349; 20150164376; 20150170504; 20150173628; 20150173631; 20150173674; 20150174406; 20150190053; 20150193612; 20150196256; 20150201853; 20150201854; 20150206408; 20150207915; 20150208943; 20150223700; 20150223708; 20150224310; 20150230722; 20150230735; 20150230761; 20150238097; 20150243967; 20150245797; 20150255858; 20150258261; 20150258415; 20150265150; 20150265164; 20150265195; 20150265207; 20150265214; 20150265217; 20150265903; 20150269009; 20150272511; 20150278453; 20150282711; 20150282713; 20150282767; 20150283365; 20150283386; 20150288772; 20150289797; 20150289798; 20150289799; 20150289800; 20150289808; 20150289809; 20150289810; 20150289811; 20150289812; 20150289820; 20150294574; 20150294575; 20150294576; 20150294583; 20150294594; 20150297103; 20150297134; 20150297904; 20150305682; 20150314166; 20150317515; 20150320588; 20150321084; 20150331997; 20150335385; 20150335507; 20150338428; 20150339946; 20150347689; 20150351695; 20150351698; 20150359429; 20150359489; 20150359490; 20150364018; 20150364938; 20150366746; 20150368717; 20150371516; 20150374289; 20160000385; 20160000640; 20160000642; 20160000984; 20160001034; 20160001071; 20160003823; 20160005299; 20160007925; 20160008632; 20160011215; 20160011225; 20160015280; 20160015972; 20160022193; 20160025760; 20160025763; 20160029905; 20160030741; 20160032361; 20160033544; 20160034696; 20160036118; 20160038037; 20160038038; 20160038042; 20160038043; 20160038055; 20160038744; 20160047787; 20160051169; 20160054343; 20160058286; 20160058331; 20160058375; 20160059010; 20160059064; 20160066803; 20160066844; 20160069919; 20160069920; 20160069921; 20160072690; 20160073884; 20160073914; 20160074278; 20160074600; 20160077015; 20160078061; 20160080166; 20160081574; 20160081622; 20160082187; 20160084863; 20160084869; 20160089028; 20160089575; 20160095550; 20160103123; 20160117951; 20160124009; 20160135516; 20160135695; 20160135696; 20160135697; 20160135741; 20160135742; 20160135743; 20160136882; 20160139156; 20160143548; 20160143584; 20160147964; 20160148531; 20160151603; 20160158426; 20160158552; 20160169880; 20160169923; 20160174840; 20160174898; 20160183818; 20160193462; 20160198961; 20160205450; 20160206215; 20160206922; 20160213314; 20160213334; 20160216286; 20160216287; 20160224130; 20160231402; 20160232131; 20160232137; 20160232201; 20160232244; 20160232807; 20160234143; 20160234184; 20160234595; 20160235374; 20160238439; 20160238440; 20160238441; 20160238443; 20160238444; 20160239624; 20160240721; 20160242646; 20160242654; 20160242665; 20160243373; 20160253471; 20160256095; 20160256097; 20160256106; 20160256350; 20160256697; 20160259426; 20160261458; 20160262670; 20160263382; 20160270656; 20160278638; 20160278669; 20160279021; 20160279022; 20160279023; 20160279024; 20160279025; 20160279435; 20160283706; 20160285985; 20160287879; 20160287887; 20160296114; 20160296116; 20160296145; 20160296169; 20160296759; 20160300028; 20160302706; 20160302707; 20160303371; 20160310022; 20160310048; 20160314670; 20160317744; 20160320381; 20160321400; 20160321598; 20160321599; 20160323401; 20160324472; 20160324478; 20160324488; 20160325143; 20160327476; 20160331257; 20160331273; 20160331986; 20160331987; 20160334124; 20160337843; 20160342744; 20160342762; 20160346501; 20160346530; 20160346542; 20160346609; 20160354606; 20160361026; 20160361027; 20160367803; 20160370396; 20160373161; 20160374600; 20160374608; 20160374618; 20160374620; 20160374625; 20160377640; 20160378069; 20160378070; 20160378071; 20170000324; 20170000325; 20170000326; 20170000329; 20170000330; 20170000331; 20170000332; 20170000333; 20170000334; 20170000335; 20170000337; 20170000340; 20170000341; 20170000342; 20170000343; 20170000345; 20170000371; 20170000372; 20170000375; 20170000390; 20170000391; 20170000415; 20170000454; 20170000683; 20170000936; 20170001032; 20170004106; 20170007111; 20170007115; 20170007116; 20170007122; 20170007123; 20170007182; 20170007450; 20170007799; 20170007843; 20170010469; 20170010470; 20170017083; 20170021171; 20170021172; 20170027514; 20170027515; 20170027523; 20170027812; 20170028231; 20170036021; 20170036031; 20170038401; 20170043160; 20170045862; 20170049352; 20170049406; 20170049946; 20170053078; 20170055205; 20170055845; 20170055851; 20170055882; 20170055887; 20170055896; 20170063434; 20170065183; 20170065821; 20170071474; 20170071487; 20170071506; 20170071545; 20170080207; 20170084983; 20170086519; 20170086752; 20170095011; 20170095205; 20170095673; 20170095721; 20170097994; 20170098367; 20170100056; 20170103669; 20170112391; 20170112439; 20170112671; 20170113042; 20170113057; 20170118626; 20170119312; 20170124853; 20170127975; 20170128140; 20170128722; 20170128735; 20170135633; 20170136209; 20170136264; 20170136265; 20170136842; 20170143219; 20170143233; 20170143266; 20170143267; 20170143268; 20170143282; 20170146385; 20170146386; 20170146387; 20170146388; 20170146389; 20170146390; 20170146391; 20170147754; 20170147837; 20170148240; 20170149773; 20170150895; 20170156662; 20170156663; 20170157411; 20170160398; 20170164850; 20170164876; 20170164878; 20170165483; 20170168457; 20170169190; 20170172522; 20170181708; 20170182330; 20170188893; 20170189756; 20170189757; 20170189815; 20170193140; 20170195475; 20170196455; 20170196457; 20170196458; 20170196513; 20170202461; 20170202484; 20170209053; 20170209055; 20170209081; 20170215729; 20170215756; 20170215757; 20170216125; 20170221463; 20170224252; 20170224260; 20170224268; 20170224581; 20170224990; 20170228516; 20170228627; 20170230084; 20170231494; 20170231495; 20170231597; 20170232297; 20170237694; 20170238814; 20170238881; 20170239470; 20170239523; 20170243056; 20170243508; 20170246459; 20170248567; 20170249115; 20170251232; 20170251940; 20170251974; 20170251975; 20170255262; 20170257162; 20170258389; 20170258585; 20170262015; 20170262604; 20170264338; 20170264693; 20170265769; 20170265770; 20170266533; 20170270765; 20170273629; 20170274267; 20170281020; 20170282011; 20170283845; 20170290513; 20170290528; 20170290980; 20170293266; 20170296052; 20170296076; 20170296107; 20170296139; 20170296813; 20170296814; 20170300653; 20170301214; 20170303784; 20170303786; 20170308663; 20170311878; 20170312161; 20170312165; 20170312612; 20170312746; 20170316182; 20170319119; 20170319122; 20170319123; 20170319849; 20170324437; 20170325056; 20170325524; 20170325525; 20170325727; 20170326013; 20170330447; 20170332980; 20170333752; 20170340920; 20170344736; 20170347895; 20170350878; 20170354351; 20170354365; 20170354547; 20170354795; 20170356770; 20170357217; 20170357419; 20170358239; 20170358240; 20170358242; 20170360320; 20170361092; 20170361162; 20170366233; 20170367576; 20170367585; 20170367599; 20170372009; 20170372026; 20170372216; 20170374436; 20180000336; 20180000345; 20180000347; 20180000414; 20180001005; 20180001023; 20180001083; 20180007467; 20180008151; 20180008191; 20180008193; 20180008206; 20180008831; 20180012469; 20180021564; 20180021589; 20180027347; 20180028106; 20180028114; 20180028122; 20180028809; 20180035898; 20180035920; 20180035982; 20180040258; 20180042526; 20180042809; 20180043173; 20180045745; 20180047074; 20180049251; 20180049675; 20180055373; 20180055382; 20180055386; 20180056071; 20180060449; 20180067516; 20180075716; 20180075724; 20180078754; 20180078777; 20180085011; 20180085021; 20180085572; 20180085576; 20180085580; 20180085585; 20180085586; 20180089394; 20180090229; 20180092551; 20180092554; 20180092577; 20180101138; 20180101655; 20180103863; 20180103874; 20180103883; 20180104407; 20180108440; 20180114124; 20180116536; 20180116723; 20180117346; 20180125689; 20180126172; 20180126222; 20180132032; 20180132778; 20180139799; 20180140191; 20180140198; 20180140835; 20180147024; 20180147333; 20180147449; 20180152972; 20180153404; 20180153475; 20180156660; 20180162186; 20180168460; 20180168461; 20180168463; 20180168905; 20180169411; 20180169412; 20180169421; 20180173405; 20180174686; 20180177397; 20180177459; 20180182489; 20180184735; 20180184901; 20180184914; 20180189452; 20180191693; 20180192953; 20180203882; 20180206747; 20180207429; 20180213583; 20180214025; 20180214026; 20180214066; 20180214080; 20180219334; 20180221645; 20180225419; 20180228434; 20180228438; 20180229674; 20180235537; 20180238734; 20180240176; 20180240357; 20180241564; 20180242864; 20180242921; 20180242926; 20180243528; 20180243541; 20180247712; 20180249919; 20180250574; 20180256076; 20180256096; 20180260530; 20180261066; 20180270549; 20180279901; 20180279952; 20180279965; 20180280177; 20180289166; 20180289275; 20180289310; 20180289975; 20180289976; 20180295667; 20180295895; 20180296097; 20180296098; 20180296136; 20180296157; 20180296161; 20180296847; 20180300919; 20180301211; 20180303343; 20180303356; 20180303357; 20180303396; 20180304149; 20180307185; 20180310327; 20180310877; 20180310892; 20180314801; 20180315285; 20180317826; 20180325385; 20180325435; 20180325460; 20180326142; 20180329518; 20180332383; 20180333107; 20180333585; 20180333586; 20180344252; 20180344255; 20180345006; 20180348048; 20180350465; 20180352534; 20180353086; 20180353111; 20180358117; 20180368701; 20180368780; 20180369065; 20180375983; 20180376586; 20190000317; 20190000400; 20190001128; 20190007927; 20190008384; 20190008461; 20190009019; 20190015048; 20190019573; 20190028662; 20190029598; 20190029599; 20190030350; 20190036886; 20190038149; 20190038831; 20190038902; 20190046038; 20190046794; 20190053758; 20190059742; 20190059757; 20190060644; 20190061772; 20190064344; 20190068249; 20190069815; 20190073042; 20190073333; 20190073894; 20190076033; 20190076066; 20190076067; 20190076070; 20190076643; 20190077003; 20190080791; 20190082985; 20190083039; 20190083355; 20190083784; 20190090764; 20190090820; 20190091481; 20190096534; 20190099009; 20190105505; 20190108427; 20190110755; 20190116896; 20190117068; 20190117127; 20190117966; 20190122523; 20190125264; 20190126003; 20190126014; 20190130332; 20190132948; 20190133484; 20190134288; 20190137332; 20190142283; 20190146740; 20190147999; 20190150748; 20190150820; 20190150834; 20190151640; 20190154723; 20190158472; 20190159676; 20190160213; 20190167114; 20190167237; 20190168005; 20190174007; 20190175019; 20190175116; 20190175411; 20190175960; 20190175961; 20190179412; 20190180869; 20190183339; 20190183346; 20190184077; 20190190862; 20190191468; 20190192022; 20190192076; 20190192080; 20190192085; 20190192086; 20190192768; 20190197073; and 20190197861.

A new type of biometric “device” is a biometric tattoo. A team of scientists in Germany has developed permanent dermal sensors that can be applied as artistic tattoos. As detailed in the journal Angewandte Chemie, a colorimetric analytic formulation was injected into the skin instead of tattoo ink. See, Yetisen, Ali Kemal, Rosalia Moreddu, Sarah Seifi, Nan Jiang, Katia Vega, Xingchen Dong, Jie Dong et al. “Dermal Tattoo Biosensors for Colorimetric Metabolite Detection.” Angewandte Chemie (2019). See also, Graziano, Gabriella. “Functional tattoos.” Nature Reviews Chemistry (2019): 1; Russell, Ryan J., Michael V. Pishko, Christopher C. Gefrides, Michael J. McShane, and Gerard L. Cote. “A fluorescence-based glucose biosensor using concanavalin A and dextran encapsulated in a poly (ethylene glycol) hydrogel.” Analytical Chemistry 71, no. 15 (1999): 3126-3132. The pigmented skin areas varied their color when blood pH or other health indicators changed. The researchers identified and adapted three colorimetric chemical sensors that produce a color change in response to biomarkers. The first sensor was a pH indicator consisting of the dyes methyl red, bromothymol blue, and phenolphthalein. If injected into a model skin patch, the resulting tattoo turned from yellow to blue if the pH was adjusted from five to nine. The other two sensors probed the levels of glucose and albumin.

Wearable devices may be improved according to the present invention by configuring them to calculate the second derivative of the physiological parameters they are designed to measure.

### SUMMARY OF THE INVENTION

Most dynamic processes in nature may be described by the second-order differential equations or corresponding equations of other systems. These equations typically correspond to physical elements or parameters of systems that can be measured, predicted, diagnosed, and controlled. The present invention provides systems and methods for the analysis of biological systems in real-time or near real-time, for predictive analytics, diagnosis, and intervention.

Once the homeostatically-controlled systems (at least at the highest level) are considered with respect to second-order differential equations, a large body of available mathematical and engineering theory becomes immediately relevant to the analysis, predictive modelling, warning, and ultimately interventional strategies to be applied. The prior art tends to look at organisms and their systems with respect to zeroth or first-order principles, and the analysis and treatment applied based on a state, or perhaps a linear trajectory (rate). When considering second-order differential effects, each homeostatically-controlled subsystem may be considered a damped oscillator, though in most normal cases (excluding overtly cyclic processes), these are overdamped with little or no overshoot. As such, the consideration of the oscillatory nature has been ignored or considered too esoteric for practical application.

In fact, once the biological systems are properly dynamically modelled, consideration of stability criteria, secondary and tertiary effects of an intervention, or withholding intervention is facilitated. In general, for any complex biological system, only a very small number of relevant parameters may be measured, over a limited period of time, and over a limited range of conditions or contexts, and on a broader scale, on a limited population. However, in many cases, the biology is sufficiently understood such that a second-order (or higher) differential equation may be constructed, which models the system of the species (and subtype). Over the conditions or context of monitoring, the individual-specific parameters and “constants” may then be derived from a limited stream of data, thus producing a predictive model for an individual. Once a predictive model is elaborated, further data may be used to assess the quality of the model, and also to determine a change in the homeostatic state of the organism or system, i.e., a requirement to modify an otherwise accurate model. Further, an accurate model has predictive value in terms of understanding the future states of the organism or system, both in the absence and presence of intervention or change of external conditions or context.

Of particular note is that by explicitly modelling homeostatic mechanisms, the present and future stability of the state of the organism or system may be determined or predicted, and deviations from normal or predicted states for the individual attributed meaning. The prior art does not typically apply engineering-style stability analysis to biological systems, and thus fails to identify stability criteria, and how intervention(s) or proposed intervention(s) impacts these criteria. In homeostatically-controlled biological systems, a proposed goal is (in the general case) to return the system to a state where the homeostatic mechanisms maintain a stable and appropriate state of the system and organism. On the other hand, in selected cases, the issue is the opposite—the homeostatic mechanisms themselves achieve an unstable process or inappropriate state, and the goal is to defeat the improper homeostasis and/or reset its “setpoint” or guiding parameters.

For example, “chronic disease” is an inappropriate state of the organism which is relatively stable (in a colloquial sense) over time, and which appears stable from a systems analysis perspective—an “acute illness” is one where the homeostatic mechanisms have failed to sufficiently maintain the organism in a proper state. In order to monitor a chronic illness according to the present technology, one should identify the impacted systems and their interaction with other systems, and model the chronic disease as an underlying model taking into account the biological systems over the range including the “operating point” represented by the disease state and, perhaps, the operating point of the biological systems with the chronic disease defects remediated (which may be drawn from population statistics).

According to the invention, in a chronic disease state, the homeostatic controls over the biological systems may be aberrant, and therefore merely treating the symptoms to return the organism to a state wherein the measurement parameters are within the normal range may be insufficient, since the setpoint of the homeostatic mechanisms may remain displaced from the normal state. Therefore, guided by the model, and intervention may be affected that seeks to alter the setpoint, and not merely treat the symptoms of the chronic disease. By employing a higher-order dynamical model, the amount of under-compensation or overcompensation may be optimized.

On the other hand, in acute disease, remediation of symptoms may be sufficient, as the underlying homeostatic mechanisms may be fully operational. The technology may also distinguish between acute and chronic diseases, and distinguish between the underlying pathology and homeostatic compensatory mechanisms which may be intact. Because the underlying biophysics are different between an acute disease, which represents a perturbation from a normal state, and a chronic disease, which represents a set of compensatory responses that lead to a revised state different from normal but stable or semi-stable, the present technology may assist in diagnosis, distinguishing between acute and chronic phases, and assist in proposing therapy, monitoring therapy, and gauging return to health.

A particular advantage of the higher-order dynamic model is that it can predict not only a need for therapy or therapeutic intervention (or lack of need), but also the amount of therapy required with respect to an immediate response, and a delayed or rebound effect. It can determine the impact of normal activity, environment, food, or drink on the physiological state, emotional and psychological impacts, and include these factors in a time-sensitive analysis. It can further distinguish between normal responses, in contradistinction to improper responses. Typically, within physiological tolerance, simple levels (zeroth order) or rates (first-order) will not be thresholds for diagnosis or therapeutic intervention, and rather the dynamic context will also be considered. These models are typically individualized, and therefore will intrinsically account for differences between individuals.

This technology may have a particular application in new drug development, where the therapeutic index (a ratio that compares the toxic vs. effective drug's concentration) is low. In such cases, a drug may fail a clinical trial if non-dynamically optimized protocols are employed. However, by analyzing and compensating for the dynamics of the patient, a therapeutic index may be achieved, which permits safe and effective use. Thus, a style of personalized medicine is provided with an adaptive protocol dependent on patient responses and predicted patient responses, but also addressing change in patient state over the course of therapy or intervention.

By employing the tools of systems dynamics, stability criteria may be determined, and a determination made whether the patient or subject meets the stability criteria, is at risk of not meeting the stability criteria, and employing or suggesting therapy when warranted to avoid unstable states, and constraining therapy or intervention that could lead to unstable states. An unstable state is one that leads to failure of the homeostatic mechanism, an undesired homeostatic response, or significant secondary effects outside a particular biological system affected by the pathology, for example, in addition to traditional notions of system stability.

In a typical second-order dynamic system, a model is provided represented by a differential equation which, in analogy to mechanical systems, has a first-term representing a temporal scale (e.g., natural frequency), and a second term representing energy loss (damping). When such a model applies, the biological system may be assessed with respect to changes in the temporal scale (shorter or longer), and energy loss (too much, too little). Of course, cases with aberrations in both time and energy may be apparent, and cases where the model itself requires restructuring, i.e., the form of the differential equation is unsatisfactory.

The present technology is not limited to assessing poor health conditions and may be applied to the training of athletes, and other non-pathological states. For example, in sports, mechanical efficiency may be a critical determinant of performance. By measuring the dynamics of an athlete and considering the task, steps may be taken to increase performance. For example, footwear of a runner has a mass, thickness, compression, damping, lateral stability (asymmetric), etc. A gait model of the athlete may be acquired, which proposes optimal footwear, for example, in terms of damping of ground impact and timing of energy recovery. The gait model may detect changes in the athlete over time, such as fatigue, water balance, etc., and propose changes in the footwear to compensate. In some cases, the footwear itself is adaptive, and the sensors and gait model may be embedded into the footwear.

See, U.S. Pat. Nos. 4,660,242; 4,679,269; 4,829,932; 4,866,802; 5,216,594; 5,373,651; 5,615,111; 5,642,096; 5,720,200; 5,794,361; 6,230,501; 6,563,423; 6,768,246; 6,788,200; 6,833,039; 6,843,578; 6,865,825; 7,010,869; 7,059,070; 7,107,706; 7,188,439; 7,204,041; 7,219,449; 7,225,565; 7,265,666; 7,334,350; 7,395,614; 7,404,263; 7,409,256; 7,426,873; 7,493,230; 7,497,037; 7,506,460; 7,562,468; 7,579,946; 7,607,243; 7,614,166; 7,631,382; 7,671,599; 7,676,960; 7,676,961; 7,707,742; 7,735,351; 7,758,523; 7,793,430; 7,794,101; 7,816,632; 7,890,055; 7,930,131; 8,015,732; 8,028,443; 8,056,268; 8,103,802; 8,127,470; 8,141,276; 8,186,081; 8,205,356; 8,234,798; 8,250,782; 8,258,941; 8,261,468; 8,277,741; 8,291,614; 8,388,347; 8,421,822; 8,461,979; 8,499,476; 8,609,973; 8,641,220; 8,644,967; 8,656,607; 8,667,709; 8,730,034; 8,739,639; 8,740,751; 8,751,320; 8,753,275; 8,763,261; 8,857,078; 8,938,892; 8,956,228; 8,973,197; 8,982,150; 9,029,736; 9,030,335; 9,063,529; 9,089,182; 9,100,495; 9,101,260; 9,160,836; 9,192,816; 9,198,478; 9,201,413; 9,207,660; 9,215,910; 9,279,734; 9,297,709; 9,301,573; 9,326,566; 9,364,042; 9,375,047; 9,380,834; 9,388,873; 9,398,787; 9,410,691; 9,410,857; 9,414,641; 9,504,291; 9,542,706; 9,549,585; 9,572,395; 9,603,416; 9,622,537; 9,629,418; 9,635,901; 9,642,414; 9,648,926; 9,652,838; 9,655,405; 9,662,262; 9,687,577; 9,693,605; 9,709,971; 9,715,012; 9,734,527; 9,737,263; 9,743,861; 9,756,895; 9,757,302; 9,763,489; 9,763,490; 9,764,050; 9,776,041; 9,782,125; 9,810,591; 9,846,231; 9,847,006; 9,848,668; 9,854,872; 9,861,165; 9,863,823; 9,877,523; 9,879,741; 9,913,509; 9,924,760; 9,936,763; 9,961,963; 9,968,159; 9,968,238; 9,980,535; 9,984,549; 9,986,782; RE40879; RE41087; RE41102; RE41122; U.S. Pat. Nos. 10,010,129; 10,012,969; 10,013,633; 10,016,015; 10,024,660; 10,024,740; 10,034,512; 10,051,912; 10,058,146; 10,070,680; 10,070,683; 10,092,065; 10,093,112; 10,106,222; 10,111,496; 10,123,098; 10,123,679; 10,136,842; 10,151,648; 10,170,135; 10,172,396; 10,172,409; 10,172,423; 10,178,890; 10,182,608; 10,182,744; 10,188,173; 10,188,319; 10,194,714; 10,222,283; 10,226,082; 10,234,340; 10,248,985; 10,251,601; 10,277,963; 10,289,098; 10,292,453; 10,293,565; 10,299,722; 10,306,726; 10,307,081; 10,311,451; 10,314,928; 10,321,732; 10,327,672; 10,334,906; 10,339,352; 10,352,787; 10,357,078; 10,362,830; 10,370,785; 10,376,018; 20010035723; 20010045104; 20020090487; 20020121979; 20030070324; 20040177531; 20050050750; 20050053757; 20050091884; 20050183292; 20050217142; 20050261609; 20050268487; 20060103538; 20060230641; 20060230642; 20060248749; 20060248750; 20070000154; 20070000188; 20070006489; 20070011919; 20070011920; 20070021269; 20070039205; 20070039209; 20070043582; 20070142955; 20070157488; 20070180736; 20070180737; 20070247306; 20070260421; 20070271817; 20070282562; 20080000108; 20080005931; 20080097263; 20080122657; 20080167741; 20080189194; 20080197126; 20080243431; 20080254944; 20090126233; 20090135001; 20090151199; 20090178305; 20090193689; 20090199429; 20090204360; 20090241378; 20090265958; 20090284368; 20090287452; 20090313853; 20100035688; 20100037489; 20100050478; 20100090477; 20100104470; 20100122472; 20100170115; 20100187305; 20100242303; 20100289971; 20110054359; 20110056093; 20110056097; 20110094127; 20110260857; 20110285853; 20110314700; 20110314702; 20120073165; 20120086550; 20120144068; 20120167325; 20120184878; 20120234111; 20120291563; 20120291564; 20120293326; 20120311885; 20120318781; 20120324763; 20130008058; 20130019503; 20130019694; 20130064388; 20130067775; 20130114869; 20130118340; 20130185003; 20130213144; 20130213145; 20130213146; 20130213147; 20130263349; 20130265157; 20130278435; 20130278436; 20130312291; 20130326912; 20140025447; 20140033572; 20140048214; 20140059897; 20140082963; 20140123838; 20140130370; 20140144049; 20140182166; 20140201024; 20140213940; 20140214502; 20140222173; 20140244395; 20140250726; 20140259798; 20140260677; 20140260689; 20140277631; 20140277632; 20140277658; 20140285311; 20140295537; 20140305470; 20140320331; 20140327320; 20140330431; 20140331523; 20150003047; 20150011914; 20150040428; 20150048942; 20150096204; 20150096597; 20150120496; 20150137935; 20150173452; 20150177081; 20150189062; 20150189063; 20150226619; 20150254710; 20150257479; 20150260514; 20150289594; 20150289595; 20150289596; 20150296922; 20150297059; 20150313308; 20150313309; 20150335096; 20150359457; 20160000188; 20160016041; 20160025854; 20160037855; 20160044993; 20160067584; 20160074547; 20160081418; 20160081435; 20160100801; 20160106177; 20160143562; 20160180440; 20160188856; 20160195440; 20160210679; 20160219266; 20160219967; 20160220186; 20160227870; 20160227883; 20160252412; 20160260311; 20160262485; 20160262486; 20160290878; 20160299021; 20160302509; 20160302521; 20160306339; 20160309829; 20160309830; 20160309842; 20160316849; 20160334087; 20160335913; 20160341611; 20160345653; 20160345902; 20160349076; 20160366972; 20160369861; 20160374171; 20170027278; 20170038747; 20170055880; 20170058451; 20170068774; 20170068970; 20170079368; 20170085967; 20170105474; 20170105476; 20170127999; 20170135415; 20170169695; 20170172249; 20170215524; 20170215765; 20170231569; 20170238659; 20170246521; 20170255185; 20170265578; 20170265580; 20170265581; 20170265582; 20170265583; 20170265584; 20170265586; 20170265587; 20170265588; 20170265589; 20170265591; 20170265592; 20170265594; 20170270224; 20170272008; 20170273599; 20170284875; 20170290937; 20170295889; 20170303827; 20170306539; 20170308044; 20170308066; 20170308945; 20170330257; 20170332733; 20170336781; 20170340049; 20170340277; 20170351891; 20170363440; 20180008003; 20180008005; 20180020764; 20180064209; 20180070877; 20180077975; 20180094991; 20180103724; 20180110294; 20180116334; 20180122211; 20180124478; 20180125163; 20180132568; 20180132697; 20180132758; 20180146738; 20180168273; 20180169474; 20180184751; 20180199657; 20180199671; 20180199673; 20180199674; 20180206586; 20180213879; 20180228401; 20180231393; 20180242691; 20180263564; 20180271181; 20180271211; 20180271213; 20180274996; 20180279713; 20180292794; 20180295933; 20180310670; 20180325207; 20180326286; 20180338560; 20180338576; 20180339445; 20180343977; 20180343978; 20180343981; 20180361221; 20190037960; 20190037961; 20190037969; 20190039311; 20190045877; 20190052111; 20190053572; 20190082773; 20190090585; 20190090589; 20190094374; 20190116915; 20190116935; 20190116937; 20190117118; 20190133414; 20190139252; 20190150791; 20190159529; 20190159546; 20190166954; 20190174863; 20190174871; 20190175107; 20190208295; 20190208865; 20190209087; 20190213458; 20190223542; 20190231166; 20190232592; and 20190239309.

The Kalman filter is a recursive solution to the discrete-data linear filtering problem. The Kalman filter gives an estimate of the state of a dynamic system from noisy measurements. It gives a recursive minimum variance estimate of the state of the system. To be able to apply the Kalman filter to give an estimate of a state in a space, a discrete-time state-space model is needed. This consists of a state equation and a measurement equation. The state equation is given by: xₖ=Aₖ xₖ₋₁, +Bₖ uₖ+εₖ, wherein xₖ is an n-dimensional state vector, which is to be estimated; Aₖ is an n-dimensional known system matrix; Bₖ is an n by m-dimensional known input matrix; uₖ is an optional m-dimensional input vector; and εₖ represents the process noise. Because the variable uₖ is optional, it can be left out of the equation, and a new state equation that can be simplified into: xₖ=Aₖ xₖ₋₁+εₖ. The measurement equation is given by: yₖ=Cₖxₖ+δₖ, wherein yₖ is a p-dimensional measurement vector; xₖ is an n-dimensional state vector which is to be estimated; Cₖ is a p by n-dimensional known measurement matrix; and δₖrepresents the measurement error. The variables εₖ and δₖ are assumed to have zero cross-correlation and are white.

Furthermore εₖ and δₖ each has a covariance matrix (Qₖ and Rₖ respectively) and has normal probability distributions: p(εₖ)˜N(0, Qₖ), p(δₖ)˜N(0, Rₖ). The system matrix Aₖ and measurement matrix Cₖ may change with each measurement or time-step. Both of the covariance matrices Qₖ and Rₖ may also be time-variant.

With Kalman filters, the initial state is known and is represented by the state vector x₀. To be able to predict the state of the system at a time k₁, the previous estimate \hat{x}₀ is needed. Because the prediction makes use of the old estimate, before incorporating the new measurements found at the time k₁, this estimate will be called an “a priori” estimate. The a priori estimate is represented by \hat{x}₁⁻; here the hat denotes that it is an estimate, and the upper minus denotes that it is a previous estimate. So, to calculate the a priori estimate, the following equation can be formed: \hat{x}ₖ₊₁⁻=A\hat{x}ₖ. In other words, to obtain a new estimate, multiply the last collected estimate \hat{x}ₖ with A, the system matrix.

Furthermore, it is also needed to change the error covariance matrix Pₖ⁻, which is associated with the a priori estimate with every new time step. The equation showing how to calculate a new value for Pₖ⁻ is: Pₖ₊₁⁻, =APₖA^{T}+Q. Depending on the size of Q, the random process noise covariance matrix has a significant effect on the error covariance matrix. The value of Q is normally obtained before using it in the process that is to be estimated. However, this may be difficult. Q has to simulate process noise for the process to be estimated.

Given an a priori estimate \hat{x}ₖ⁻, what is then needed is an updated estimate \hat{x}ₖ; this is called the a posteriori estimate, which is obtained by combining the noisy measurement yₖ with the a priori estimate, using \hat{x}ₖ=\hat{x}+ₖ⁻ (yₖ−C\hat{x}ₖ⁻). As can be seen, the a posteriori estimate \hat{x}ₖcan be obtained by combining the a priori estimate \hat{x}ₖ⁻ with the weighted difference between the actual measurement yₖ and the measurement prediction C\hat{x}ₖ⁻. The difference (yₖ−C\hat{x}ₖ⁻) is called the residual, and reflects the discrepancy between the actual measurement yₖ and the measurement that was predicted. If the residual is zero, then it appears that the prediction of the value of the next measurement and the actual measurement are the same.

The Kalman gain Kₖ is a matrix that aims to minimize the a posteriori error covariance matrix, calculated by Kₖ=Pₖ⁻C^{T} (CPₖ⁻C^{T}+R)⁻¹. From this, it can be seen that when the measurement error covariance matrix R reaches zero, the Kalman gain will weigh the residual more heavily:

$${lim {K}_{k}}_{{R}_{k} \to 0} = {C}^{- 1} .$$

If this is the case, the actual measurement yₖ is more trustworthy. At the same time, the predicted measurement C\hat{x}ₖ⁻ is less trustworthy. If, however, the a priori estimate error covariance matrix Pₖ⁻ reaches zero, the Kalman gain will weigh the residual less heavily:

$${lim {K}_{k}}_{{P}_{k}^{-} \to 0} = 0.$$

When this occurs, when covariance matrix Pₖ⁻ approaches zero, the actual measurement is less trustworthy, and C\hat{x}ₖ⁻ is more trustworthy. The last update equation is the error covariance update equation, given by: Pₖ=(I−KₖC)Pₖ⁻. The error covariance matrix Pₖ gives a statistical measure of the uncertainty in xₖ, it is a measure of the dispersion of xₖ around \hat{x}ₖ.

The Kalman filter estimates a certain process by using a form of feedback control. First it estimates the state of the system at a given time and afterward receives feedback from incoming measurements. In other words, first, it predicts what the state of the system will be and, after receiving new measurements from sensors, these predictions will be corrected.

After obtaining new a priori estimates, the next step is to use the measurement update equations to calculate a posteriori estimates. First, the Kalman gain has to be calculated to see how much the residual will be weighed. When the Kalman gain is known, it is possible to obtain a new a posteriori state estimate. To be able to calculate the new state, the new measurement is compared to predicted measurement, multiplied by the Kalman gain, and combined with the a priori state estimate. The final step is obtaining an a posteriori error covariance estimate.

The Kalman filter has a recursive nature. In every cycle of the process, i.e., after each time and measurement update pair, the previous a posteriori estimates are used to calculate the new a priori estimates and from them the new a posteriori estimates.

The Kalman filter represents a higher-order model of the system and may be used in conjunction with the present technology. See, en.wikipedia.org/wiki/Kalman_filter. By incorporating statistical uncertainty of measurements into the model, false alarms may be suppressed, and higher quality predictions made.

Noisy sensor data, approximations in the equations that describe the system evolution, and external factors that are not accounted for all place limits on how well it is possible to determine the system's state. Kalman filter deals effectively with the uncertainty due to noisy sensor data and, to some extent, also with random external factors. Kalman filter produces an estimate of the state of the system as an average of the system's predicted state and the new measurement using a weighted average. The purpose of the weights is that values with better (i.e., smaller) estimated uncertainty are “trusted” more. The weights are calculated from the covariance, a measure of the estimated uncertainty of the prediction of the system's state. The result of the weighted average is a new state estimate that lies between the predicted and measured state and has a better-estimated uncertainty than either alone. This process is repeated at every time step, with the new estimate and its covariance informing the prediction used in the following iteration. This means that Kalman filter works recursively and requires only the last “best guess,” rather than the entire history, of a system's state to calculate a new state.

The relative certainty of the measurements and current state estimate is an important consideration, and it is common to discuss the response of the filter in terms of Kalman filter's gain. The Kalman gain is the relative weight given to the measurements and current state estimate and can be “tuned” to achieve particular performance. With a high gain, the filter places more weight on the most recent measurements, and thus follows them more responsively. With a low gain, the filter follows the model predictions more closely. At the extremes, a high gain close to one will result in a more jumpy estimated trajectory, while low gain close to zero will smooth out noise but decrease the responsiveness.

When performing the actual calculations for the filter, the state estimate and covariances are coded into matrices to handle the multiple dimensions involved in a single set of calculations. This allows for a representation of linear relationships between different state variables (such as position, velocity, and acceleration) in any of the transition models or covariances.

See U.S. Pat. Nos. 4,589,078; 4,763,282; 5,341,229; 5,626,140; 5,629,848; 5,850,352; 6,157,850; 6,236,872; 6,558,351; 6,572,545; 6,575,905; 6,949,066; 7,070,398; 7,149,320; 7,172,897; 7,215,986; 7,231,254; 7,267,665; 7,280,870; 7,312,087; 7,354,420; 7,392,079; 7,402,153; 7,414,611; 7,489,298; 7,489,299; 7,519,406; 7,519,512; 7,535,456; 7,621,876; 7,623,928; 7,716,008; 7,774,155; 7,783,442; 7,806,886; 7,853,329; 7,859,540; 7,860,676; 7,877,224; 7,927,216; 7,930,035; 7,931,535; 7,942,745; 7,974,696; 7,996,158; 8,041,536; 8,072,424; 8,089,458; 8,103,471; 8,123,696; 8,137,195; 8,157,651; 8,160,273; 8,160,669; 8,164,567; 8,169,406; 8,180,591; 8,180,592; 8,184,097; 8,216,139; 8,226,493; 8,233,958; 8,233,959; 8,237,657; 8,239,166; 8,248,367; 8,251,906; 8,257,259; 8,260,393; 8,260,558; 8,265,725; 8,267,786; 8,275,437; 8,280,475; 8,282,549; 8,290,561; 8,306,766; 8,308,563; 8,311,749; 8,311,769; 8,311,770; 8,313,379; 8,321,149; 8,348,886; 8,348,923; 8,359,545; 8,368,648; 8,373,659; 8,374,667; 8,384,668; 8,385,971; 8,386,004; 8,386,008; 8,388,530; 8,388,544; 8,396,557; 8,407,022; 8,409,003; 8,430,753; 8,433,395; 8,437,980; 8,444,560; 8,463,576; 8,463,577; 8,467,904; 8,469,886; 8,473,245; 8,475,275; 8,478,389; 8,478,557; 8,484,153; 8,484,270; 8,491,389; 8,494,829; 8,532,935; 8,543,185; 8,543,351; 8,548,770; 8,560,038; 8,565,848; 8,571,808; 8,583,402; 8,600,681; 8,608,535; 8,612,163; 8,629,836; 8,632,465; 8,657,745; 8,657,747; 8,663,109; 8,670,953; 8,672,845; 8,682,615; 8,683,850; 8,684,943; 8,686,579; 8,688,202; 8,702,515; 8,708,821; 8,708,824; 8,711,094; 8,718,753; 8,718,965; 8,727,995; 8,747,315; 8,750,955; 8,753,165; 8,755,837; 8,758,136; 8,768,313; 8,771,222; 8,777,924; 8,781,197; 8,788,007; 8,790,180; 8,795,079; 8,801,610; 8,805,110; 8,805,485; 8,814,688; 8,827,810; 8,834,271; 8,834,366; 8,842,176; 8,843,210; 8,843,321; 8,845,536; 8,849,259; 8,855,712; 8,868,377; 8,870,655; 8,886,206; 8,888,576; 8,900,142; 8,913,011; 8,915,785; 8,915,849; 8,918,178; 8,929,877; 8,937,594; 8,942,777; 8,945,017; 8,948,834; 8,961,260; 8,961,312; 8,983,628; 8,983,629; 8,986,209; 8,994,657; 8,998,815; 9,005,129; 9,008,724; 9,008,762; 9,011,248; 9,014,986; 9,039,533; 9,042,988; 9,044,671; 9,050,007; 9,055,901; 9,060,719; 9,060,722; 9,072,438; 9,082,011; 9,095,303; 9,104,965; 9,107,614; 9,107,623; 9,113,801; 9,113,823; 9,119,528; 9,119,529; 9,125,548; 9,125,577; 9,131,120; 9,131,842; 9,149,717; 9,162,148; 9,167,991; 9,173,574; 9,186,585; 9,189,739; 9,192,328; 9,204,038; 9,227,138; 9,250,716; 9,261,978; 9,271,133; 9,272,206; 9,282,925; 9,294,074; 9,298,282; 9,305,559; 9,317,743; 9,320,470; 9,320,900; 9,320,976; 9,339,202; 9,351,668; 9,358,381; 9,364,173; 9,364,609; 9,375,171; 9,393,491; 9,393,500; 9,398,872; 9,399,134; 9,400,598; 9,401,153; 9,408,549; 9,414,758; 9,415,219; 9,420,965; 9,433,376; 9,440,025; 9,446,319; 9,451,886; 9,452,258; 9,452,259; 9,456,787; 9,457,146; 9,463,277; 9,463,380; 9,468,854; 9,474,855; 9,474,962; 9,483,608; 9,486,578; 9,492,096; 9,498,155; 9,498,709; 9,498,728; 9,517,306; 9,526,430; 9,526,834; 9,565,512; 9,572,935; 9,572,936; 9,575,570; 9,577,992; 9,579,568; 9,586,004; 9,590,986; 9,597,453; 9,616,334; 9,623,179; 9,623,240; 9,629,558; 9,649,036; 9,655,565; 9,662,445; 9,675,290; 9,675,878; 9,687,194; 9,692,984; 9,700,253; 9,700,806; 9,706,956; 9,706,963; 9,706,964; 9,707,478; 9,713,766; 9,731,194; 9,737,249; 9,737,797; 9,750,441; 9,757,510; 9,770,652; 9,775,543; 9,779,502; 9,795,737; 9,797,880; 9,801,571; 9,801,577; 9,804,150; 9,811,730; 9,814,973; 9,826,922; 9,827,372; 9,836,896; 9,849,239; 9,852,736; 9,853,976; 9,861,887; 9,868,332; 9,872,652; 9,878,096; 9,886,845; 9,897,985; 9,900,669; 9,907,909; 9,913,599; 9,918,183; 9,918,646; 9,931,578; 9,936,910; 9,943,247; 9,943,267; 9,946,356; 9,975,196; 9,993,724; 9,999,728; RE45905; U.S. Pat. Nos. 10,002,233; 10,010,790; 10,022,624; 10,025,973; 10,026,410; 10,028,706; 10,031,002; 10,045,713; 10,045,720; 10,046,113; 10,049,187; 10,061,911; 10,089,446; 10,098,549; 10,119,956; 10,130,767; 10,137,365; 10,143,409; 10,143,426; 10,154,789; 10,155,170; 10,159,897; 10,178,973; 10,179,283; 10,188,334; 10,188,953; 10,194,850; 10,234,942; 10,236,006; 10,238,978; 10,261,069; 10,275,640; 10,293,184; 10,300,303; 10,300,374; 10,307,671; 10,307,683; 10,327,708; 10,332,315; 10,333,932; 10,339,654; 10,349,872; 10,357,210; 10,363,420; 10,369,463; 20020099282; 20020111547; 20020128544; 20020177135; 20030130616; 20030195404; 20040034295; 20040039243; 20040053290; 20040193025; 20050069162; 20050135948; 20050240242; 20050256385; 20050267342; 20060160205; 20060211942; 20060224109; 20070088226; 20070098600; 20070123779; 20070146371; 20070161919; 20070162086; 20070162164; 20070167991; 20070173761; 20070189921; 20070208212; 20070270668; 20080119900; 20080188796; 20080202927; 20080208538; 20080242553; 20080260212; 20080314395; 20090012766; 20090022336; 20090057147; 20090112478; 20090149148; 20100023089; 20100028984; 20100048242; 20100114015; 20100138379; 20100160807; 20100168538; 20100268477; 20100274102; 20100285981; 20100305545; 20100324382; 20110004110; 20110029269; 20110034176; 20110044524; 20110106453; 20110143811; 20110180425; 20110184267; 20110212717; 20110301436; 20120022336; 20120022350; 20120022384; 20120022805; 20120022844; 20120065507; 20120083705; 20120083714; 20120083715; 20120083716; 20120084053; 20120084054; 20120203131; 20120226471; 20120226472; 20120258776; 20120277545; 20130073254; 20130073255; 20130080113; 20130096843; 20130109946; 20130151196; 20130158369; 20130158473; 20130158503; 20130165901; 20130179382; 20130190638; 20130217440; 20130238049; 20130238050; 20130245401; 20130245462; 20130245711; 20130245712; 20130245981; 20130268236; 20130273968; 20130282322; 20130282646; 20130289424; 20130295894; 20130297220; 20130317580; 20140005633; 20140039383; 20140051949; 20140052091; 20140052092; 20140052093; 20140052094; 20140052095; 20140058221; 20140066884; 20140066885; 20140066886; 20140066887; 20140066888; 20140066889; 20140066892; 20140067278; 20140080428; 20140088393; 20140108020; 20140114278; 20140118138; 20140118166; 20140128803; 20140142958; 20140163517; 20140180240; 20140236536; 20140257437; 20140275854; 20140275886; 20140276119; 20140276554; 20140276555; 20140288436; 20140288438; 20140288620; 20140303552; 20140323897; 20140343867; 20140371913; 20150011194; 20150022675; 20150032178; 20150080756; 20150088024; 20150100038; 20150134268; 20150146939; 20150148700; 20150164377; 20150164432; 20150190636; 20150190637; 20150196229; 20150196256; 20150220700; 20150250429; 20150304797; 20150358525; 20160007890; 20160029931; 20160029966; 20160038673; 20160058302; 20160058329; 20160058332; 20160058333; 20160058356; 20160058370; 20160058371; 20160058372; 20160113838; 20160135706; 20160162662; 20160166156; 20160192865; 20160193679; 20160220151; 20160232726; 20160234174; 20160235352; 20160239084; 20160241554; 20160242700; 20160256112; 20160256629; 20160279410; 20160287184; 20160293172; 20160324450; 20170027523; 20170065230; 20170076068; 20170079596; 20170111359; 20170113046; 20170119261; 20170119968; 20170133022; 20170135633; 20170136842; 20170147722; 20170147803; 20170156593; 20170156606; 20170165425; 20170181671; 20170181677; 20170209055; 20170215028; 20170216625; 20170216627; 20170224291; 20170236407; 20170245767; 20170252513; 20170272842; 20170273606; 20170281867; 20170300741; 20170311897; 20170311902; 20170325736; 20170340260; 20170365101; 20180035951; 20180036147; 20180043095; 20180043096; 20180055376; 20180085011; 20180085040; 20180092573; 20180136486; 20180139518; 20180147349; 20180158266; 20180160985; 20180162186; 20180177963; 20180192900; 20180198785; 20180211673; 20180233028; 20180242891; 20180268237; 20180293430; 20180296142; 20180317808; 20180333051; 20180333535; 20180344220; 20180350468; 20180353139; 20190012608; 20190013090; 20190059826; 20190072917; 20190076600; 20190104989; 20190117115; 20190118283; 20190159737; 20190172197; 20190175079; 20190175080; 20190175082; 20190180153; 20190180438; 20190188895; 20190192009; 20190201037; 20190201038; 20190201039; 20190201040; 20190201042; 20190201043; 20190201046; 20190201047; 20190201075; 20190212323; 20190216350; 20190244347; and 20190244348.

Four use cases are considered, though the technology is not so limited. In the first use case, a system within a normal organism is modelled by its dynamic response parameters, e.g., the equation form and parameters of a higher-order dynamic response equation. This model is derived from organismal biology and sensor measurements of the individual. The biological system in the individual is monitored for deviation of the organism from the model, with analysis to determine whether the perturbation is due to a change in the organism from the model, which may be indicative of pathology, or a stimulus that excites the normal physical mechanisms.

The second use case is where the individual has, or is suspected of having, pathology, and the system and method are applied to assist in the diagnosis and proactive monitoring. This use case differs from the normal individual, in that the system model cannot a priori be predicted to correspond to the general population. Given the homeostatic nature of the system, deviations in sensed physiological parameters may represent aberrant physical systems, aberrant stimuli, or both. In this case, where pathology is diagnosed or suspected, a particular motivation for the technology is to determine a change in the state of the individual, or responses that, though within the normal homeostatic process, are nevertheless actionable.

The third use case involves athletes and performance-oriented individuals, who are intentionally exposing themselves or enduring extreme stimuli. In this case, the underlying system is presumed “normal” or in good or high fitness, but the biological system is being externally stressed, such as by exercise, altitude, dehydration, hypoxia, lactic acidosis, etc. In these cases, often the motivation for the application of the technology is to improve performance given the nature of the task. For example, a marathon runner or bike in a race wishes to finish the race quickest, while adopting acceptable risk. How much liquid should be consumed, when, what type, what temperature?What is the target heart rate?What is the optimum gait pattern, pedaling rate, gear, etc.?

The fourth use case involves healthy adults in their senior years. In this case, while the aging is not in and of itself is considered a pathology, nevertheless, the “elasticity” of the systems is diminished, self-regulatory mechanisms in charge of maintaining homeostasis of different systems become progressively sluggish (or aged) generally leading to an extension of the recovery time needed to bring the system under stress back to normal state. In this case, where pathology is not diagnosed but is expected as a result of unexpected stress, such as a fall or a viral infection, a particular motivation for the technology is to determine the degree of loss of “elasticity,” i.e., loss of efficiency of self-regulatory mechanisms in their ability to restore homeostasis.

In each of these systems, the homeostatic system within the individual is modeled with a dynamic equation and/or statistical model, based on actual sensor data from the individual, and the parameters of the dynamic equation serve as inputs or outputs. A consistency analysis is performed where feasible, based, for example, on partially redundant sensor data and/or population statistics. The sensor data from the individual are principally analyzed as state variables within the model, to predict errors in predictiveness of the model, as well as predicted state of the individual. For example, while the present technology proposes to supplement level and rate parameters with acceleration/higher-order dynamic parameters for assessment of health or physiological status, the level and rate may remain as important data; for example, levels of glucose, sodium, calcium, temperature, osmolarity, heart rate, etc. remain important limiting criteria, with useful thresholds. Including dynamic characteristics in the analysis also permits earlier prediction of likely excursions beyond tolerable limits, and also the prediction of acceptable deviations even if extreme, for example. Analyzing the model itself (distinct from the full state of the individual) also permits assessment and, for example, would assist in chronic therapies and health programs.

The use of mathematical derivatives also separates the dynamic pattern from initial conditions noise. Whatever physiological parameter is being considered, discovering its acceleration, i.e., the second derivative over time, reveals the dynamic of the system—the “force” that is acting on the system, causing the system to “accelerate” the evolution of the given parameter over time. In this context, the derivatization tends to normalize members of the population, since, while individual operating points may vary, in large part the underlying mechanisms are the same for different individuals of the same species, and eliminating the initial conditions from consideration facilitates comparison of the dynamics of the underlying system.

If we consider a homeostatically-regulated physiological system, then the value of that system may be normal, while the underlying physiological system is stressed. Thus, measurement of the level alone will only reveal perturbation once the homeostatic ability is overcome or defective, while underlying influences may be obscured. Further, measurement of feedback variables may reveal perturbation even where the controlled variable is in a normal range. For example, the accelerating value of Prostate-Specific Antigen (PSA) may indicate an early stage of malignant disease of the prostate, while the value may still be within the normal range. If one considers the slope or trend of the velocity of change, one must also consider within the homeostatically controlled system what the limit of the trend is, which then requires defining an interval of interest, since the trend may not be enduring.

However, when one looks at the second derivative of any variable within the system, the control dynamics of the system emerge. As one looks to even higher derivatives, these tend to be dominated by noise, and even sharp changes in a third derivative are not necessarily correlated with physiologically important events related to health.

Thus, the present invention relates to the extraction and analysis of dynamic variations in physiological parameters reflecting the effects of self-regulatory feedback loops within the system. In a continuous system, the dynamic variation corresponds to the second derivative, while in other systems, the corresponding concept may be adopted as the dynamic process which restores the system to a predetermined state. Note that simple first-order feedback will reduce error, but not restore the system, while homeostatic systems may fully or even overcompensate for perturbations, requiring a higher-order control. These systems have a time characteristic within their dynamics, with a response delay, and in some cases, a predictive response.

When analyzing such systems, the level and change rate of the parameter are not irrelevant, but can often be considered using traditional analysis. However, with higher-order analysis, the level and rate may be better interpreted in a broader, dynamic context. For example, hyperthermia may be quite abnormal, but if the dynamic physiological response to the hyperthermia is sufficient, the temperature alone may not represent the best basis for determining the action. On the other hand, subacute hyperthermia with a poor dynamic homeostatic response may be cause for alarm. Similarly, the issues are the same for most, if not all, homeostatically controlled variables and also the effector variables, especially if correlations with the controlled variables are considered, and alternate hypotheses analyzed and excluded. Thus, the analysis of the second-order dynamic system has fundamental distinctions from level and rate analyses, since it is better mapped to the underlying physiology, and the physiological defects that correspond to disease as differentiated from natural (and not pathological) responses.

Modern medicine often treats symptoms that represent natural responses, and thus can actually defeat the body's ability to restore itself, and therefore weaken the systems or the organism. Likewise, modem medicine tends to withhold treatment of subclinical symptoms, considering early intervention to correspond to overtreatment. Such medical decisions are often made based on the static snapshot of a particular system, which may be a poor representation of the underlying dynamics of a live organism. However, by extracting the underlying dynamics of the physiological systems, pathology and homeostatic responses may be better distinguished, with therapy targeted and titrated based on the pathology to avoid interference with normal and helpful natural responses.

On the other hand, in some cases, natural responses may be harmful; for example, autoimmune and inflammation responses, and, therefore, early intervention once these are detected may preempt symptoms.

One goal of the present invention is to detect and employ a second derivative (or discrete and/or statistical analogs thereof) or (at least) second-order differential equation of one or more physiological parameters, to better forecast the evolution of physiological states or symptoms and underlying disease, distinguish actionable patient states, and guide therapeutic intervention or withholding thereof.

According to the aspects illustrated herein, the invention includes a method of forecasting change in the physiological parameters of the human (or animal) body, based on an analysis of the second derivative and, more generally, higher-order (order >1) dynamic responses. In cases where multiple sensed parameters are available, statistical techniques may be employed, at various orders of dynamic evaluation, to produce more sensitive or earlier, accurate or precise, more reliable, or otherwise improved information upon which to alert a patient or health care provider of a need to change or consider changing therapy.

As noted above, the analysis need not be in a continuous-time, analog domain, or a discrete-time digital data domain (time series), and may be in a hybrid domain or other domain. Various parameters may be considered, using statistical techniques, or other combination techniques to increase the quality of diagnosis or treatment.

This technology may be used, for example, to detecting a change in patient status that may require medical attention, or on the other hand, a relaxation of strict medical constraints or treatment regime.

This technology may also be used to estimate the biological age of a particular organ, system, or subject as a measure of biological systems to return to homeostasis after stress, which depends on the effectiveness of self-regulatory feedback mechanisms. This biological age may have predictive value to assess future health and progression of chronic disease. The biological age, in turn, may be used to titrate therapy or assess test results.

The method, for example, may employ a traditional second derivative of a continuous function reconstructed through curve-fitting, or periodically oversampled smooth time function representing a physiological parameter. The method may also employ finite differences and other similar techniques of discrete analysis. However, the method may also encompass signals which are aperiodically sampled at discrete time intervals, or otherwise fail to meet definitions for a smooth, continuously differentiable function.

For example, a glucose measurement of a patient may be taken every 5 minutes, with a sampling duration of 10 seconds, and a time imprecision of measurement of 10 seconds (+/−5 seconds). In fact, the underlying blood glucose levels may vary with a half-life of 15 minutes for IV glucose. (Hahn, Robert G., and Thomas Nyström. “Plasma volume expansion resulting from intravenous glucose tolerance test.” Computational and mathematical methods in medicine 2011 (2011), www.hindawi.com/journals/cmmm/2011/965075/.) As a result, the blood glucose level is oversampled, and, therefore, a curve may be optimally fitted to, e.g., 5 data points (-20 minutes, −15 minutes, −10 minutes, −5 minutes, 0 minutes), with a weighting of more recent times over prior times. The second derivative (or second-order finite difference) is calculated for the terminal value as a basis for determining a prospective need for insulin, glucose, or fructose.

The present technology also preferably normalizes the signal, applying statistical analysis to the higher-order difference resulting from the signal, predicting timing offset and the effect on the higher-order difference, and employing an absolute level of the physiological parameter sensitivity in the analysis. For example, when considering a parameter in which elevation over baseline is problematic, an alert threshold for the higher-order difference is suppressed near the baseline and becomes more sensitive as the normalized value of the signal increases. If the parameter has known dynamics, a physical model reflecting those dynamics may be employed to create an expectation according to the model or, in the absence of the established model, to a hypothesis. So long as the expectation agrees with the hypothesis, a predicate for the hypothesis is validated. When a deviation of the expectation of the higher-order difference of the signal occurs, the underlying hypothesis should be investigated and the cause of deviation considered. Depending on the urgency of the detection of a change in status, a statistical temporal analysis of the signal, signal difference, and higher-order difference over-time, may be analyzed to filter unattributed variance, e.g., noise.

In cases where an immediate response to a change in the patient's state is required, a statistical temporal analysis may only be backward-looking, and the system must remain sensitive to immediate changes.

The methods of calculating the diagnostic values are provided, and hence, medical devices and implants and other wearable biometric devices can be made using this technology. The devices need not be wearable, and in some cases, remote sensing such as imaging/video, radar, lidar, sonar, or another remote or non-contact physiological sensing may be employed.

The present technology considers homeostatically controlled biological systems through the tools of dynamic system analysis and therefore addresses their implied stability and instability criteria, fragility, and interconnection with other dynamic systems. Therefore, a measurement of a biological parameter over time is considered with respect to its dynamic impact on and reflection of the health of the organism, with application toward diagnosis and prognosis, on the one hand, and predicting the need for and optimization of treatment, on the other hand. By considering the state of the biological system and various stability criteria, in view of the natural homeostatic mechanisms available (and consideration of any defects in the natural mechanisms), treatment may be applied to supplement the natural mechanism, in the general case, such as strengthening and/or augmenting self-regulatory mechanisms and feedback loops or overriding that mechanism where an aberrant response occurs (such as in autoimmune response) or where therapy requires overcoming that mechanism. By considering the dynamic response of the system and the organism, the correct treatment may be applied over time, in the case of therapeutic use, or prediction of system and health state accurately made in the case of monitoring.

It is posited that the second derivative of a physiological parameter in a biological system presents an important diagnostic and predictive value. The present invention provides for a method and a device for periodic sampling of physiological parameters and approximating the second derivative over time of the collected data to use for therapeutic, diagnostic, and predictive purposes. The physiological parameter of interest may be measured under a presumption that it is a smooth function over time, which has at least a first derivative and a second derivative over time.

An obvious and intuitive way of dealing with discrete data resulting from sampling a smooth process is to interpolate the data on a simple (e.g., polynomial) function, which allows the application of regular differentiation by taking the first and second derivative of the interpolated function. However, when automated processors are employed, this intuitive approach is not necessary, and more direct calculations may be applied.

In one preferred embodiment, the present invention discloses a method of diagnosis using biometric devices periodically taking a sample of a biological specimen, measuring a level of at least one physiological parameter of the sample, and then storing a value representing the level of said at least one physiological parameter in a non-transient long-term memory. The method further comprises ascertaining a change over time (first derivative) of said at least one physiological parameter and then ascertaining a rate of change (second derivative) of said at least one physiological parameter.

In another preferred embodiment, the present invention discloses a method of retrieving data representing the level of at least one physiological parameter over a period of time, ascertaining a change over time of said at least one physiological parameter by approximating a first derivative of the level over time by calculating finite differences and ascertaining the rate of change of said at least one physiological parameter by approximating a second derivative of the level over time by calculating the second finite difference, wherein the finite difference can be, for example, a forward finite difference, a backward finite difference, or, a center finite difference.

In a preferred embodiment, the present invention discloses a method of diagnostics comprising steps of measuring a level of at least one physiological parameter in a subject (human or animal), approximating the first derivative and the second derivative of said at least one physiological parameter over time, to determine a health hazard or risk and sending an alert to the subject or a healthcare provider quantitatively dependent on the health hazard or risk.

In a preferred embodiment, the present invention discloses a biometric device for measuring at least one physiological parameter in a subject (human or animal) comprising a housing at least partially containing the device, at least one sensor for measuring said at least one physiological parameter, a non-volatile memory operatively connected to said at least one sensor and an automatic processor operatively connected to said at least one sensor, where the processor is configured to calculate at least one second-order temporal parameter of said at least one physiological parameter, the second-order parameter being, for example, a second derivative, a second-order equation, or the like. The biometric device may be an implantable or a wearable device. Alternatively, the biometric device may be external to the body of the subject and obtain a measurement of said at least one physiological parameter remotely, such as, for example, using a laser beam for optical tomography and/or spectroscopy, a camera or video camera, a radar, lidar, or sonar sensor, electrophysiological sensors, microphone, inertial or absolute position sensors, or other types of sensors.

In a preferred embodiment, a biometric device is provided for measuring at least one physiological parameter, comprising a communication module configured to send an alert to a person and/or a healthcare provider, dependent on a deviation of the measured at least one physiological parameter from that predicted by an at least second-order model of the person. For example, the alert may be sent when the future value of said at least one physiological parameter, computed using a value of the physiological parameter, its first derivative, and the second derivative, is predicted to will exceed a predetermined or adaptively determined threshold value indicating an abnormal condition or a medical condition, for example at any future time, within a predetermined time interval, or an adaptively determined interval. The device may calculate reliability of a prediction, and weight the expected excursion of the value of the physiological parameter from its nominal value (i.e., health risk if the prediction is accurate), and the statistical likelihood that predicted the excursion would occur. For example, a low risk of a large excursion may justify an alert, while a modest risk of a small excursion may not warrant an alert. Clearly, a high risk of a large excursion justifies an alert, and a predicted lack of excursion does not justify an alert.

A biometric device may be provided for measuring at least one physiological parameter in a person, further comprising at least one actuator configured to perform an action when an at least second-order model of the biological system of the person associated with said at least one physiological parameter predicts an aberration corresponding to a health risk, which may be, for example, that the second derivative exceeds a predetermined threshold value or if the future value of said at least one physiological parameter computed using the absolute value, the first derivative and the second derivative will exceed a predetermined threshold value.

A biometric device may be further provided for measuring at least one physiological parameter in a person, further comprising a lab-on-a-chip configured to test a plurality of physiological parameters, and a controller configured to control the lab-on-a-chip (or lab on paper) and read outputs therefrom. The controller may be an FPGA, or traditional microprocessor with peripheral input-output interfaces.

It is, therefore, an object to provide an apparatus for analyzing a biological system, comprising: an input port configured to receive quantitative information reflecting at least one physiological parameter of the biological system over time; a processor configured to: perform a dynamic analysis on said at least one physiological parameter dependent on at least one derivative of the second-order of said at least one physiological parameter of the biological system; predict a future state of said at least one physiological parameter or of the biological system based on the dynamic analysis; analyze the predicted future state according to at least one decision criterion; and an output port configured to generate at least one alert selectively dependent on the analysis.

The physiological parameter may be selected from at least one of a heart rate, a cardiac stroke volume, a blood pressure, a respiratory rate, respiratory volume, and a body temperature.

The apparatus may be an implantable device or a wearable device,

The quantitative information may be derived from at least one of a motion sensor, a proximity sensor, a barometric sensor, a pressure sensor, a thermometer, a galvanic skin sensor, a photoplethysmography sensor, an electroencephalogram sensor, an electromyogram sensor, a blood flow sensor, a bio-impedance sensor, an altimeter, optical sensor, and a ultraviolet light sensor.

The processor may be further configured to communicate with an online server.

The apparatus may further comprise at least one actuator responsive to the analysis of the predicted future state.

The processor may comprise an FPGA.

The apparatus may further comprise a lab-on-a-chip configured to test a plurality of said at least one physiological parameter. The apparatus may further comprise a lab-on-a-paper.

It is also an object to provide a method for measuring at least one physiological parameter of a user and sending an alert to the user and a healthcare provider indicating abnormal health condition of the user, the method comprising: sampling a biological process; measuring a value of the sampled biological process; storing the measured value in a nonvolatile memory; determining parameters of a dynamic model equation of at least second-order predictive of the biological process; determining a deviation of the biological process from a prediction based on the dynamic model; and applying a decision criterion to the determined deviation; producing an output selectively dependent on the applied decision criterion.

The biological process may be a process selective from the group consisting of a neurological process, a muscular process, a skeletal process, a digestive process, a cardiac process, a respiratory process, a renal process, a dermal process, a reproductive organ process, a hemodynamic process, an electrochemical process, a blood chemistry process, and a vascular process.

Said at least one physiological parameter may be selected from the group consisting of at least one of a heart rate, a cardiac stroke volume, a blood pressure, a respiratory rate, respiratory volume, and a body temperature

The decision criterion may be a threshold value determined according to the normal range of the physiological parameter for a healthy body condition.

The output may comprise an alert sent wirelessly in the form of a message or text, an alert that may be displayed on display proximate to the user, an alert sent to a remote the health care provider for the user, or an alert communicated through an online server.

It is a further object to provide a method for measuring at least one physiological parameter of a user and sending an alert to the user and a healthcare provider indicating abnormal health condition of the user, the method comprising: determining at least one physiological parameter by a sensor; storing a value representing the determined at least one physiological parameter in a non-volatile memory; ascertaining an at least second-order predictive model for said at least one physiological parameter and storing the at least second-order predictive model in a non-transitory storage database; determining a consistency of the at least second-order predictive model with the user; determining a predicted deviation of the user from a reliable at least second-order predictive model; and issuing an alert based on a predicted deviation and reliability of occurrence of the predicted deviation.

It is a still further object to provide a biometric device for forecasting at least one physiological parameter in a subject, the device comprising: at least one probe configured to measure said at least one physiological parameter; a non-volatile memory configured to store values of said at least one physiological parameter; a processor operatively coupled with the non-volatile memory and configured to: determine an initial value of said at least one physiological parameter at a given time t₀; determine an initial value of a first derivative of said at least one physiological parameter at the time t₀; approximate a first derivative of said at least one physiological parameter over time; approximate a second derivative of said at least one physiological parameter over time; compute the future value of said at least one physiological parameter at a future time t₁; compare the computed future value of said at least one physiological parameter at the future time t₁ with a predetermined threshold value; and an output operatively coupled to the processor configured to generate at least one signal if the computed future value of said at least one physiological parameter at the future time t₁ is equal or greater than the predetermined threshold value.

The biometric device may be one of a wearable device, an implanted device, and a remote device.

The subject may be one of a human subject and an animal subject.

Said at least one physiological parameter may be regulated by a homeostatic system. Said at least one physiological parameter may be an effector of a homeostatic system.

The first derivative and the second derivative are approximated by fitting a smooth curve on a dataset of values of said at least one physiological parameter; and taking respectively first and second derivatives over time of the smooth curve. The first derivative and the second derivative are approximated by computing a first finite difference and the second-order finite difference, wherein the finite difference may be one of a backward derivative, a forward derivative, and a centered derivative.

The future time t₁ may be the time for which a future value of said at least one physiological parameter needs to be predicted.

The threshold value may be a critical value indicating the mortal danger of the subject, a critical value indicating an irreversible change in a biological system upon which the homeostasis cannot be naturally reversed, a critical value exceeding a homeostatic range, indicating a need for medical intervention, or a value of concern indicating likely disease.

The biometric device may further comprise the actuator operatively coupled to the output; the actuator configured to be triggered by the signal. The actuator may be configured to deliver one of a pharmacological agent and an electric stimulus.

It is another object to provide a system for analyzing at least one physiological parameter of a user and sending an alert to the user and a healthcare provider indicating the medical condition of the user, the biometric device comprising: a housing containing the biometric device; a non-volatile memory operatively connected to said at least one sensor; said at least one sensor configured to measure at least one physiological parameter; a processing module operatively connected to the non-volatile memory, wherein the processing circuitry comprises: a measuring module configured to receive and store the measured physiological parameter in the non-volatile memory; a calculation module configured to determine a first derivative and a second derivative of said at least one physiological parameter; a determination module configured to determine a threshold level of said at least one physiological parameter; a configurable analysis module configured to compare a value dependent on at least the second derivative with the determined threshold level; and a communication module, configured to generate the alert to the user and to the health care provider if the value exceeds the threshold level.

It is also an object to provide a method of forecasting an evolution in time of at least one physiological parameter in a subject, the method comprising: sampling values of said at least one physiological parameter over a period of time; creating a dataset of values of the sampled values; approximating a first derivative of said at least one physiological parameter from the dataset of values; approximating a second derivative of said at least one physiological parameter from the dataset of values; and using at least the second derivative of said at least one physiological parameter to forecast the value of said at least one physiological parameter beyond the time period.

The subject may be one of a human subject and an animal subject.

The sampling values of said at least one physiological parameter over the period of time may be a periodic sampling or an aperiodic sampling.

The period of time may be longer than, shorter than, or approximate a natural period of fluctuation of said at least one physiological parameter.

The dataset of values may be organized in a relational database, wherein the table of the values of said at least one physiological parameter may be linked with a table of time points at which each of the values was sampled, a table of objective physiological data for the subject representing time points within a time range at which the values of said at least one physiological parameter were sampled, a table of subjective health data for the subject representing time points within a time range at which the values of said at least one physiological parameter were sampled, a table of physiological data other subjects that that from which the values of said at least one physiological parameter were sampled, or a table of physiological data the subject acquired over a time range different from that from which the values of said at least one physiological parameter were sampled.

The method may further comprise fitting a curve on values of the dataset, wherein approximating the first derivative of said at least one physiological parameter may be done by taking the first derivative of the curve, and approximating the second derivative of said at least one physiological parameter may be done by taking the second derivative of the curve. The curve may be fitted to include each value of the dataset or may be fitted to exclude at least one value of the dataset. The curve may be fit by a statistical process responsive to a curve-fitting error. The curve may be fit by an artificial neural network. The curve may be a Bezier curve.

Approximating the first derivative of said at least one physiological parameter may be done by calculating a first-order finite difference on the dataset. The first-order finite difference may be one of a forward finite difference, a backward finite difference, and a centered finite difference. Approximating the first derivative of said at least one physiological parameter may be done by calculating a second-order finite difference on the dataset. The second-order finite difference may be a five-point stencil.

The method may further comprise setting at least one threshold level of the forecasted value, and producing an output dependent on a relationship of the forecasted value and said at least one threshold level of the forecasted value.

The method may further comprise evaluating a function dependent on at least the second derivative with respect to at least one threshold level.

The method may further comprise issuing an alert dependent on the evaluation.

Said at least one physiological parameter may be measured in the subject by a biometric device having a sensor, the method further comprising: measuring the values of said at least one physiological parameter with the sensor; storing the measured values in a non-transient memory; maintaining the dataset of the values in the non-transient memory of the device; approximating the first derivative and the second derivative with an automated processor, and storing the first derivative and the second derivative in the non-transient memory of the device; and outputting at least the value of the second derivative from the biometric device.

A further object provides a method of diagnosing a physiological status of a biological system in a subject, the method comprising: sampling a plurality of values of at least one physiological parameter over a period of time representing the physiological status; storing the plurality of values of said at least one physiological parameter in a database; approximating an at least second-order equation modelling the physiological status of the biological system in the subject over time; and using at least the second-order equation and successive values of said at least one physiological parameter to diagnose the physiological status of the biological system in the subject.

The diagnosis of the physiological status may be selected from the group consisting of a healthy steady-state, an unhealthy steady state, an unstable state. The healthy steady-state may indicate the diagnosis of the healthy biological system, the unhealthy steady state indicates a chronic disease of the biological system, and the unstable state indicates an acute condition. The diagnosis may be one of an acute condition and a chronic disease, and the method further comprises the step of sending an alert with the diagnosis to the subject and/or a healthcare provider for the subject.

A still further object provides a method of monitoring health of a subject, comprising: acquiring data representing a health-responsive parameter; filtering the data with a statistical filter to produce a differentiable output; providing an at least second-order predictive model, the model comprising a statistical decision boundary with respect to both probability of the existence of a future state and a predicted severity of the predicted future state; determining an output, based on the statistical decision boundary of the at least second-order predictive model, and a time-series of the data.

The statistical filter may comprise a Kalman filter.

The at least second-order predictive model may comprise at least one second-order differential equation.

The statistical decision boundary may comprise an empirically-determined prognosis algorithm, dependent on normalized population data for the subject, an artificial neural network trained based on clinical data, an expert-defined set of rules, or an adaptively-determined algorithm, dependent on at least prior data from the subject.

The data representing a health-responsive parameter may be acquired through a non-contact sensor, a contact sensor, an electrophysiological sensor, a chemical sensor, a cardiac sensor, a sensor through a wireless communication link, or an implantable sensor.

The at least second-order predictive model may be adaptively updated.

The data representing a health-responsive parameter may comprise a plurality of different health-responsive parameters.

The data representing a health-responsive parameter may be acquired periodically or aperiodically.

The data representing the health-responsive parameter may comprise a plurality of different health-responsive parameters. The subject may have a chronic illness for which the at least second-order predictive model differs in form from normal members of the subject's species. The subject may have an acute illness for which the at least second-order predictive model may be in common form with normal members of the subject's species.

The method may further comprise acquiring data from the subject to establish initial conditions for the at least second-order predictive model.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed description with reference to the drawings, in which:

FIG. 1 illustrates a biometric device that is in accordance with at least one of the various embodiments;

FIG. 2 is a flowchart showing a process for a method of diagnostic and generating alert in case of medical condition in accordance with one of the embodiments of the invention; and

FIG. 3 illustrates a flowchart showing a process for a method of diagnostic and generating alert in case of medical condition in accordance with another embodiment of the invention.

FIGS. 4-9 show flowcharts of processes according to various embodiments of the invention.

### DETAILED DESCRIPTION OF PREFERRED EMBODIMENTS

Referring now more particularly to the drawings, advantageous features of a biometric device like the device has been described that works on an efficient procedure to measure different physiological parameters and generate an alert indicating abnormal body conditions with embodying the principles of the present invention.

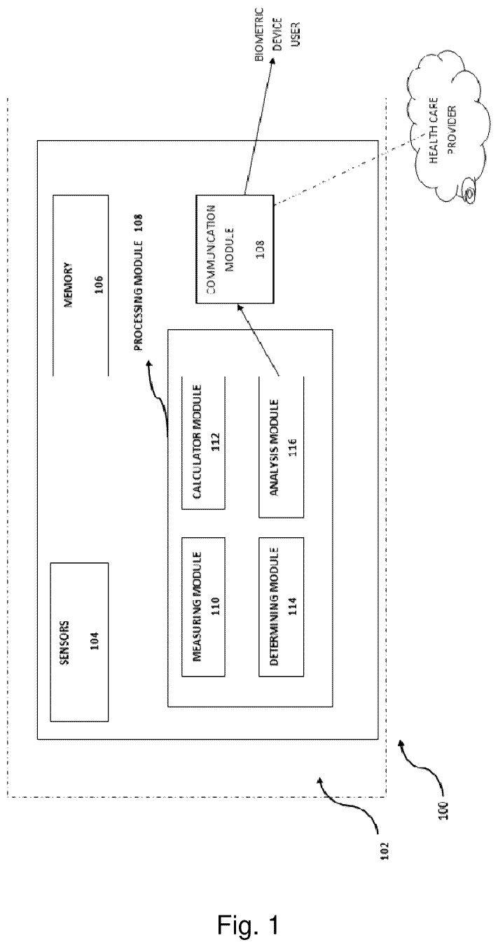

FIG. 1 presents a schematic illustration of a biometric device (100) in accordance with one of the embodiments of the present invention. The biometric device (100) may be wearable or implantable. This device provides data or analysis for a health care provider or a simple user of the device. The biometric device (100) further comprises a housing (102) for at least partially containing the biometric device. A non-volatile memory (106) or non-transitory storage media may be present for storing data related to the measurement of at least one physiological parameter. A sensor (104) for measuring at least one physiological parameter is provided, either internal or external to the device. The sensor (104) may be any suitable known physiological parameters sensor or a sensor for sensing a derivative or indirect value associated with a physiological parameter.

For example, and without limitation, the sensor may be selected from a motion sensor, a proximity sensor, a barometric sensor, a pressure sensor, a thermometer, a galvanic skin sensor, a pulse oximeter, an ion or solute sensor, an electrochemical and/or enzyme-linked electrochemical sensor, a spectrophotometric or spectrofluorometric sensor, an NMR or ESR sensor, a photoplethysmography sensor, an electrocardiogram sensor, an electroencephalogram sensor, an electromyogram sensor, a blood flow sensor, a bio-impedance sensor, an altimeter sensor, optical sensor, an infrared sensor, or an ultraviolet light sensor.

The biometric device (100) may further comprise or communicate with an external processing module (108) operatively connected to the non-volatile memory (106) for executing a series of steps to execute an algorithm for analyzing or preliminarily analyzing data from the sensor.

The processing module comprises a measuring module (110) configured for storing and receiving the measured physiological parameter in the non-volatile memory, an analysis module (112) designed to determine higher-order dynamic characteristics (e.g., a first derivative and a second derivative) of said at least one physiological parameter, a configurable analysis module (116) to determine whether the higher-order dynamic characteristic (e.g., second-order formulation) conforms with the predetermined or adaptively determined criteria, a communication module (118), to selectively communicate a message dependent on the higher-order dynamic characteristic, e.g., to generate an alert signal to the user and/or to the health care provider. The health care provider may be a doctor, surgeon, or any medical specialist who is located at a remote location and is connected to the device (100) directly or by means of an online server.

The analysis may provide a weighted analysis with discounting of values of past physiological sensor readings, and/or provide a suite of calculations taking into consideration all or some of the past data. For example, an analysis emphasizing the last parameter received will be single-ended, while a retrospective analysis of the prior 10 or 100 data points may be effectively double-ended. While this double-ended analysis will produce a delayed result, its use may improve interpretation of the most recent data point(s), and therefore reduce false positive and false negative messages.

The biometric device (100) measures at least one physiological parameter, and an abnormal condition is determined or predicted based on the analysis algorithm. The basic methodology employed by a processing module (108) is to ascertain the dynamics of the underlying homeostatic system involving at least one physiological parameter. The physiological parameter may be cardiovascular, metabolic, thermal, electrolyte balance, musculoskeletal, neurological, digestive, renal, etc.

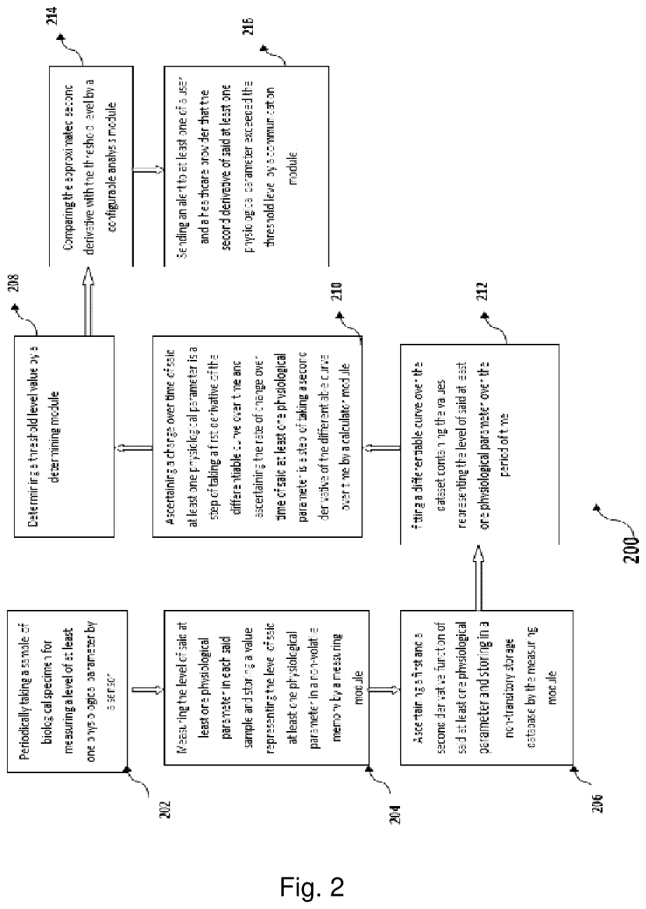

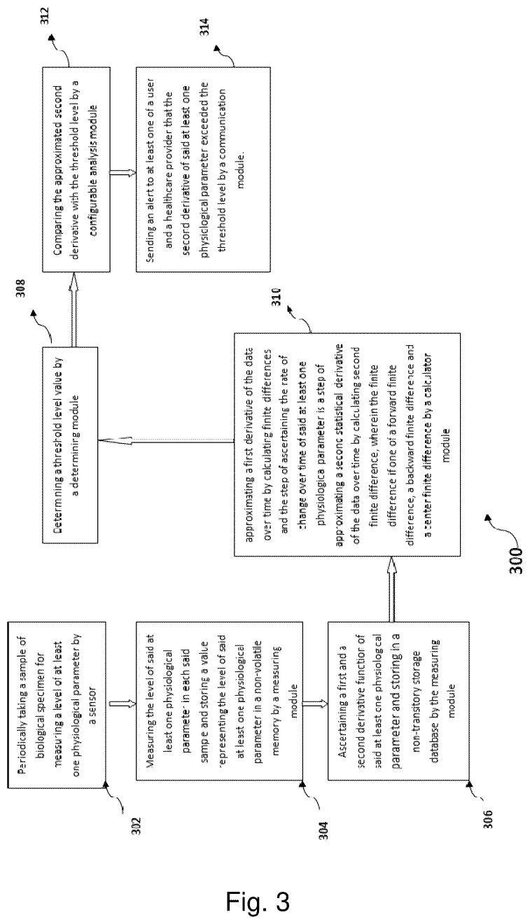

FIG. 2 and FIG. 3 illustrate flowcharts for a process for diagnosing and generating an alert in case of an abnormal condition of a user's health. FIG. 2 discloses a series of steps (200) of measuring physiological parameters using a biometric device (100). The steps include periodically taking a sample of biological specimen for measuring a level of at least one physiological parameter by a sensor at (202), measuring the level of said at least one physiological parameter in each sample and storing a value representing the level of said at least one physiological parameter in a non-volatile memory by a measuring module at (204), ascertaining a higher order dynamic response function of said at least one physiological parameter, determining if the higher order dynamic response function is actionable with respect to a medical or physiological criterion, and generating a message in dependence on the determining and storing data representing the physiological parameter in a memory by the measuring module at (206), fitting a differentiable curve over the dataset containing the values representing the level of said at least one physiological parameter over the period of time (212), ascertaining a change over time of said at least one physiological parameter is a step of taking a first derivative of the differentiable curve over time and ascertaining the rate of change over time of said at least one physiological parameter is a step of taking a second derivative of the differentiable curve over time by a calculator module (210), determining a threshold level value by a determining module (208), comparing the approximated second derivative with the threshold level by a configurable analysis module (214), sending an alert to at least one of a user and a healthcare provider that the second derivative of said at least one physiological parameter exceeded the threshold level by a communication module (216). As discussed above, rather than analyzing the second derivative per se, an equation or model of at least second-order may be analyzed, with actionable conditions determined, not based on a fixed threshold, but rather on a deviation from the model prediction.

FIG. 3 discloses a series of steps (300) for measuring physiological parameters using a biometric device with the application of a different mathematical approach (100). The steps include periodically taking a sample of biological specimen for measuring a level of at least one physiological parameter by a sensor at (302), measuring the level of said at least one physiological parameter in each sample and storing a value representing the level of said at least one physiological parameter in a non-volatile memory by a measuring module at (304), ascertaining a first and a second derivative function of said at least one physiological parameter and storing in a non-transitory storage database by the measuring module at (306), approximating a first derivative of the data over time by calculating finite differences and the step of ascertaining the rate of change over time of said at least one physiological parameter is a step of approximating a second statistical derivative of the data over time by calculating second finite difference, wherein the finite difference if one of a forward finite difference, a backward finite difference and a center finite difference by a calculator module (310), determining a threshold level value by a determining module (308), comparing the first and second derivative function with a reference function a configurable analysis module (312), sending an alert to at least one of a user and a healthcare provider that the first and second derivative of said at least one physiological parameter deviates from the reference function by a communication module (314).

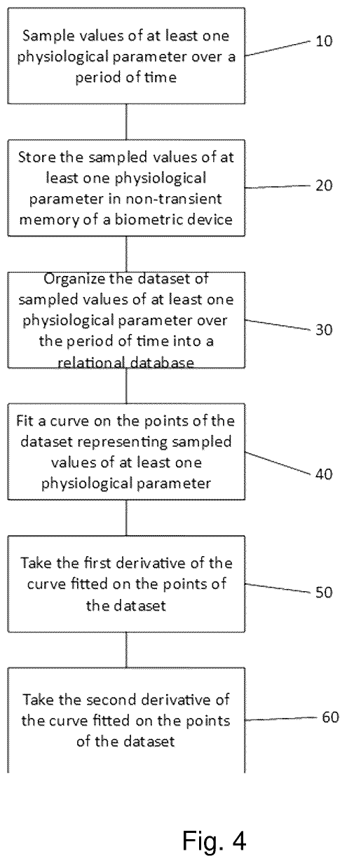

FIG. 4 shows a flowchart of a process, wherein sample values of at least one physiological parameter over a period of time 10. The sampled values of at least one physiological parameter are stored in non-transient memory of a biometric device 20. The dataset of sampled values of at least one physiological parameter over the period of time is organized into a relational database 30. A curve is fit on the points of the dataset representing sampled values of at least one physiological parameter 40. The first derivative 50 and the second derivative 60 are taken of the curve fitted on the points of the dataset.

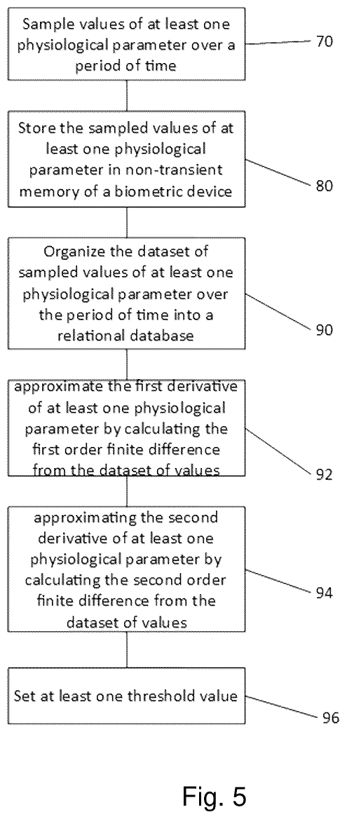

FIG. 5 shows a flowchart of a method in which values of at least one physiological parameter over a period of time are sampled 70. The sampled values of said at least one physiological parameter are stored in non-transient memory of a biometric device 80. The dataset of sampled values of at least one physiological parameter over the period of time is organized into a relational database 90. The first derivative of said at least one physiological parameter is approximated by calculating the first-order finite difference from the dataset of values 92. The second derivative of said at least one physiological parameter is approximated by calculating the second-order finite difference from the dataset of values 94. At least one threshold value is set 96.

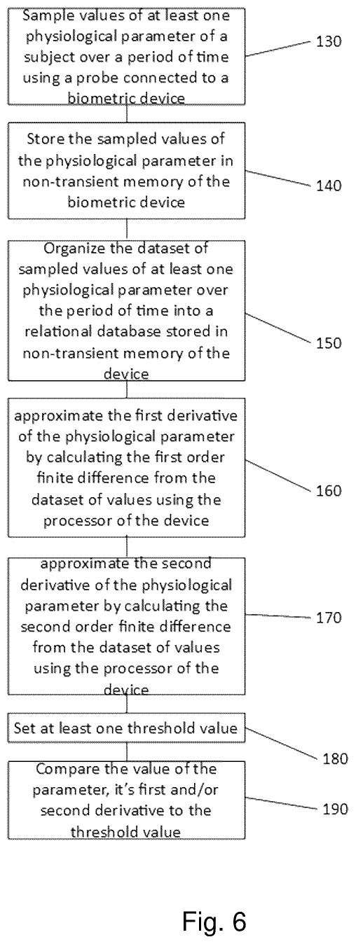

FIG. 6 shows a flowchart of a method in which values of at least one physiological parameter of a subject are sampled over a period of time using a probe connected to a biometric device 130. The sampled values of the physiological parameter are stored in non-transient memory of the biometric device 140. The dataset of sampled values of said at least one physiological parameter over the period of time is organized into a relational database stored in non-transient memory of the device 150. The first derivative of the physiological parameter is approximated by calculating the first-order finite difference from the dataset of values using the processor of the device 160. The second derivative of the physiological parameter is approximated by calculating the second-order finite difference from the dataset of values using the processor of the device 170. At least one threshold value is set 180. The value of the parameter, it's first and/or second derivative is compared to the threshold value 190.

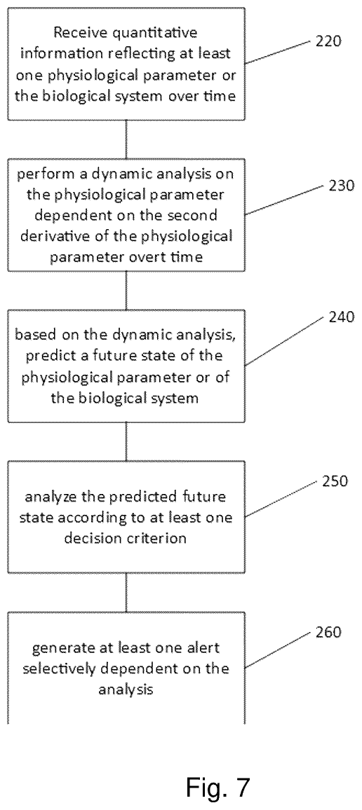

FIG. 7 shows a flowchart of a further method, in which quantitative information reflecting at least one physiological parameter or the biological system is received over time 220. A dynamic analysis is performed on the physiological parameter dependent on the second derivative of the physiological parameter over time 230. Based on the dynamic analysis, a future state of the physiological parameter or the biological system is predicted 240. The predicted future state is analyzed according to at least one decision criterion 250. At least one alert may be selectively generated dependent on the analysis 260.

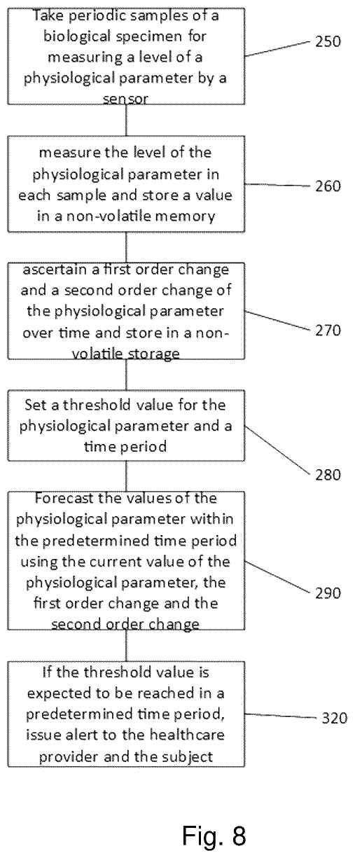

FIG. 8 shows a flowchart of a still further method, in which periodic samples of a biological specimen are taken for measuring a level of a physiological parameter by a sensor 250. The level of the physiological parameter in each sample is measured, and a value stored in a non-volatile memory 260. A first-order change (i.e., “velocity”) and a second-order change (i.e., “acceleration”) of the physiological parameter is ascertained over time and stored in a non-volatile storage 270. A threshold value is set for the physiological parameter and a time period 280. The values of the physiological parameter within the predetermined time period are forecasted using the current value of the physiological parameter, the first-order change and the second-order change 290. If the threshold value is expected to be reached in a predetermined time period, an alert is issued to the healthcare provider and the subject 320.

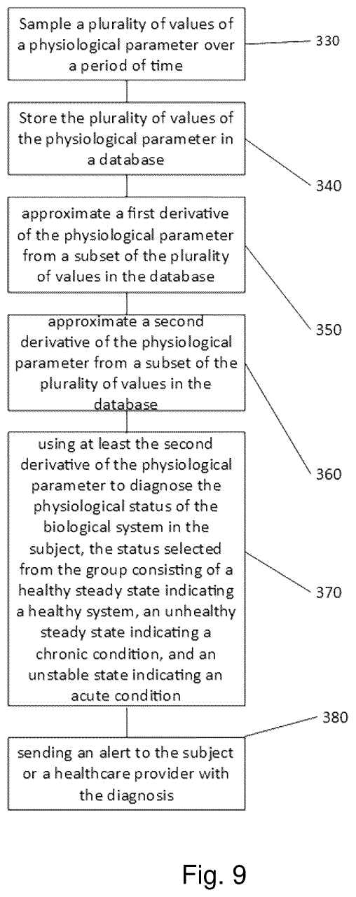

FIG. 9 shows another flowchart of a method, in which a plurality of values of a physiological parameter is sampled over a period of time 330. The plurality of values of the physiological parameter is stored in a database 340. A first derivative of the physiological parameter is approximated from a subset of the plurality of values in the database 350. A second derivative of the physiological parameter is approximated from a subset of the plurality of values in the database 360. At least the second derivative of the physiological parameter is used to diagnose the physiological status of the biological system in the subject, the status being selected from the group consisting of a healthy steady-state indicating a healthy system, an unhealthy steady-state indicating a chronic condition, and an unstable state indicating an acute condition 370. An alert is sent to the subject or a healthcare provider with the diagnosis 380.

As mentioned, there remains the foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments.

It is to be understood, however, that even though numerous characteristics and advantages of the present invention have been set forth in the foregoing description, together with details of the structure and function of the invention, the disclosure is illustrative only. Changes may be made in the details, especially in matters of shape, size, and arrangement of parts, a specific mathematical or statistical algorithm within the principles of the invention to the full extent indicated by the broad general meaning of the terms in which the appended claims are expressed.

What is claimed is:

1. An apparatus for analyzing a biological system of an individual, having a cyclic variation over time, comprising:
an input port configured to receive a series of measurements of the biological system over time;
a computational predictive statistical model, comprising at least a second order derivative term, configured to predict a future deviation from a normal state of the biological system of the individual, having the cyclic variation over time, based on the series of measurements of the biological system over time;
a processor configured to:
use the computational predictive statistical model to predict the future deviation, based on the series of measurements of the biological system over time;
determine a predicted significant deviation of the individual from the normal state with respect to a threshold for the individual; and
an output port configured to communicate a signal responsive to the predicted significant deviation of the individual from the normal state.

2. The apparatus according to claim 1, wherein signal responsive to the predicted significant deviation of the individual from the normal state comprises an alarm signal, further comprising an alarm configured to provide an indication of an alarm condition as an audible or electromagnetic emission.

3. The apparatus according to claim 1,
wherein the computational predictive statistical model is further configured to determine a statistical uncertainty of the prediction of the future deviation, and
the processor is further configured to determine the abnormal condition of the individual with respect to a joint function threshold for the individual, as a joint function of the predicted future deviation and the statistical uncertainty of the prediction of the future deviation.

4. The apparatus according to claim 1, wherein the biological system is a human heart and the cyclic variation over time comprises a cardiac cycle.

5. The apparatus according to claim 1, wherein the cyclic variation over time comprises human gait.

6. The apparatus according to claim 1, wherein the apparatus is wearable.

7. The apparatus according to claim 1, further comprising a sensor selected from the group consisting of at least one of a motion sensor, a pressure sensor, a photoplethysmography sensor, an electroencephalogram sensor, an electrocardiogram sensor, and an electromyogram sensor, wherein the input port is configured to receive the series of measurements from the sensor.

8. The apparatus according to claim 1, further comprising at least one actuator responsive to the analysis of the predicted future state, configured to correct the predicted significant deviation of the individual from the normal state.

9. The apparatus according to claim 1, wherein the computational predictive statistical model comprises an artificial neural network.

10. A method for analyzing a biological system of an individual, having a cyclic variation over time, comprising:
receiving a series of measurements of the biological system over time;
predicting a future deviation from a normal state of the biological system of the individual, having the cyclic variation over time, based on the series of measurements of the biological system over time, using a computational predictive statistical model, comprising at least a second order derivative term;
determining a predicted significant deviation of the individual from the normal state based on a relation of the predicted future deviation with an individual-specific threshold; and
communicating a signal responsive to the predicted significant deviation of the individual from the normal state.

11. The method according to claim 10, wherein signal responsive to the predicted significant deviation of the individual from the normal state comprises an alarm signal, further comprising indicating the alarm signal as an audible or electromagnetic emission.

12. The method according to claim 10,
wherein the computational predictive statistical model determines a statistical uncertainty of the prediction of the future deviation,
the method further comprising determining the abnormal condition of the individual based on a joint function of the predicted future deviation and the statistical uncertainty of the prediction of the future deviation and a joint function threshold for the individual.

13. The method according to claim 12, further comprising adaptively updating the computational predictive statistical model based on at least the series of measurements of the biological system over time.

14. The method according to claim 12, further comprising adaptively updating the joint function threshold for the individual based on at least the series of measurements of the biological system over time.

15. The method according to claim 10, wherein the cyclic variation over time comprises a cardiac cycle.

16. The method according to claim 10, wherein the cyclic variation over time comprises human gait.

17. The method according to claim 10, further comprising receiving the series of measurements of the biological system over time from at least one of a motion sensor, a pressure sensor, a photoplethysmography sensor, an electroencephalogram sensor, an electrocardiogram sensor, and an electromyogram sensor.

18. The method according to claim 10, further comprising automatically providing a treatment to the individual with at least one actuator, responsive to the signal.

19. The method according to claim 10, wherein the computational predictive statistical model comprises an artificial neural network, further comprising training the artificial neural network to distinguish between a predicted normal state of the individual and a predicted significant deviation of the normal state of the individual.

20. A method for monitoring a health of an individual, comprising:
automatically measuring at least one physiological parameter of a biological system of the individual having a cyclic variation over time, to produce a sequence of measurements over time;
storing the sequence of measurements over time in a memory;
analyzing the stored the sequence of measurements over time using a computational predictive statistical model, comprising at least a second order derivative term, to predict a future deviation from a normal state of the biological system of the individual having the cyclic variation over time;
determining a statistical reliability of the predicted future deviation from a normal state;
determining an action dependent on a joint function of the predicted future deviation from a normal state and the determined statistical reliability of the predicted future deviation from a normal state, the action being at least one of an alert and a correction of the predicted future deviation from a normal state; and
at least one of communicating the alert and automatically correcting the predicted future deviation from a normal state.
